(19) United States
(12) Patent Application Publication
(10) Pub. No.: US 2024/0191053 A1
(43) Pub. Date: Jun. 13, 2024

(54) SYSTEMS, MICROORGANISMS, OR METHODS FOR WASTE PET VALORIZATION

(71) Applicant: WASHINGTON UNIVERSITY, St. Louis, MO (US)

(72) Inventors: Tae Seok Moon, St. Louis, MO (US); Jinjin Diao, St. Louis, MO (US); Yifeng Hu, St. Louis, MO (US); Kirsten Davis, St. Louis, MO (US)

(73) Assignee: Washington University, St. Louis, MO (US)

(21) Appl. No.: 18/555,230

(22) PCT Filed: Apr. 12, 2022
(86) PCT No.: PCT/US22/24500

§ 371 Date: Oct. 12, 2023

Related U.S. Application Data
(60) Provisional application No. 63/322,365, filed on Mar. 22, 2022.
(60) Provisional application No. 63/173,635, filed on Apr. 12, 2021.

(51) Int. Cl. C08J 11/10; C12N 9/10; C12N 9/88; C12N 9/90; C12N 15/52

(52) U.S. Cl. CPC C08J 11/105; C12N 9/1022; C12N 9/88; C12N 9/90; C12N 15/52; C12Y 202/01007; C12Y 401/01063; C12Y 503/03002

(57) ABSTRACT

Among the various aspects of the present disclosure is the provision of methods, systems, and Rhodococcus sp. strains for the upcycling of polyethylene terephthalate) (PET). An aspect of the present disclosure provides for a system for waste PET valorization comprising: a microorganism capable of growing on PET hydrolysis products, such as PET hydrolysate. PET products from chemical hydrolysis, or alkaline hydrolysis products of PET as a carbon source.

### CROSS-REFERENCE TO RELATED APPLICATIONS

This application claims priority from U.S. Provisional Application Ser. No. 63/173,635 filed on 12 Apr. 2021 and U.S. Provisional Application Ser. No. 63/322,365 filed on 22 Mar. 2022, which are incorporated herein by reference in their entireties.

### STATEMENT REGARDING FEDERALLY SPONSORED RESEARCH OR DEVELOPMENT

This invention was made with government support under DE-SC0018324 and DE-SC0022003 awarded by the Department of Energy. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name 019715-WO_Sequence_Listing_ST25.txt created on 6 Apr. 2022; 107,713 bytes). The subject matter of the Sequence Listing is incorporated herein by reference in its entirety.

### FIELD

The present disclosure generally relates to upcycling of poly(ethylene terephthalate) (PET).

### SUMMARY

Among the various aspects of the present disclosure is the provision of methods, systems, and microorganisms for the upcycling of poly(ethylene terephthalate) (PET). An aspect of the present disclosure provides for a system for waste poly(ethylene terephthalate) (PET) valorization comprising: a microorganism capable of growing on PET hydrolysis products, such as PET hydrolysate, PET products from chemical hydrolysis, or alkaline hydrolysis products of PET as a carbon source. Another aspect of the present disclosure provides for a microorganism for waste poly(ethylene terephthalate) (PET) valorization comprising: a Rhodococcus strain (e.g., RPET) capable of growing on PET hydrolysis products, such as PET hydrolysate or alkaline hydrolysis products of PET as a carbon source. Yet another aspect of the present disclosure provides for a method of waste poly(ethylene terephthalate) (PET) valorization comprising: providing a microorganism capable of growing on PET hydrolysis products, such as PET hydrolysate or alkaline hydrolysis products of PET as a carbon source. In some embodiments, PET is depolymerized into PET hydrolysis products, such as PET hydrolysate or alkaline hydrolysis products of PET or a carbon source (e.g., terephthalic acid (TPA) and ethylene glycol (EG)). An aspect of the present disclosure provides for a system for waste polyethylene terephthalate (PET) valorization. In some embodiments, the system comprises a wild type Rhodococcus sp. strain or engineered Rhodococcus sp. strain capable of growing in media comprising an PET hydrolysis products, such as PET hydrolysate or alkaline hydrolysis products of PET as a carbon source. In some embodiments, the system comprises media comprising an PET hydrolysis products, such as PET hydrolysate or alkaline hydrolysis products of PET, wherein the hydrolysis product of PET comprises terephthalic acid (TPA) or ethylene glycol (EG) or both. In some embodiments, if the system comprises an engineered the engineered Rhodococcus sp. strain, the engineered Rhodococcus sp. strain is engineered to introduce a TPA or EG degradation pathway or engineered to include product formation pathways via an artificial DNA construct (TPA pathway(s) introduced can be native or non-native). Another aspect of the present disclosure provides for an engineered Rhodococcus sp. strain transformed with an artificial DNA construct comprising, as operably associated components in the 5′ to 3′ direction of transcription: (a) a promoter functional in the Rhodococcus sp. strain; or (b) one or more polynucleotides selected from: (i) a first polynucleotide comprising a nucleotide sequence encoding a first polypeptide having prenyltransferase—(CrtE), phytoene synthase—(CrtB), and/or phytoene desaturase—(CrtI) enzymatic activity, optionally together as CrtEBI or as separate polynucleotide(s); (ii) a second polynucleotide comprising a nucleotide sequence encoding a second polypeptide having 1-deoxyxylulose-5-phosphate synthase enzymatic activity; (iii) a third polynucleotide comprising a nucleotide sequence encoding a third polypeptide having isopentenyl pyrophosphate isomerase enzymatic activity; and/or (iv) a fourth polynucleotide comprising a nucleotide sequence encoding a fourth polypeptide having 1-deoxyxylulose-5-phosphate synthase and isopentenyl pyrophosphate isomerase enzymatic activity; (c) a transcriptional termination sequence. In some embodiments, the crtL-b gene in the wild type Rhodococcus sp. strain is knocked out or knocked down or lycopene beta cyclase (CrtL-b) is downregulated or underexpressed. In some embodiments, the engineering of the Rhodococcus sp. strain results in an increase in dimethylallyl diphosphate (DMAPP) and isopentenyl pyrophosphate (IPP) and an increase in accumulation of lycopene. Yet another aspect of the present disclosure provides for an artificial DNA construct comprising, as operably associated components in the 5′ to 3′ direction of transcription: (a) a promoter functional in a Rhodococcus sp. strain; or (b) one or more polynucleotides comprising a nucleotide sequence encoding a polypeptide having one or more enzymatic activities or an enzyme. In some embodiments, the enzyme or enzymatic activity is selected from one or more of 2-pyrone synthase; 6-ethylsalycilic acid synthase; acetyl-CoA carboxylase; aconitate hydratase; CAT 1,2-dioxygenase activity; CAT 2,3-dioxygenase activity; citrate synthase; DOXP synthase (Dxs); fatty acid synthase; fumarate hydratase; glyceraldehyde-3-phosphate (G3P) dehydrogenase; geranyl diphosphate synthase; HMB-PP reductase; isocitrate dehydrogenase; isocitrate lyase; isopentenyl pyrophosphate isomerase; lycopene β cyclase; malate dehydrogenase; malate synthase; malic enzyme; muconate cycloisomerase activity; O-methyl transferase activity; oxoglutarate dehydrogenase; PCA 3,4-dioxygenase activity; PCA decarboxylase; PEP carboxykinase; PEP carboxylase; phenol hydroxylase; phosphoglycerate kinase; phosphoglycerate mutase; phosphopyruvate hydratase; p-hydroxybenzoate hydroxylase; pyruvate dehydrogenase; pyruvate kinase; succinate dehydrogenase; succinyl CoA synthetase; terephthalic acid (TPA) dioxygenase or combinations thereof. In some embodiments, the enzyme or enzymatic activity is selected from one or more of geranylgeranyl diphosphate synthase (CrtE); phytoene synthase (CrtB); phytoene desaturase (CrtI); lycopene beta-cyclase (CrtL-b); or combinations thereof; 1-deoxyxylulose-5-phosphate synthase (dxs); 1-deoxy-d-xylulose 5-phosphate reductoisomerase (dxr); 4-diphosphocytidyl-2C-methyl-D-erythritol synthase (ispD); isopentenyl monophosphate kinase (ispE); 2-C-methyl-D-erythritol 2,4-cyclodiphosphate synthase (ispF); 4-hydroxy-3-methylbut-2-en-1-yl diphosphate synthase (ispG); 4-hydroxy-3-methylbut-2-enyl diphosphate reductase (ispH); isopentenyl pyrophosphate isomerase (idi); or combinations thereof. In yet another aspect, the present disclosure provides for an engineered Rhodococcus sp. strain transformed with the construct of any one of the preceding embodiments or aspects or a Rhodococcus sp. strain engineered to introduce a TPA or EG degradation pathway or engineered to include product formation pathways via an artificial DNA construct, and accumulates increased levels of a target product (e.g., a target product, a target product derivative, or a target product precursor) produced from a PET hydrosylate carbon source compared to the Rhodococcus sp. strain not comprising the artificial DNA construct. In some embodiments, the target product or target product precursors are selected from one or more of 1-hydroxy-2-methyl-2-butenyl-4-diphosphate; 1-deoxy-D-xylulose-5-phosphate; 2-oxoglutarate; acetyl-CoA; catechol (CAT); citrate; dimethylallyl pyrophosphate; fatty acids; fumarate; gallic acid (GA); geranyl diphosphate; glyceraldehyde-3-phosphate (G3P); glycerate-1,3-diphosphate; glycerate-2-phosphate; glycerate-3-phosphate; glyoxylate; isocitrate; isopentyl pyrophosphate; lycopene; malate; malonyl-CoA; muconate or muconic acid (MA); oxaloacetate; phosphophenylpyruvate (PEP); protocatechuate or protocatechuic acid (PCA); pyrogallol; pyruvate; succinate; succinyl-CoA; triacetic acid lactone (TAL); vanillic acid (VA); β-carotene; or γ-carotene; or combinations thereof. In some embodiments, the target product or target product precursors are selected from one or more of 4-carboxy-2-hydroxymuconate semialdehyde (4CHMS); 5-carboxy-2-hydroxymuconate-6-semialdehyde (5CHMS); or 3-carboxy-cis,cis-muconate (CM); or combinations thereof. In some embodiments, the target product or target product precursors are selected from one or more of 2-phosphoglycerate; acetyl-CoA; citrate; farnesyl pyrophosphate (FPP); fumarate; geranyl pyrophosphate (GPP); glyceraldehyde-3-phosphate (G3P); glyoxylate; isocitrate; lycopene; malate; oxaloacetate; pyruvate (PYR); succinate; succinyl-CoA; α-ketogluterate; β-carotene; or combinations thereof. In some embodiments, the target product or target product precursors are selected from one or more of 1-deoxy-D-xylulose-5-phosphate (DXP); 1-hydroxy-2-methyl-2-(E)-butenyl-4-diphosphate (HMB-PP); 2-C-methyl-D-erythritol 4-phosphate (MEP); 2-C-methyl-D-erythritol-2,4-cyclo-diphosphate (MEC); 4-diphosphocytidyl-2-C-methyl-D-erythritol (CDP-ME); 4-diphosphocytidyl-2-C-methyl-D-erythritol 2-phosphate (CDP-MEP); dimethylallyl diphosphate (DMAPP); isopentenyl pyrophosphate (IPP); or combinations thereof. In some embodiments, the target product or target product precursors are selected from one or more of protocatechuic acid (PCA), gallic acid (GA), pyrogallol, catechol, muconic acid (MA) (e.g., cis, cis-MA, cis, trans-MA and trans, trans-MA), or vanillic acid (VA). In yet another aspect, the present disclosure provides for an engineered Rhodococcus sp. strain, wherein the engineered Rhodococcus sp. strain expresses or overexpresses genes encoding an enzyme. In some embodiments, the enzyme is selected from one or more of 1-deoxy-d-xylulose 5-phosphate reductoisomerase (dxr); 4-diphosphocytidyl-2C-methyl-D-erythritol synthase (ispD); isopentenyl monophosphate kinase (ispE); 2-C-methyl-D-erythritol 2,4-cyclodiphosphate synthase (ispF); 4-hydroxy-3-methylbut-2-en-1-yl diphosphate synthase (ispG); pyrophosphate synthase; geranylgeranyl diphosphate synthase; phytoene synthase; phytoene desaturase; zeta-carotene isomerase; zeta-carotene desaturase; carotenoid isomerase; 2-pyrone synthase; 6-ethylsalycilic acid synthase; acetyl-CoA carboxylase; aconitate hydratase; CAT 1,2-dioxygenase activity; CAT 2,3-dioxygenase activity; citrate synthase; DOXP synthase (Dxs); fatty acid synthase; fumarate hydratase; glyceraldehyde-3-phosphate (G3P) dehydrogenase; geranyl diphosphate synthase; HMB-PP reductase; isocitrate dehydrogenase; isocitrate lyase; isopentenyl pyrophosphate isomerase; lycopene β cyclase; malate dehydrogenase; malate synthase; malic enzyme; muconate cycloisomerase activity; O-methyl transferase activity; oxoglutarate dehydrogenase; PCA 3,4-dioxygenase activity; PCA decarboxylase; PEP carboxykinase; PEP carboxylase; phenol hydroxylase; phosphoglycerate kinase; phosphoglycerate mutase; phosphopyruvate hydratase; p-hydroxybenzoate hydroxylase; pyruvate dehydrogenase; pyruvate kinase; succinate dehydrogenase; succinyl CoA synthetase; terephthalic acid (TPA) dioxygenase or combinations thereof. In some embodiments, the enzyme is selected from one or more of geranylgeranyl diphosphate synthase (CrtE); phytoene synthase (CrtB); phytoene desaturase (CrtI); lycopene beta-cyclase (CrtL-b); or combinations thereof. In some embodiments, the enzyme is selected from one or more of 1-deoxyxylulose-5-phosphate synthase (dxs); 1-deoxy-d-xylulose 5-phosphate reductoisomerase (dxr); 4-diphosphocytidyl-2C-methyl-D-erythritol synthase (ispD); isopentenyl monophosphate kinase (ispE); 2-C-methyl-D-erythritol 2,4-cyclodiphosphate synthase (ispF); 4-hydroxy-3-methylbut-2-en-1-yl diphosphate synthase (ispG); 4-hydroxy-3-methylbut-2-enyl diphosphate reductase (ispH); isopentenyl pyrophosphate isomerase (idi); or combinations thereof. In some embodiments, the engineered Rhodococcus sp. strain accumulates increased levels of a target product when grown on PET hydrosylate compared to a wild type Rhodococcus sp. strain. In yet another aspect, the present disclosure provides for an engineered Rhodococcus sp. strain of any one of the preceding aspects or embodiments wherein the genes transcribing the following enzymes are knocked out, knocked down, downregulated, or underexpressed compared to the wild type Rhodococcus sp. strain. In some embodiments, the enzyme is selected from one or more of 1-deoxy-d-xylulose 5-phosphate reductoisomerase (dxr); 4-diphosphocytidyl-2C-methyl-D-erythritol synthase (ispD); isopentenyl monophosphate kinase (ispE); 2-C-methyl-D-erythritol 2,4-cyclodiphosphate synthase (ispF); 4-hydroxy-3-methylbut-2-en-1-yl diphosphate synthase (ispG); pyrophosphate synthase; geranylgeranyl diphosphate synthase; phytoene synthase; phytoene desaturase; zeta-carotene isomerase; zeta-carotene desaturase; carotenoid isomerase; 2-pyrone synthase; 6-ethylsalycilic acid synthase; acetyl-CoA carboxylase; aconitate hydratase; CAT 1,2-dioxygenase activity; CAT 2,3-dioxygenase activity; citrate synthase; DOXP synthase (Dxs); fatty acid synthase; fumarate hydratase; glyceraldehyde-3-phosphate (G3P) dehydrogenase; geranyl diphosphate synthase; HMB-PP reductase; isocitrate dehydrogenase; isocitrate lyase; isopentenyl pyrophosphate isomerase; lycopene β cyclase; malate dehydrogenase; malate synthase; malic enzyme; muconate cycloisomerase activity; O-methyl transferase activity; oxoglutarate dehydrogenase; PCA 3,4-dioxygenase activity; PCA decarboxylase; PEP carboxykinase; PEP carboxylase; phenol hydroxylase; phosphoglycerate kinase; phosphoglycerate mutase; phosphopyruvate hydratase; p-hydroxybenzoate hydroxylase; pyruvate dehydrogenase; pyruvate kinase; succinate dehydrogenase; succinyl CoA synthetase; terephthalic acid (TPA) dioxygenase or combinations thereof. In some embodiments, the enzyme is selected from one or more of geranylgeranyl diphosphate synthase (CrtE); phytoene synthase (CrtB); phytoene desaturase (CrtI); lycopene beta-cyclase (CrtL-b); or combinations thereof; 1-deoxyxylulose-5-phosphate synthase (dxs); 1-deoxy-d-xylulose 5-phosphate reductoisomerase (dxr); 4-diphosphocytidyl-2C-methyl-D-erythritol synthase (ispD); isopentenyl monophosphate kinase (ispE); 2-C-methyl-D-erythritol 2,4-cyclodiphosphate synthase (ispF); 4-hydroxy-3-methylbut-2-en-1-yl diphosphate synthase (ispG); 4-hydroxy-3-methylbut-2-enyl diphosphate reductase (ispH); isopentenyl pyrophosphate isomerase (idi); or combinations thereof. In some embodiments, the engineered Rhodococcus sp. strain is engineered to produce a target product not produced in a wild type Rhodococcus sp. strain or engineered to overproduce the target product produced in a wild type Rhodococcus sp. strain compared to wild type Rhodococcus sp. strain. In some embodiments, the engineered Rhodococcus sp. strain is engineered to produce a target product not produced in a wild type Rhodococcus sp. strain or engineered to overproduce the target product produced in a wild type Rhodococcus sp. strain compared to wild type Rhodococcus sp. strain, wherein the target product is selected from or derived from carotenoids, lycopene, muconate, or β-carotene. In some embodiments, genes or enzymes are identified in a pathway for a target product (optionally, a carotenoid or a muconate), are knocked out, knocked down, expressed, overexpressed, downregulated, or upregulated to increase accumulation or production of the target product or precursor compared to the accumulation of the target product or precursor in a wild type Rhodococcus sp. strain. In some embodiments, the artificial DNA construct is a self-replicating primer. In some embodiments, the artificial DNA construct is capable of expressing native or non-native genes encoding enzymatically active polypeptides. In some embodiments, crtL-b is knocked out. In some embodiments, a C. glutamicum-sourced dxs gene is over expressed. In some embodiments, a C. glutamicum isopentenyl pyrophosphate isomerase gene (idi) is overexpressed. In some embodiments, idi and dxs is co-expressed. In some embodiments, CrtEBI is expressed. In some embodiments, IPP and DMAPP precursor to lycopene production is optimized. In some embodiments, the engineered Rhodococcus sp. strain is engineered to produce a target product not produced in a wild type Rhodococcus sp. strain or engineered to overproduce the target product produced in a wild type Rhodococcus sp. strain compared to wild type Rhodococcus sp. strain. In some embodiments, the target product is lycopene and the lycopene accumulation is greater than in wild type Rhodococcus sp. strain; the target product is β-carotene and the β-carotene accumulation is greater than in wild type Rhodococcus sp. strain. In some embodiments, the target product is muconate and the muconate accumulation is greater than in wild type Rhodococcus sp. strain. In yet another aspect, the present disclosure provides for a method for waste poly(ethylene terephthalate) (PET) valorization or generating a target product comprising: providing a wild type or engineered Rhodococcus sp. strain capable of growing on PET hydrolysis products, such as PET hydrolysate or alkaline hydrolysis products of PET as a carbon source; or incubating the wild type or engineered Rhodococcus sp. strain in media comprising an PET hydrolysis products, such as PET hydrolysate or alkaline hydrolysis products of PET, wherein the hydrolysis product of PET comprises terephthalic acid (TPA) or ethylene glycol (EG) or both. In some embodiments, the wild type or engineered Rhodococcus sp. strain is incubated for an amount of time sufficient to accumulate a target product. In some embodiments, the media is neutralized and the PH neutralized media supports growth of the Rhodococcus sp. strain, optionally without any purification or sterilization step except for dilution to make monomer mixtures with necessary salts. In some embodiments, aqueous NaOH without any additional catalyst, resulting in up to about 90% depolymerization at between about 90° C. and 180° C. for between about 1.5 and 2 hours. In some embodiments, the amount of time sufficient to accumulate a target product is overnight, 2 hours, 4 hours, 12 hours, two days, three days, or more, or until colonies are visible or sufficient cells grow in a culture, optionally 2D, 3D, or liquid culture, for example. In some embodiments, the wild type or engineered Rhodococcus sp. strain is incubated at a temperature sufficient to culture the Rhodococcus sp. strain. In some embodiments, the temperature sufficient to culture cells is 30° C. In some embodiments, the wild type or engineered Rhodococcus sp. strain is wild type or engineered Rhodococcus sp. strain of any one of the preceding aspects or embodiments. In some embodiments, the artificial DNA construct of any one of the preceding aspects or embodiments and the Rhodococcus sp. strain of any one of the preceding aspects or embodiments, wherein the engineered Rhodococcus sp. strain is engineered to produce a target product not produced in a wild type Rhodococcus sp. strain or engineered to overproduce the target product produced in a wild type Rhodococcus sp. strain compared to wild type Rhodococcus sp. strain. In some embodiments, the target product is accumulated at a 500-fold increase or more compared to the wild type Rhodococcus sp. strain. In some embodiments, the target product is accumulated at an amount of 1200 μg/L or more lycopene from PET hydrolysate alone. In some embodiments, the method further comprises producing PET hydrolysate carbon source comprising depolymerizing PET via alkaline hydrolysis into monomers, wherein the monomers comprise TPA and EG; wherein the PET hydrolysate is used as a carbon source to support cell growth of a Rhodococcus sp. strain. In some embodiments, the method further comprises providing exogenous arabinose as an inducer or glucose as an additional carbon source. In some embodiments, the engineered Rhodococcus sp. strain of any one of the preceding aspects or embodiments, or the method of the preceding aspects or embodiments, wherein the engineered Rhodococcus sp. strain is a transgenic Rhodococcus sp. strain. In some embodiments, the PET hydrolysis product is an alkaline hydrolysis product, generated by alkaline hydrolysis of PET and the PET hydrolysate or the alkaline hydrolysis product comprises TPA and EG. In some embodiments, the wild type or engineered Rhodococcus sp. strain is capable of increased synthesis of a product compared to Pseudomonas umsongensis GO16, Escherichia coli, Pseudomonas putida KT2440, Corynebacterium glutamicum, Pseudomonas putida KT2440, and Rhodococcus opacus PD630. In some embodiments, the wild type or engineered Rhodococcus sp. strain is tolerant to osmotic stress and has the ability to utilize PET hydrolysate comprising carbon sources, TPA and EG. In some embodiments, the media comprises about equimolar TPA and EG. In some embodiments, the media comprises about equimolar TPA and EG at a concentration of: between about 5 mM and 400 mM each, between about 40 mM to 100 mM each, between about 40 mM to 300 mM each, or between about 150 to 300 mM each. In some embodiments, the media comprises about equimolar TPA and EG at a concentration of: 15 mM each; 20 mM each; up to about 300 mM; or up to about 400 mM each. In some embodiments, the media comprises Na⁺ or K⁺ and the Na⁺ or K⁺ concentration is between about 80 mM to 200 mM or no more than 200 mM Na⁺ or K⁺. In some embodiments, the PET hydrolysis product is diluted to at least 5-fold, optionally, 10-fold or 20-fold. In some embodiments, PET is depolymerized into PET hydrolysis products, such as PET hydrolysate or alkaline hydrolysis products of PET or a carbon source comprising terephthalic acid (TPA) and ethylene glycol (EG). In some embodiments, TPA and EG are biologically converted into high-value products or a product of higher economic value than PET, such as carotenoids and muconate, aromatic or aromatic-derived chemicals, such as, protocatechuic acid (PCA), gallic acid (GA), pyrogallol, catechol, muconic acid (MA) (e.g., cis,cis-MA, cis, trans-MA and trans, trans-MA), and vanillic acid (VA). In some embodiments, the Rhodococcus sp. strain is Rhodococcus jostii capable of growing on the PET hydrolysis products, such as PET hydrolysate or alkaline hydrolysis products of PET as the carbon source without any purification step. In some embodiments, TPA and EG are at extremely high concentrations (e.g., up to 0.3 M each, total 0.6 M) and, optionally, high osmolarity resulting from alkaline hydrolysis and pH neutralization. In some embodiments, 2 mol NaOH per 1 mol released TPA is added to maintain pH ˜8, resulting in high osmolarity. In some embodiments, the Rhodococcus sp. strain yielded up to 37% biomass per used PET (e.g., 37 g DCW/g PET). In some embodiments, the Rhodococcus sp. strain comprises a heterologous PCA decarboxylase, optionally, and the heterologous PCA decarboxylases are optimized for efficient TPA-to-MA (muconate) production. In some embodiments, the method of any one of the preceding aspects or embodiments, wherein the Rhodococcus sp. strain is engineered by introducing a TPA or EG degradation pathway or engineered to include product formation pathways into the Rhodococcus sp. strain. In some embodiments, the Rhodococcus sp. strain is engineered to express or overexpress CrtEBI (CrtE, geranylgeranyl diphosphate synthase; CrtB, phytoene synthase; and CrtI, Phytoene desaturase) for the production or accumulation of lycopene. In some embodiments, the method of any one of the preceding aspects or embodiments, wherein the Rhodococcus sp. strain is engineered to knock out or knock down CrtL-b (lycopene beta-cyclase) expression. In yet another aspect, the present disclosure provides for a Rhodococcus sp. strain transformed with a polynucleotide encoding a polypeptide, the polynucleotide comprising one or more of an amino acid sequence selected from one or more of SEQ ID NO: 12, 13, 14, or 15 or a functional fragment thereof or a sequence 90% identical to SEQ ID NO: 12, 13, 14, or 15 or a functional fragment thereof; the polypeptide having DOXP synthase (dxs); isopentenyl pyrophosphate isomerase (idi); geranylgeranyl diphosphate synthase (CrtE); phytoene synthase (CrtB); phytoene desaturase (CrtI); or crtEBI activity; and optionally CrtL-b knocked out (ΔcrtL-b) or CrtL-b knocked down. wherein the Rhodococcus sp. strain produces lycopene from PET hydrolysate.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1. Open-loop upcycling of PET to value-added chemicals by an engineered RPET strain. a, A conceptual overview of the open-loop upcycling of PET to bulk/fine chemicals. PET depolymerization is catalyzed by alkaline hydrolysis, and the resulting monomers TPA and EG are used as feedstocks for bioconversion to produce target chemicals. b, Alkaline hydrolysis of PET to generate its corresponding monomers TPA and EG. c, The catabolic pathways of TPA and EG for supporting the cell growth of RPET strain. TPA is converted to Acetyl-CoA and succinate to fuel the TCA cycle via the β-ketoadipate pathway. The dashed, grey line indicates that the EG degradation pathway in this strain is still uncharacterized. The central metabolites in RPET can be selected as the starting precursors for the biosynthesis of target chemicals through rational metabolic engineering strategies.

FIG. 2. Screening and testing bacterial strains for growth on TPA, EG, and PET hydrolysate. a, Several strains were screened for the ability to grow by using TPA or EG as sole carbon source. The growth curves (OD₆₀₀) of RPET in minimal medium plus 15 mM TPA or 15 mM EG as sole carbon source are depicted. b, Comparison of cell growth of the RPET strain in different concentrations of simulated hydrolysate, ranging from 40 mM each TPA and EG to 300 mM each. c, Impact of the PET hydrolysate generated from alkaline hydrolysis on the cell growth of RPET. 6 g PET powder was suspended in 60 mL 2 M NaOH solution, and then subjected to hydrolysis. The resulting hydrolysate was diluted by a factor of 5, 10 or 20, and then fed to the wild-type RPET strain as carbon source. All values represent the mean of triplicate cultures, with error bars depicting the standard deviation from that mean.

FIG. 3. Engineering the RPET strain for the conversion of TPA and EG to lycopene. a, The proposed central carbon metabolism in RPET when using TPA and EG as carbon sources; the boxes indicate the compounds produced by TPA (pink) and EG (dotted grey) catabolismb, Engineering the endogenous carotenoid biosynthetic pathway for the production of lycopene. Pink and the A symbol indicate a gene deletion; aqua text indicates gene overexpression. Abbreviations: CrtE, geranylgeranyl diphosphate synthase; CrtB, phytoene synthase; CrtI, Phytoene desaturase; G3P, glyceraldehyde-3-phosphate; PYR, pyruvate; MEP, 2-methylerythritol 4-phosphate pathway; GPP, geranyl pyrophosphate; FPP, farnesyl pyrophosphate; CrtL-b, lycopene beta-cyclase. c, HPLC analysis of lycopene production in RPET and its derived strains. All strains were cultivated in 250 mL flasks with 50 mL minimal medium plus 20 mM TPA and 20 mM EG as carbon sources. All values represent the mean of three biological replicates, with error bars depicting the standard deviation from that mean.

FIG. 4. Optimization of the IPP precursor supply for the production of lycopene. a, Schematic of the endogenous MEP pathway. Aqua text indicates a gene targeted for overexpression. Abbreviations: dxs, 1-deoxyxylulose-5-phosphate synthase; dxr, 1-deoxy-d-xylulose 5-phosphate reductoisomerase; ispD, 4-diphosphocytidyl-2C-methyl-D-erythritol synthase; ispE, isopentenyl monophosphate kinase; ispF, 2-C-methyl-D-erythritol 2,4-cyclodiphosphate synthase; ispG, 4-hydroxy-3-methylbut-2-en-1-yl diphosphate synthase; ispH, 4-hydroxy-3-methylbut-2-enyl diphosphate reductase; idi, isopentenyl pyrophosphate isomerase; DXP, 1-Deoxy-D-xylulose-5-phosphate; MEP, 2-C-methyl-D-erythritol 4-phosphate; CDP-ME, 4-diphosphocytidyl-2-C-methyl-D-erythritol; CDP-MEP, 4-diphosphocytidyl-2-C-methyl-D-erythritol 2-phosphate; MEC, 2-C-methyl-D-erythritol-2,4-cyclo-diphosphate; HMB-PP, 1-hydroxy-2-methyl-2-(E)-butenyl-4-diphosphate; DMAPP, dimethylallyl diphosphate; IPP, isopentenyl pyrophosphate. b, Schematic overview of the overexpression of the key genes in the MEP pathway. The resulting plasmids (pJD-095, pJD-096, and pJD-097) were transformed into Strain S3 to produce Strains S4-S6. c, Lycopene production in engineered RPET strains relative to overexpression of different genes. All strains were cultivated in the 250 mL flask with 50 mL minimal medium plus 20 mM TPA and 20 mM EG as carbon sources. For overexpression of the synthetic crtEBI operon (Strain S7 only), 50 mM arabinose was added as an inducer. All values represent the mean of three biological replicates, with error bars depicting the standard deviation from that mean.

FIG. 5. Bio-upcycling of PET to lycopene. a, The scheme of the bio-upcycling route for PET waste to lycopene. PET waste was depolymerized via alkaline hydrolysis, and then centrifuged to remove the unreacted residues; the clear supernatant that contains the resulting monomers was neutralized to pH=7.0, and the neutralized PET hydrolysate was used as carbon sources to feed the engineered RPET strain for the production of lycopene. b, Cell growth and depletion of TPA and EG substrates by Strain S6 with PET hydrolysate as sole carbon source. The engineered RPET Strain S6 was cultivated in 250 ml flask with 50 mL minimal medium at 30° C. The PET hydrolysate generated from alkaline hydrolysis was added to a concentration of approximately 43 mM each of TPA and EG (10-fold dilution; Na⁺ concentration was 200 mM). At each time point, a 200 μL sample of the culture volume was taken to measure the cell density (OD); the cell suspensions were then centrifuged, and concentrations of the TPA and EG in the supernatant were determined by HPLC. c, Lycopene production of the WT and Strain S6 using PET hydrolysate as sole carbon source. After fermentation, 5 mL cells were centrifuged and washed with deionized H₂O twice. The lyophilized cell pellet was weighed and then subjected to lycopene assay. All values represent the mean of triplicate cultures, with error bars depicting the standard deviation from that mean.

FIG. 6. Screening candidate chassis strains for growth using TPA or EG as sole carbon source. In addition to RPET, several strains were screened for growth in minimal medium plus 15 mM TPA (a) or 15 mM EG (b) as sole carbon source. All values represent the mean of triplicate cultures, with error bars depicting the standard deviation from that mean.

FIG. 7. Utilization profiles of the TPA and EG monomers in the PET hydrolysate by WT RPET strain. A glucose-grown WT cell culture was used as the inoculum for cultures grown in minimal medium with different concentrations of PET as carbon sources: a, 5-fold dilution of PET hydrolysate; b, 10-fold dilution of PET hydrolysate; c, 20-fold dilution of PET hydrolysate. At each time point, a 200 μL sample of the cell suspension was centrifuged to measure the concentrations of TPA and EG in the supernatant by HPLC. All values represent the mean of triplicate cultures, with error bars depicting the standard deviation from that mean.

FIG. 8. Genetic engineering of RPET for the production of lycopene from simulated PET hydrolysate. a, The proposed carotenoid biosynthetic pathway in RPET. b, HPLC counts versus retention time is shown for the detection of lycopene (lavender dashed line), β-carotene (orange dashed line), and the three unknown peaks are marked with black dashed lines. c, Lycopene production of the mutant Strain S3. All the strains were cultivated in 250 mL flasks with 50 mL minimal medium plus 20 mM TPA and 20 mM EG as carbon sources. All values represent the mean of three biological replicates, with error bars depicting the standard deviation from that mean.

FIG. 9. Strength of different promoter candidates in both RPET and E. coli strains. a, Schematic diagram of the replicating plasmid used for screening promoters and manipulating the native MEP pathway. b, Measurements of the promoter activity in RPET, based on fluorescence of a GFP+ reporter gene. Cultures were grown in minimal media with 20 mM TPA and EG as carbon sources; fluorescence was measured when cultures entered early stationary phase and was normalized to OD₆₀₀. C, Measurements of the promoter activity in E. coli. The GFP+ constructs were transformed into E. coli DH10B strain, and cultivated in LB medium. Fluorescence was measured when cultures entered early stationary phase and was normalized to OD₆₀₀. All values represent the mean of triplicate cultures, with error bars depicting the standard deviation from that mean.

FIG. 10. Cell growth and substrate consumption profile of WT RPET strain with cultivation on PET enzymatic hydrolysate. PET enzymatic hydrolysis was carried out by ICCG, a powerful variant of LCC, at 70° C. in a stirred-tank reactor. The resultant crude hydrolysate was centrifuged to remove uncatalyzed PET residue, and the clear supernatant was used as carbon feedstock without further purification. WT RPET cultured in glucose was used as inoculum. At each time point, a 200 μL sample of the cell suspension was taken to measure the cell density (OD), and the concentrations of TPA and EG in the supernatant were determined by HPLC. All values represent the mean of triplicate cultures, with error bars depicting the standard deviation from that mean.

FIG. 11. Evaluation of the PCA degradation pathway in RPET. a, The PCA cleavage pathways that have been identified in bacteria. 4CHMS, 4-carboxy-2-hydroxymuconate semialdehyde; 5CHMS, 5-carboxy-2-hydroxymuconate-6-semialdehyde; CM, 3-carboxy-cis, cis-muconate. b, The proposed PCA degradation pathways in RPET; the pcaHG operon was knocked out to confirm PCA degradation via hydroxyquinol (red X). c, A schematic of pcaHG knockout in RPET via homologous recombination. d, Comparisons of cell growth between WT and the ΔpcaHG knockout mutant with cultivation on 15 mM TPA or 15 PCA as sole carbon source. All values represent the mean of triplicate cultures, with error bars depicting the standard deviation from that mean.

FIG. 12. HPLC analysis of the lycopene production in RPET derived strains. All strains were cultivated in 250 mL flasks with 50 mL minimal medium plus 50 mM TPA and 50 mM EG as carbon sources. All values represent the mean of three biological replicates, with error bars depicting the standard deviation from that mean.

FIG. 13. Evaluation of the lycopene production in Strain S6 from different carbon sources. a, Cell growth of Strain S6 when cultivated on PET alkaline hydrolysate or on glucose. Strain S6 was inoculated into minimal medium with 10-fold diluted PET hydrolysate or 71.67 mM glucose (equivalent carbon content to the 10-fold diluted PET hydrolysate) as carbon source. b, Comparison of the lycopene production in the two different conditions. All values represent the mean of triplicate cultures, with error bars depicting the standard deviation from that mean.

FIG. 14. PET upcycling schematic.

FIG. 15. RPET (high salt-tolerant Rhodococcus)

FIG. 16. RPET TPA pathway elucidation. KO experiments to build the metabolic map.

FIG. 17. Muconate (MA) production to confirm the map.

FIG. 18. RPET pathway map for various products.

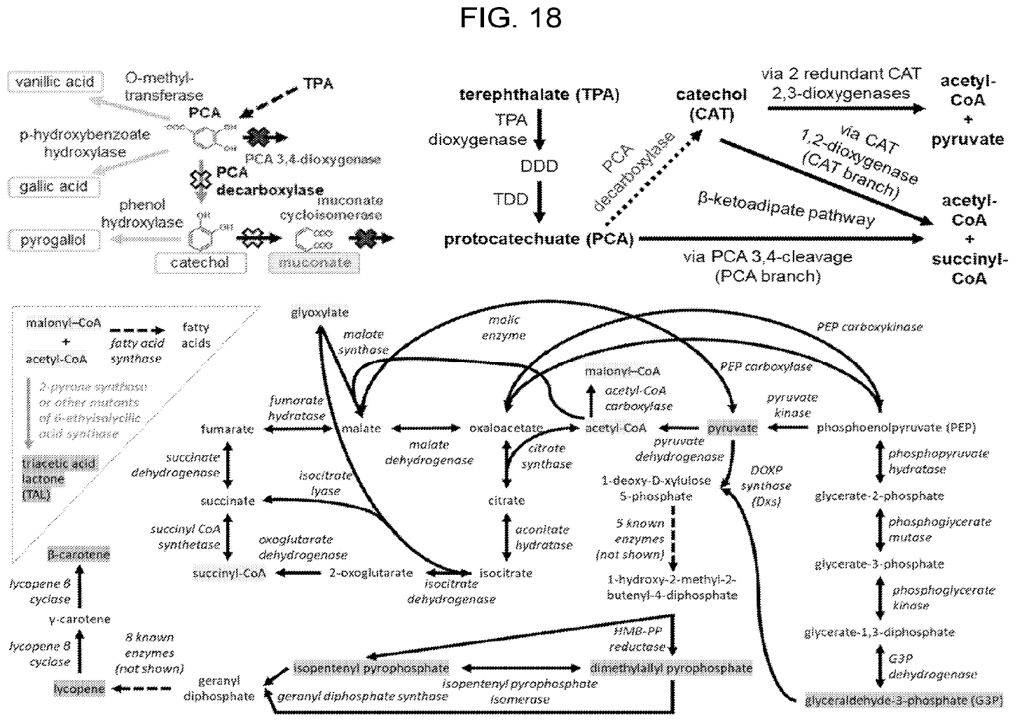

### DETAILED DESCRIPTION

The present disclosure is based, at least in part, on the discovery of microbes and TPA or EG degradation pathways for upcycling polyethylene terephthalate (PET) and increasing the efficiency of PET upcycling. TPA or EG degradation pathway(s) or other product formation pathway introduced can be native (from a Rhodococcus sp. strain) or non-native (from another microorganism). The present disclosure provides for re-designing the global plastic economy where plastic wastes are upcycled to produce high-value compounds rather than being discarded or simply recycled. To this end, here is provided an innovative strategy for polyethylene terephthalate (PET).

Many global companies are interested in PET upcycling, but have currently only technologies to chemically recycle PET instead of the hybrid technology such as chemical combined with biological, as described herein.

To overcome a critical limitation of relatively high process cost and the extremely low price of virgin PET, through strain screening, the discovered Rhodococcus strain (RPET) can grow well on the alkaline hydrolysis products of PET as the sole carbon source without any purification step.

Notably, this strain was able to tolerate and grow on a mixture of TPA and EG at extremely high concentrations (up to 0.3 M each, total 0.6 M) and high osmolarity resulting from alkaline hydrolysis and PH neutralization. Specifically, a simple depolymerization process led to a monomer mixture.

The resultant pH neutralized media supported RPET's growth without any purification and sterilization step except for their dilution to make monomer mixtures with necessary salts. In addition, many synthetic biology tools, developed for Rhodococcus opacus (related species), were functional in RPET, facilitating its engineering. Here is shown, the development of this novel system for waste PET valorization with PET conversion into carotenoids and muconate as two demonstration products.

An RPET strain, as described herein, can accumulate target product in an alkaline environment. An alkaline environment can be in a setting that is strongly basic or contains alkali components. This usually refers to an environment with a pH value higher than 7.0, since a pH below 7.0 is considered acidic. Here, the pH can be about 8.

### Polyethylene Terephthalate (PET)

The present disclosure provides for producing terephthalic acid (TPA) and ethylene glycol (EG) through alkaline hydrolysis of polyethylene terephthalate (PET) and biocatalysis to produce higher value products (compared to PET).

PET is a polyester of terephthalic acid (TPA) and ethylene glycol (EG). Due to its excellent physical properties, PET has been widely used in synthetic fibers and packaging materials. In 2015, annual global PET production reached 33 million tons, making PET the most commonly produced polyester worldwide. Because PET is not completely degraded by nature, it causes serious environmental problems such as the dissemination of microplastics in terrestrial ecosystems and the accumulation of waste plastics in the sea. However, biodegradable plastics having similar physical properties and economics to PET are not yet available. It is unlikely that PET production will be reduced in the near future, so PET recycling needs to be more stringent to reduce natural waste PET. Unrecycled PET waste causes serious environmental problems. To increase the PET recycling rate, the upcycling of PET into products that are higher value than PET is desired. As shown herein, various higher-value chemicals can be generated from PET monomers.

Of the various plastics, PET and polyethylene (PE) are the only plastics that are physically recycled, and recycled plastics are produced from waste plastics. Mechanical PET recycling has been around for decades, but this traditional recycling rate is lower than about 21% in the United States. This lower ratio seems to be mainly due to the lower quality and higher cost of recycled PET (e.g., $1.3-1.5/kg PET) compared to pure PET ($1.1-1.3/kg PET).

Polyethylene terephthalate (PET) represents significant global solid waste. PET chemical recycling has been an option to solve this global problem, but it has one main challenge: its relatively high process cost and the extremely low price of virgin PET. Improving the high cost and low economic feasibility of mechanical recycling functioning as downcycling, for example, blending mechanically recycled PET with lignin to produce carbon fibers, has been studied as an alternative application of mechanically recycled PET.

Another possible solution to address this issue is to upcycle waste PET rather than recycle it to generate the same PET, but typically results in low quality.

To overcome the problem of the downcycling of PET via mechanical recycling, chemical recycling, in which PET is depolymerized to its monomer and the monomers are repolymerized to PET, has been developed. Furthermore, a method for the chemical upcycling of waste PET into higher-value plastics by the chemical modification of PET and reinforcement with fiberglass was developed. Alternatively, once PET is chemically or biologically depolymerized into its monomers, especially TPA, the monomers can be biologically converted to another plastic monomer such as polyhydroxyalkanoate (PHA). However, the economic sustainability of this bioconversion process for PHA is still questionable. However, because of the high energy costs of the depolymerization of PET, the production of PET by chemical recycling or upcycling also has little to no economic benefit. Therefore, it is necessary to improve PET recycling economics through upcycling by converting monomers to products higher in value than PET.

Accordingly, the present disclosure provides for the development of an effective PET upcycling strategy. As described herein, PET upcycling can be achieved by depolymerizing PET into terephthalic acid (TPA) and ethylene glycol (EG) and biologically converting these monomers into value-added products.

Here is reported a new biological upcycling method that, when paired with a high-efficient PET depolymerization method, enables the valorization of plastic waste towards the sustainable production of high value-added compounds such as lycopene. By exploiting the metabolic versatility of RPET, many more valuable products (e.g., aromatics, organic acids, lipids and fuel molecules) could be sustainably produced through the upcycling of PET waste streams, contributing solutions to the challenges of the plastic pollution.

### Biological Valorization of Poly(Ethylene Terephthalate) Monomers for Upcycling Waste PET

Generally, waste valorization is the process of taking waste and changing it into constituent parts that can be utilized, and have value beyond the cost of the energy needed to process the transformation.

Here, is shown the biological valorization of PET monomers using a Rhodococcus strain (named RPET) to improve the economics of waste PET recycling and to develop effective PET upcycling strategies. For biological PET valorization, PET was depolymerized by chemical hydrolysis, and TPA and EG monomers were converted to a variety of higher-value chemicals (e.g., carotenoids and muconate).

By using various metabolically engineered whole-cell microbial catalysts, e.g., by introducing a TPA or EG degradation pathway or other product formation pathway into microbes, TPA or EG can be converted into higher-value aromatic or aromatic-derived chemicals, namely, protocatechuic acid (PCA), gallic acid (GA), pyrogallol, catechol, muconic acid (MA), and vanillic acid (VA), to be used for manufacturing pharmaceuticals, cosmetics, sanitizers, animal feeds, bioplastic monomers, and so on.

As shown herein, the novel system for waste PET valorization converts PET into carotenoids (e.g., lycopene) and muconate as two demonstration products is described herein.

Muconic acid (MA) is a high value-added dicarboxylic acid with conjugated double bonds, presenting three isomeric forms, i.e., cis, cis-MA, cis, trans-MA, and trans, trans-MA. Its production is garnering increased interest owing to its potential as a starting material for the synthesis of value-added products as well as by being a versatile monomer for the production of specialty polymers. The valorization of the three different isomers of MA into value-added chemicals such as adipic or terephthalic acids and MA polymers (see Khalil et al. Green Chem., 2020, 22, 1517-1541). Carotenoids are among the most abundant natural pigments available in nature. These pigments have received considerable attention because of their biotechnological applications and, more importantly, due to their potential beneficial uses in human healthcare, food processing, pharmaceuticals, and cosmetics.

The growth, culture, or incubation media comprises a carbon source which can comprise TPA and/or EG. For example, the carbon source may have about a 1:1, 1:2, 1:3, 1:4, 1:5, 1:6, 1:7, 1:8, 1:9, or 1:10 ratio of TPA to EG. As another example, the carbon source may have about a 1:1, 1:2, 1:3, 1:4, 1:5, 1:6, 1:7, 1:8, 1:9, or 1:10 ratio of EG to TPA. In some embodiments, the carbon source may have about a 1:1 ratio of TPA to EG. Recitation of each of these discrete values is understood to include ranges between each value. Recitation of each range is understood to include discrete values within the range.

The growth, culture, or incubation media comprises a carbon source which can comprise TPA and/or EG. The media can comprises about equimolar TPA and EG. In some embodiments, TPA and/or EG independently can be at a concentration of: between about 5 mM and 400 mM, between about 40 mM to 100 mM, between about 40 mM to 300 mM, or between about 150 to 300 mM. In some embodiments, TPA and/or EG independently can be at a concentration of: about 1 mM; about 2 mM; about 3 mM; about 4 mM; about 5 mM; about 6 mM; about 7 mM; about 8 mM; about 9 mM; about 10 mM; about 11 mM; about 12 mM; about 13 mM; about 14 mM; about 15 mM; about 16 mM; about 17 mM; about 18 mM; about 19 mM; about 20 mM; about 21 mM; about 22 mM; about 23 mM; about 24 mM; about 25 mM; about 26 mM; about 27 mM; about 28 mM; about 29 mM; about 30 mM; about 31 mM; about 32 mM; about 33 mM; about 34 mM; about 35 mM; about 36 mM; about 37 mM; about 38 mM; about 39 mM; about 40 mM; about 41 mM; about 42 mM; about 43 mM; about 44 mM; about 45 mM; about 46 mM; about 47 mM; about 48 mM; about 49 mM; about 50 mM; about 51 mM; about 52 mM; about 53 mM; about 54 mM; about 55 mM; about 56 mM; about 57 mM; about 58 mM; about 59 mM; about 60 mM; about 61 mM; about 62 mM; about 63 mM; about 64 mM; about 65 mM; about 66 mM; about 67 mM; about 68 mM; about 69 mM; about 70 mM; about 71 mM; about 72 mM; about 73 mM; about 74 mM; about 75 mM; about 76 mM; about 77 mM; about 78 mM; about 79 mM; about 80 mM; about 81 mM; about 82 mM; about 83 mM; about 84 mM; about 85 mM; about 86 mM; about 87 mM; about 88 mM; about 89 mM; about 90 mM; about 91 mM; about 92 mM; about 93 mM; about 94 mM; about 95 mM; about 96 mM; about 97 mM; about 98 mM; about 99 mM; about 100 mM; about 101 mM; about 102 mM; about 103 mM; about 104 mM; about 105 mM; about 106 mM; about 107 mM; about 108 mM; about 109 mM; about 110 mM; about 111 mM; about 112 mM; about 113 mM; about 114 mM; about 115 mM; about 116 mM; about 117 mM; about 118 mM; about 119 mM; about 120 mM; about 121 mM; about 122 mM; about 123 mM; about 124 mM; about 125 mM; about 126 mM; about 127 mM; about 128 mM; about 129 mM; about 130 mM; about 131 mM; about 132 mM; about 133 mM; about 134 mM; about 135 mM; about 136 mM; about 137 mM; about 138 mM; about 139 mM; about 140 mM; about 141 mM; about 142 mM; about 143 mM; about 144 mM; about 145 mM; about 146 mM; about 147 mM; about 148 mM; about 149 mM; about 150 mM; about 151 mM; about 152 mM; about 153 mM; about 154 mM; about 155 mM; about 156 mM; about 157 mM; about 158 mM; about 159 mM; about 160 mM; about 161 mM; about 162 mM; about 163 mM; about 164 mM; about 165 mM; about 166 mM; about 167 mM; about 168 mM; about 169 mM; about 170 mM; about 171 mM; about 172 mM; about 173 mM; about 174 mM; about 175 mM; about 176 mM; about 177 mM; about 178 mM; about 179 mM; about 180 mM; about 181 mM; about 182 mM; about 183 mM; about 184 mM; about 185 mM; about 186 mM; about 187 mM; about 188 mM; about 189 mM; about 190 mM; about 191 mM; about 192 mM; about 193 mM; about 194 mM; about 195 mM; about 196 mM; about 197 mM; about 198 mM; about 199 mM; about 200 mM; about 201 mM; about 202 mM; about 203 mM; about 204 mM; about 205 mM; about 206 mM; about 207 mM; about 208 mM; about 209 mM; about 210 mM; about 211 mM; about 212 mM; about 213 mM; about 214 mM; about 215 mM; about 216 mM; about 217 mM; about 218 mM; about 219 mM; about 220 mM; about 221 mM; about 222 mM; about 223 mM; about 224 mM; about 225 mM; about 226 mM; about 227 mM; about 228 mM; about 229 mM; about 230 mM; about 231 mM; about 232 mM; about 233 mM; about 234 mM; about 235 mM; about 236 mM; about 237 mM; about 238 mM; about 239 mM; about 240 mM; about 241 mM; about 242 mM; about 243 mM; about 244 mM; about 245 mM; about 246 mM; about 247 mM; about 248 mM; about 249 mM; about 250 mM; about 251 mM; about 252 mM; about 253 mM; about 254 mM; about 255 mM; about 256 mM; about 257 mM; about 258 mM; about 259 mM; about 260 mM; about 261 mM; about 262 mM; about 263 mM; about 264 mM; about 265 mM; about 266 mM; about 267 mM; about 268 mM; about 269 mM; about 270 mM; about 271 mM; about 272 mM; about 273 mM; about 274 mM; about 275 mM; about 276 mM; about 277 mM; about 278 mM; about 279 mM; about 280 mM; about 281 mM; about 282 mM; about 283 mM; about 284 mM; about 285 mM; about 286 mM; about 287 mM; about 288 mM; about 289 mM; about 290 mM; about 291 mM; about 292 mM; about 293 mM; about 294 mM; about 295 mM; about 296 mM; about 297 mM; about 298 mM; about 299 mM; about 300 mM; about 301 mM; about 302 mM; about 303 mM; about 304 mM; about 305 mM; about 306 mM; about 307 mM; about 308 mM; about 309 mM; about 310 mM; about 311 mM; about 312 mM; about 313 mM; about 314 mM; about 315 mM; about 316 mM; about 317 mM; about 318 mM; about 319 mM; about 320 mM; about 321 mM; about 322 mM; about 323 mM; about 324 mM; about 325 mM; about 326 mM; about 327 mM; about 328 mM; about 329 mM; about 330 mM; about 331 mM; about 332 mM; about 333 mM; about 334 mM; about 335 mM; about 336 mM; about 337 mM; about 338 mM; about 339 mM; about 340 mM; about 341 mM; about 342 mM; about 343 mM; about 344 mM; about 345 mM; about 346 mM; about 347 mM; about 348 mM; about 349 mM; about 350 mM; about 351 mM; about 352 mM; about 353 mM; about 354 mM; about 355 mM; about 356 mM; about 357 mM; about 358 mM; about 359 mM; about 360 mM; about 361 mM; about 362 mM; about 363 mM; about 364 mM; about 365 mM; about 366 mM; about 367 mM; about 368 mM; about 369 mM; about 370 mM; about 371 mM; about 372 mM; about 373 mM; about 374 mM; about 375 mM; about 376 mM; about 377 mM; about 378 mM; about 379 mM; about 380 mM; about 381 mM; about 382 mM; about 383 mM; about 384 mM; about 385 mM; about 386 mM; about 387 mM; about 388 mM; about 389 mM; about 390 mM; about 391 mM; about 392 mM; about 393 mM; about 394 mM; about 395 mM; about 396 mM; about 397 mM; about 398 mM; about 399 mM; or about 400 mM. Recitation of each of these discrete values is understood to include ranges between each value. Recitation of each range is understood to include discrete values within the range.

As described herein, the Rhodococcus sp. strain grown using PET hydrolysis products as a carbon source can generate significant biomass (e.g., g DCW/g PET). In some embodiments, the Rhodococcus sp. strain can yield between 10% and 300% biomass per used PET. In some embodiments, the Rhodococcus sp. strain can yield biomass in about 1%; about 2%; about 3%; about 4%; about 5%; about 6%; about 7%; about 8%; about 9%; about 10%; about 11%; about 12%; about 13%; about 14%; about 15%; about 16%; about 17%; about 18%; about 19%; about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; about 40%; about 41%; about 42%; about 43%; about 44%; about 45%; about 46%; about 47%; about 48%; about 49%; about 50%; about 51%; about 52%; about 53%; about 54%; about 55%; about 56%; about 57%; about 58%; about 59%; about 60%; about 61%; about 62%; about 63%; about 64%; about 65%; about 66%; about 67%; about 68%; about 69%; about 70%; about 71%; about 72%; about 73%; about 74%; about 75%; about 76%; about 77%; about 78%; about 79%; about 80%; about 81%; about 82%; about 83%; about 84%; about 85%; about 86%; about 87%; about 88%; about 89%; about 90%; about 91%; about 92%; about 93%; about 94%; about 95%; about 96%; about 97%; about 98%; about 99%; about 100%; about 101%; about 102%; about 103%; about 104%; about 105%; about 106%; about 107%; about 108%; about 109%; about 110%; about 111%; about 112%; about 113%; about 114%; about 115%; about 116%; about 117%; about 118%; about 119%; about 120%; about 121%; about 122%; about 123%; about 124%; about 125%; about 126%; about 127%; about 128%; about 129%; about 130%; about 131%; about 132%; about 133%; about 134%; about 135%; about 136%; about 137%; about 138%; about 139%; about 140%; about 141%; about 142%; about 143%; about 144%; about 145%; about 146%; about 147%; about 148%; about 149%; about 150%; about 151%; about 152%; about 153%; about 154%; about 155%; about 156%; about 157%; about 158%; about 159%; about 160%; about 161%; about 162%; about 163%; about 164%; about 165%; about 166%; about 167%; about 168%; about 169%; about 170%; about 171%; about 172%; about 173%; about 174%; about 175%; about 176%; about 177%; about 178%; about 179%; about 180%; about 181%; about 182%; about 183%; about 184%; about 185%; about 186%; about 187%; about 188%; about 189%; about 190%; about 191%; about 192%; about 193%; about 194%; about 195%; about 196%; about 197%; about 198%; about 199%; about 200%; about 201%; about 102%; about 203%; about 204%; about 205%; about 206%; about 207%; about 208%; about 209%; about 210%; about 211%; about 212%; about 213%; about 214%; about 215%; about 216%; about 217%; about 218%; about 219%; about 220%; about 221%; about 222%; about 223%; about 224%; about 225%; about 226%; about 227%; about 228%; about 229%; about 230%; about 231%; about 232%; about 233%; about 234%; about 235%; about 236%; about 237%; about 238%; about 239%; about 240%; about 241%; about 242%; about 243%; about 244%; about 245%; about 246%; about 247%; about 248%; about 249%; about 250%; about 251%; about 252%; about 253%; about 254%; about 255%; about 256%; about 257%; about 258%; about 259%; about 260%; about 261%; about 262%; about 263%; about 264%; about 265%; about 266%; about 267%; about 268%; about 269%; about 270%; about 271%; about 272%; about 273%; about 274%; about 275%; about 276%; about 277%; about 278%; about 279%; about 280%; about 281%; about 282%; about 283%; about 284%; about 285%; about 286%; about 287%; about 288%; about 289%; about 290%; about 291%; about 292%; about 293%; about 294%; about 295%; about 296%; about 297%; about 298%; about 299%; or about 300%. Recitation of each of these discrete values is understood to include ranges between each value. Recitation of each range is understood to include discrete values within the range.

The RPET strain is grown in media comprising Na⁺ or K⁺. In some embodiments, the Na⁺ or K⁺ concentration is between about 80 mM to 200 mM or no more than about 200 mM Na⁺ or K⁺. In some embodiments, the Na⁺ or K⁺ concentration is about 1 mM; about 2 mM; about 3 mM; about 4 mM; about 5 mM; about 6 mM; about 7 mM; about 8 mM; about 9 mM; about 10 mM; about 11 mM; about 12 mM; about 13 mM; about 14 mM; about 15 mM; about 16 mM; about 17 mM; about 18 mM; about 19 mM; about 20 mM; about 21 mM; about 22 mM; about 23 mM; about 24 mM; about 25 mM; about 26 mM; about 27 mM; about 28 mM; about 29 mM; about 30 mM; about 31 mM; about 32 mM; about 33 mM; about 34 mM; about 35 mM; about 36 mM; about 37 mM; about 38 mM; about 39 mM; about 40 mM; about 41 mM; about 42 mM; about 43 mM; about 44 mM; about 45 mM; about 46 mM; about 47 mM; about 48 mM; about 49 mM; about 50 mM; about 51 mM; about 52 mM; about 53 mM; about 54 mM; about 55 mM; about 56 mM; about 57 mM; about 58 mM; about 59 mM; about 60 mM; about 61 mM; about 62 mM; about 63 mM; about 64 mM; about 65 mM; about 66 mM; about 67 mM; about 68 mM; about 69 mM; about 70 mM; about 71 mM; about 72 mM; about 73 mM; about 74 mM; about 75 mM; about 76 mM; about 77 mM; about 78 mM; about 79 mM; about 80 mM; about 81 mM; about 82 mM; about 83 mM; about 84 mM; about 85 mM; about 86 mM; about 87 mM; about 88 mM; about 89 mM; about 90 mM; about 91 mM; about 92 mM; about 93 mM; about 94 mM; about 95 mM; about 96 mM; about 97 mM; about 98 mM; about 99 mM; about 100 mM; about 101 mM; about 102 mM; about 103 mM; about 104 mM; about 105 mM; about 106 mM; about 107 mM; about 108 mM; about 109 mM; about 110 mM; about 111 mM; about 112 mM; about 113 mM; about 114 mM; about 115 mM; about 116 mM; about 117 mM; about 118 mM; about 119 mM; about 120 mM; about 121 mM; about 122 mM; about 123 mM; about 124 mM; about 125 mM; about 126 mM; about 127 mM; about 128 mM; about 129 mM; about 130 mM; about 131 mM; about 132 mM; about 133 mM; about 134 mM; about 135 mM; about 136 mM; about 137 mM; about 138 mM; about 139 mM; about 140 mM; about 141 mM; about 142 mM; about 143 mM; about 144 mM; about 145 mM; about 146 mM; about 147 mM; about 148 mM; about 149 mM; about 150 mM; about 151 mM; about 152 mM; about 153 mM; about 154 mM; about 155 mM; about 156 mM; about 157 mM; about 158 mM; about 159 mM; about 160 mM; about 161 mM; about 162 mM; about 163 mM; about 164 mM; about 165 mM; about 166 mM; about 167 mM; about 168 mM; about 169 mM; about 170 mM; about 171 mM; about 172 mM; about 173 mM; about 174 mM; about 175 mM; about 176 mM; about 177 mM; about 178 mM; about 179 mM; about 180 mM; about 181 mM; about 182 mM; about 183 mM; about 184 mM; about 185 mM; about 186 mM; about 187 mM; about 188 mM; about 189 mM; about 190 mM; about 191 mM; about 192 mM; about 193 mM; about 194 mM; about 195 mM; about 196 mM; about 197 mM; about 198 mM; about 199 mM; about 200 mM; about 201 mM; about 202 mM; about 203 mM; about 204 mM; about 205 mM; about 206 mM; about 207 mM; about 208 mM; about 209 mM; about 210 mM; about 211 mM; about 212 mM; about 213 mM; about 214 mM; about 215 mM; about 216 mM; about 217 mM; about 218 mM; about 219 mM; about 220 mM; about 221 mM; about 222 mM; about 223 mM; about 224 mM; about 225 mM; about 226 mM; about 227 mM; about 228 mM; about 229 mM; about 230 mM; about 231 mM; about 232 mM; about 233 mM; about 234 mM; about 235 mM; about 236 mM; about 237 mM; about 238 mM; about 239 mM; about 240 mM; about 241 mM; about 242 mM; about 243 mM; about 244 mM; about 245 mM; about 246 mM; about 247 mM; about 248 mM; about 249 mM; about 250 mM; about 251 mM; about 252 mM; about 253 mM; about 254 mM; about 255 mM; about 256 mM; about 257 mM; about 258 mM; about 259 mM; about 260 mM; about 261 mM; about 262 mM; about 263 mM; about 264 mM; about 265 mM; about 266 mM; about 267 mM; about 268 mM; about 269 mM; about 270 mM; about 271 mM; about 272 mM; about 273 mM; about 274 mM; about 275 mM; about 276 mM; about 277 mM; about 278 mM; about 279 mM; about 280 mM; about 281 mM; about 282 mM; about 283 mM; about 284 mM; about 285 mM; about 286 mM; about 287 mM; about 288 mM; about 289 mM; about 290 mM; about 291 mM; about 292 mM; about 293 mM; about 294 mM; about 295 mM; about 296 mM; about 297 mM; about 298 mM; about 299 mM; or about 300 mM. Recitation of each of these discrete values is understood to include ranges between each value. Recitation of each range is understood to include discrete values within the range.

### PET Hydrolysis

Generally, hydrolysis of PET is a chemical reaction carried out in the presence of a catalyst, usually a metallic salt soluble in water, and the mechanism shows that the metal ion attacks the C═O bond producing an electrolytic destabilization in the neighboring bonds, followed by cleavage of the polymer chain. Alkaline hydrolysis of PET is generally carried out with the use of an aqueous alkaline solution of NaOH or KOH. PET hydrolysis can also include enzymatic hydrolysis or other chemical hydrolysis methods of PET.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

Here it was shown that a synthetic operon crtEBI under the control of the arabinose-inducible promoter pBAD on a shuttle plasmid and transformed RPET strain, could be utilized to accumulate lycopene. Any native or transgene can be introduced into the RPET strain to accumulate molecules of interest. It was also shown that knocking out certain genes or operons in specific pathways (e.g., pcaHG, the operon which putatively encodes the PCA 3,4-cleavage pathway) can increase accumulation of specific products or precursors. As such, synthetic genes or operons can comprise or be derived from native genes or transgenic genes (transgenes).

By using various metabolically engineered whole-cell microbial catalysts, e.g., by introducing a TPA or EG degradation pathway or other product formation pathway into microbes, TPA or EG can be converted into higher-value aromatic or aromatic-derived chemicals, namely, protocatechuic acid (PCA), gallic acid (GA), pyrogallol, catechol, muconic acid (MA), and vanillic acid (VA), to be used for manufacturing pharmaceuticals, cosmetics, sanitizers, animal feeds, bioplastic monomers, and so on. Here, the engineering can be as described in DeLorenzo et al. ACS Synth. Biol. 2021, 10, 786-798 unless noted otherwise.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments (e.g., comprising polynucleotides) are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine (RNA only) | U | A |
| G | Guanidine | G | C |
| C | Cytidine | C | G |
| Y | pYrimidine | C T | R |
| R | puRine | A G | Y |
| S | Strong (3 H bonds) | G C | S* |
| W | Weak (2 H bonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, artificial DNA construct, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted or deleted from a DNA or RNA sequence of an organism's genome. Point mutations have a variety of effects on the downstream protein product-consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6 (log₁₀[Na⁺])+0.41 (fraction G/C content)−0.63 (% formamide)−(600/l). Furthermore, the Tₘ Of a DNA:DNA hybrid is decreased by 1-1.5ºC for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged | |
| (Acidic): | D E |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, activity, signals, expression, or function can be modulated (e.g., reduced, eliminated, or enhanced) using genome editing (e.g., upregulate, downregulate, silence, overexpress, underexpress, express (e.g., transgenic expression), knock in, knock out, knockdown) based on the knowledge of the synthetic pathways of the microorgamism (e.g., RPET or other microorganism having a synthetic pathway to accumulate products from PET hydrolysate). Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools to target cells by the removal or addition of expression or signals (e.g., activate (e.g., CRISPRa), upregulate, downregulate a protein, enzyme, etc.).

For example, the methods as described herein can comprise a method for altering a target polynucleotide sequence in a cell comprising contacting the polynucleotide sequence with a clustered regularly interspaced short palindromic repeats-associated (Cas) protein.

### Genes, Pathways, and Target Products

As described herein, the Rhodococcus sp. strain can be engineered to accumulate target products.

There are various ways to accumulate target products, target product derivatives, or target product precursors. For example, the pathways described herein can be exploited, regulated, or modulated. Modulation can be upregulating, downregulating over-expressing, under-expressing, knocking in, knocking down, knocking out, inserting synthetic pathways, etc. For example, the following enzymes or genes encoding the enzymes in the RPET pathway can be regulated or modulated: 1-deoxy-d-xylulose 5-phosphate reductoisomerase (dxr); 4-diphosphocytidyl-2C-methyl-D-erythritol synthase (ispD); isopentenyl monophosphate kinase (ispE); 2-C-methyl-D-erythritol 2,4-cyclodiphosphate synthase (ispF); 4-hydroxy-3-methylbut-2-en-1-yl diphosphate synthase (ispG); pyrophosphate synthase; geranylgeranyl diphosphate synthase; phytoene synthase; phytoene desaturase; zeta-carotene isomerase; zeta-carotene desaturase; carotenoid isomerase; 2-pyrone synthase; 6-ethylsalycilic acid synthase; acetyl-CoA carboxylase; aconitate hydratase; CAT 1,2-dioxygenase activity; CAT 2,3-dioxygenase activity; citrate synthase; DOXP synthase (Dxs); fatty acid synthase; fumarate hydratase; glyceraldehyde-3-phosphate (G3P) dehydrogenase; geranyl diphosphate synthase; HMB-PP reductase; isocitrate dehydrogenase; isocitrate lyase; isopentenyl pyrophosphate isomerase; lycopene β cyclase; malate dehydrogenase; malate synthase; malic enzyme; muconate cycloisomerase activity; O-methyl transferase activity; oxoglutarate dehydrogenase; PCA 3,4-dioxygenase activity; PCA decarboxylase; PEP carboxykinase; PEP carboxylase; phenol hydroxylase; phosphoglycerate kinase; phosphoglycerate mutase; phosphopyruvate hydratase; p-hydroxybenzoate hydroxylase; pyruvate dehydrogenase; pyruvate kinase; succinate dehydrogenase; succinyl CoA synthetase; terephthalic acid (TPA) dioxygenase or combinations thereof.

Other enzymes of target product formation pathways can be AceE, Pyruvate dehydrogenase E1 component; AcnA, Aconitate hydratase; AlkK, Acyl-CoA synthetase; AroY, Protocatechuate decarboxylase; CatA, Catechol 1,2-dioxygenase; CatBC, Muconate cycloisomerase 1/Muconolactone Delta-isomerase; Eno, Enolase; ER, Enoate reductase; AccA, Acetyl-CoA carboxylase; FabAZ, 3-hydroxydecanoyl-[acyl-carrier-protein] dehydratase/3-hydroxyacyl-[acyl-carrier-protein] dehydratase FabZ; FabBF, 3-oxoacyl-[acyl-carrier-protein] synthase 1/3-oxoacyl-[acyl-carrier-protein] synthase 2; FabD, Malonyl CoA-acyl carrier protein transacylase; FabG, 3-oxoacyl-[acyl-carrier-protein] reductase; FabH, 3-oxoacyl-ACP synthase; FabIV, Enoyl-[acyl-carrier-protein] reductase [NADH]; GalB, 4-oxalmesaconate hydratase; GalC, 4-carboxy-4-hydroxy-2-oxoadipic acid aldolase; GalD, 4-oxalomesaconate tautomerase; Gcl, Glyoxylate carboligase; GIcDEF, Glycolate oxidase, putative FAD-linked subunit/Glycolate oxidase, putative FAD-binding subunit/; gltA, Citrate synthase; GIxR, Tartronate semialdehyde reductase; HsoMT, Catechol O-methyltransferase; Hyi, Hydroxypyruvate isomerase; Icd, Isocitrate dehydrogenase [NADP]; LigAB, Type II extradiol dioxygenases/protocatechuate 4,5-dioxygenase; LigC, 4-carboxy-2-hydroxymuconate-6-semialdehyde dehydrogenase; Ligl, 2-pyrone-4,6-dicarboxylate hydrolase; Lpdc, Gallate decarboxylase; Mcr, malonyl-CoA reductase; Mdh, Probable malate dehydrogenase; PcaB, 3-carboxy-cis,cis-muconate cycloisomerase; PcaC, 4-carboxymuconolactone decarboxylase; PcaD, 3-oxoadipate enol-lactonase 2; PcaF, 3-oxoadipyl-CoA thiolase; PcaHG, Protocatechuate 3,4-dioxygenase beta chain/Protocatechuate 3,4-dioxygenase alpha chain; PcalJ, 3-oxoadipate CoA-transferase; PedEH, PQQ-dependent dehydrogenase; Pedl, Aldehyde dehydrogenase; PhaC, Poly(3-hydroxyalkanoate) polymerase 2; PhaG, (R)-3-hydroxydecanoyl-ACP:CoA transacylase; PhaJ, (R)-specific enoyl-CoA hydratase; AceA-D, Isocitrase; PobA, p-hydroxybenzoate hydroxylase; PP_0897, Fumarate hydratase class I; PP_4300, Putative hydroxypyruvate reductase; PraA, Protocatechuate 2,3-dioxygenase; PraH, 5-carboxy-2-hydroxymuconate-6-semialdehyde decarboxylase; PykAF, Pyruvate kinase; SdhB, Succinate dehydrogenase iron-sulfur subunit; SucAB, Oxoglutarate dehydrogenase (succinyl-transferring)/Dihydrolipoyllysine-residue succinyltransferase component of 2-oxoglutarate dehydrogenase complex; SucCD, Succinate—CoA ligase [ADP-forming] subunit beta/; TphAabc, Terephthalate 1,2-dioxygenase; TphB, 4-hydroxythreonine-4-phosphate dehydrogenase; TpiABC, Triosephosphate isomerase/Small transmembrane protein of the aromatic acids transporter; XyIG, 2-hydroxymuconic semialdehyde dehydrogenase; XyIH, 2-hydroxymuconate tautomerase; XylI, 4-oxalocrotonate decarboxylase; XyIJ, 2-oxopent-4-enoate hydratase; XyIK, 4-hydroxy-2-oxovalerate aldolase; or XyIQ, Acetaldehyde dehydrogenase.

As another example, in Corynebacterium glutamicum, it was reported that the overexpression of prenyltransferase—(CrtE), phytoene synthase—(CrtB), and phytoene desaturase—(CrtI) encoding genes significantly improved the flux from the precursor molecules isopentenyl pyrophosphate (IPP) and dimethylallyl pyrophosphate (DMAPP) to lycopene. Thus, the following enzymes or genes encoding the enzymes can be introduced to an RPET strain, regulated, or modulated in RPET: geranylgeranyl diphosphate synthase (CrtE); phytoene synthase (CrtB); phytoene desaturase (CrtI); lycopene beta-cyclase (CrtL-b); or combinations thereof. As another example, the following enzymes or genes encoding the enzymes in the 2-methylerythritol 4-phosphate (MEP) pathway can be regulated or modulated: 1-deoxyxylulose-5-phosphate synthase (dxs); 1-deoxy-d-xylulose 5-phosphate reductoisomerase (dxr); 4-diphosphocytidyl-2C-methyl-D-erythritol synthase (ispD); isopentenyl monophosphate kinase (ispE); 2-C-methyl-D-erythritol 2,4-cyclodiphosphate synthase (ispF); 4-hydroxy-3-methylbut-2-en-1-yl diphosphate synthase (ispG); 4-hydroxy-3-methylbut-2-enyl diphosphate reductase (ispH); isopentenyl pyrophosphate isomerase (idi); or combinations thereof.

As described herein, an RPET strain can be engineered to produce or modulate (e.g., increase or decrease) accumulation or production of a target product or a target product precursors. In some embodiments, the target product can be derived from or the target product or target product precursors can be: 1-hydroxy-2-methyl-2-butenyl-4-diphosphate; 1-deoxy-D-xylulose-5-phosphate; 2-oxoglutarate; acetyl-CoA; catechol (CAT); citrate; dimethylallyl pyrophosphate; fatty acids; fumarate; gallic acid (GA); geranyl diphosphate; glyceraldehyde-3-phosphate (G3P); glycerate-1,3-diphosphate; glycerate-2-phosphate; glycerate-3-phosphate; glyoxylate; isocitrate; isopentyl pyrophosphate; lycopene; malate; 10 malonyl-CoA; muconate or muconic acid (MA); oxaloacetate; phosphophenylpyruvate (PEP); protocatechuate or protocatechuic acid (PCA); pyrogallol; pyruvate; succinate; succinyl-CoA; triacetic acid lactone (TAL); vanillic acid (VA); β-carotene; or γ-carotene; or combinations thereof. In some embodiments, the target product can be derived from or the target product or target product precursors can be 4-carboxy-2-hydroxymuconate semialdehyde (4CHMS); 5-carboxy-2-hydroxymuconate-6-semialdehyde (5CHMS); or 3-carboxy-cis, cis-muconate (CM); or combinations thereof. In some embodiments, the target product can be derived from or the target product or target product precursors can be 2-phosphoglycerate; acetyl-CoA; citrate; farnesyl pyrophosphate (FPP); fumarate; geranyl pyrophosphate (GPP); glyceraldehyde-3-phosphate (G3P); glyoxylate; isocitrate; lycopene; malate; oxaloacetate; pyruvate (PYR); succinate; succinate; succinyl-CoA; α-ketogluterate; β-carotene; or combinations thereof. In some embodiments, the target product can be derived from or the target product or target product precursors can be 1-deoxy-D-xylulose-5-phosphate (DXP); 1-hydroxy-2-methyl-2-(E)-butenyl-4-diphosphate (HMB-PP); 2-C-methyl-D-erythritol 4-phosphate (MEP); 2-C-methyl-D-erythritol-2,4-cyclo-diphosphate (MEC); 4-diphosphocytidyl-2-C-methyl-D-erythritol (CDP-ME); 4-diphosphocytidyl-2-C-methyl-D-erythritol 2-phosphate (CDP-MEP); dimethylallyl diphosphate (DMAPP); isopentenyl pyrophosphate (IPP); or combinations thereof. In some embodiments, the target product or target product precursors can be protocatechuic acid (PCA), gallic acid (GA), pyrogallol, catechol, muconic acid (MA) (e.g., cis, cis-MA, cis, trans-MA and trans, trans-MA), or vanillic acid (VA).

Carotenoids or muconate/muconate-derived products can be as described in Khalil, Green Chem., 2020, 22, 1517. PET degradation products can also be as described in Qi, Microorganisms 2022, 10, 39.

For example, types of Muconate (Muconic Acid) can be cis, cis muconic acid (ccMA), cis, trans-muconic acid (ctMA), or trans, trans-muconic acid (ttMA). High-value muconate-derived products can include Adipic acid, lactones (muconolactone, dilactone and ε-caprolactam), terephthalic acid and terephthalates, dialkylmuconates, or polymers.

As another example, carotenoids can be 2,2′-Diketospirilloxanthin, 3′-Hydroxyechinenone, 3-OH-Canthaxanthin (Adonirubin or Phoenicoxanthin), Actinioerythrin, Alloxanthin, Apo-2-lycopenal, Apo-6′-lycopenal, Astacein, Astacene, Astaxanthin, Azafrinaldehyde, Bacterioruberin, Bixin, C.p. 450, C.p. 473, Canthaxanthin (Aphanicin or Chlorellaxanthin), Capsanthin, Capsorubin, Citranaxanthin, Citroxanthin, Crocetin, Crocin (Digentiobiosyl), Crustaxanthin, Cryptocapsin, Cryptomonaxanthin, Decaprenoxanthin, Diadinoxanthin, Echinenone, Eschscholtzxanthin, Eschscholtzxanthone, Flexixanthin, Foliachrome, Fucoxanthin, Gazaniaxanthin, Hexahydrolycopene, Hopkinsiaxanthin, Hydroxyspheriodenone, Isofucoxanthin, Loroxanthin, Lutein, Luteoxanthin, Lycopene, Lycopersene, Lycoxanthin, Methyl apo-6′-lycopenoate, Mutatoxanthin, Neochrome, Nonaprenoxanthin, OH-Chlorobactene, Okenone, Oscillaxanthin, Paracentrone, Pectenolone, Pectenoxanthin, Peridinin, Phleixanthophyll, Phoeniconone (Dehydroadonirubin), Phoenicopterone, Physalien, Phytofluene, Pyrrhoxanthininol, Rhodopin, Rhodopinal, Rhodopinol (Warmingol), Rhodovibrin, Rhodoxanthin, Rubixanthone, Saproxanthin, Semi-α-carotenone, Semi-β-carotenone, Sintaxanthin, Siphonaxanthin, Siphonein, Spheroidene, Tangeraxanthin, Torularhodin methyl ester, Torularhodin, Torularhodinaldehyde, Torulene, Triphasiaxanthin, Trollichrome, Vaucheriaxanthin, Warmingone, Zeaxanthin, Zeaxanthin furanoxide, α-Carotene, α-Zeacarotene, β-Apo-2′-carotenal, β-Carotene, β-Carotenone, γ-Carotene, δ-Carotene, ε-Carotene, or ζ-Carotene. As another example, carotenoids can be hydrocarbons (e.g., Hexahydrolycopene, Lycopene, Lycopersene, Phytofluene, Torulene, α-Carotene, α-Zeacarotene, β-Carotene, γ-Carotene, δ-Carotene, ε-Carotene, ζ-Carotene); alcohols (e.g., Alloxanthin, Bacterioruberin, Crustaxanthin, Cryptomonaxanthin, Cynthiaxanthin, Gazaniaxanthin, Loroxanthin, Lutein, Lycoxanthin, OH-Chlorobactene, Pectenoxanthin, Rhodopin, Rhodopinol (Warmingol), Saproxanthin, Zeaxanthin); glycosides (e.g., Oscillaxanthin, Phleixanthophyll), ethers (e.g., Rhodovibrin, Spheroidene); epoxides (e.g., Citroxanthin, Diadinoxanthin, Foliachrome, Luteoxanthin, Mutatoxanthin, Neochrome, Trollichrome, Vaucheriaxanthin, Zeaxanthin furanoxide); aldehydes (e.g., Rhodopinal, Torularhodinaldehyde, Warmingone); acids or acid esters (e.g., Torularhodin, Torularhodin methyl ester); ketones (e.g., 2,2′-Diketospirilloxanthin, 3′-Hydroxyechinenone, 3-OH-Canthaxanthin (Adonirubin or Phoenicoxanthin), Astacene, Astaxanthin, Canthaxanthin (Aphanicin or Chlorellaxanthin), Capsanthin, Capsorubin, Cryptocapsin, Echinenone, Flexixanthin, Hydroxyspheriodenone, Okenone, Pectenolone, Phoeniconone (Dehydroadonirubin), Phoenicopterone, Rubixanthone, Siphonaxanthin); esters of alcohols (e.g., Astacein, Fucoxanthin, Isofucoxanthin, Physalien, Siphonein); apocarotenoids (e.g., Apo-2-lycopenal, Apo-6′-lycopenal, Azafrinaldehyde, Bixin, Citranaxanthin, Crocetin, Crocetinsemialdehyde, Crocin (Digentiobiosyl), Hopkinsiaxanthin, Methyl apo-6′-lycopenoate, Paracentrone, Sintaxanthin, β-Apo-2′-carotenal); nor- and seco-carotenoids (e.g., Actinioerythrin, Peridinin, Pyrrhoxanthininol, Semi-α-carotenone, Semi-β-carotenone, Triphasiaxanthin); retro-carotenoids and retro-apo-carotenoids (e.g., Eschscholtzxanthin, Eschscholtzxanthone, Rhodoxanthin, Tangeraxanthin); or higher carotenoids (e.g., Bacterioruberin, C.p. 450, C.p. 473, Decaprenoxanthin, Nonaprenoxanthin).

High-value target products/intermediates can be as described in Dissanayake, Front. Bioeng. 2021 Biotechnol. 9:656465. For example, Polyethylene terephthalate; Bis(2-Hydroxyethyl) terephthalate; Ethylene glycol; Terephthalic acid; Sodium terephthalate; (3S,4R)-3,4-Dihydroxy-1,5-cyclohexadiene-1,4-dicarboxylic acid; Protocatechuate; (Z)-(E)-4-Formylmethylidene-2-hydroxy-2-pentenedioate; 4-Carboxy-2-hydroxymuconate-semialdehyde; 2-Pyrone-4,6-dicarboxylic acid; 4-Oxalomesaconic acid (enol form); 4-Oxalomesaconic acid (Keto form); 4-carboxy-4-hydroxy-2-oxoadipic acid; Pyruvate; β-Carboxy-cic, cis-mucinic acid; Y-Carboxymuconolactone; Muconolactone; β-Ketoadipic acid; 3-oxoadipyl-CoA; Succinyl-CoA; Acetyl-CoA; 5-Carboxy-2-hydroxymuconate-semialdehyde; 2-Hydroxymuconate-semialdehyde; 4-Oxalocrotonic acid (enol form); 4-Oxalocrotonic acid (keto form); 4-Hydroxy-2-oxovaleric acid/2-Oxo-4-pentenoate; 4-Hydroxy-2-oxovaleric acid; Acetaldehyde; Glycolaldehyde; Glycolate; Glyoxalate; Tartronate semialdehyde; Hydroxypyruvate; Glycerate; 2-phosphoglycerate; Phosphoenolpyruvate; Gallic acid; Pyrogallol; Vanillin; Catechol; cis, cis-muconate; Malonyl-CoA; Malonyl-ACP; Acetoacyl-ACP; 3-Ketoacyl-ACP; (R)-3-Hydroxyacyl-ACP; Enoyl-ACP; Acyl-ACP; Malondialdehyde; 3-Hydroxypropionic acid; (R)-3-Hydroxyfatty acid; (R)-3-Hydroxyacyl-CoA; Medium chain length polyhydroxyalkanoate (mcl-PHA); 2-Trans-Enoyl—CoA; Citrate; Isocitrate; α-Ketoglutarate; Succinyl-CoA; Fumarate; Malate; Oxaloacetate; or Adipic acid.

### Rhodococcus

Rhodococcus is a genus of aerobic, nonsporulating, nonmotile Gram-positive bacteria closely related to Mycobacterium and Corynebacterium. While a few species are pathogenic, most are benign, and have been found to thrive in a broad range of environments, including soil, water, and eukaryotic cells. Some species have large genomes, including the 9.7 megabasepair genome (67% G/C) of Rhodococcus sp. RHA1.

Strains of Rhodococcus are important owing to their ability to catabolize a wide range of compounds and produce bioactive steroids, acrylamide, and acrylic acid, and their involvement in fossil fuel biodesulfurization. This genetic and catabolic diversity is not only due to the large bacterial chromosome, but also to the presence of three large linear plasmids. Rhodococcus is also an experimentally advantageous system owing to a relatively fast growth rate and simple developmental cycle, but is not well characterized. Example species of Rhodococcus can include Rhodococcus aerolatus Hwang et al. 2015; Rhodococcus aetherivorans Goodfellow et al. 2004; Rhodococcus agglutinans Guo et al. 2015; Rhodococcus aurantiacus (ex Tsukamura and Mizuno, 1971) Tsukamura and Yano, 1985, nom. rev.; Rhodococcus artemisiae Zhao et al. 2012; Rhodococcus baikonurensis Li, et al., 2004; Rhodococcus biphenylivorans Su et al. 2015; Rhodococcus boritolerans; Rhodococcus equi (Magnusson, 1923) Goodfellow and Alderson, 1977; Rhodococcus canchipurensis Nimaichand et al. 2013; Rhodococcus cerastii Kämpfer et al. 2013; Rhodococcus cercidiphylli Li et al. 2012; Rhodococcus coprophilus Rowbotham and Cross, 1979; Rhodococcus corynebacterioides (Serrano, et al., 1972) Yassin and Schaal, 2005 (synonym: Nocardia corynebacterioides (Serrano et al. 1972); Rhodococcus defluvii Kämpfer et al. 2014; Rhodococcus electrodiphilus Ramaprasad et al., 2018; Rhodococcus enclensis Dastager et al., 2014; Rhodococcus erythropolis (Gray and Thornton, 1928) Goodfellow and Alderson, 1979; Rhodococcus fascians (Tilford 1936) Goodfellow 1984 (synonym: Rhodococcus luteus (ex Sohngen 1913) Nesterenko et al. 1982); Rhodococcus globerulus Goodfellow, et al., 1985; Rhodococcus gordoniae Jones, et al., 2004; Rhodococcus hoagii Kämpfer et al. 2014; Rhodococcus imtechensis Ghosh et al. 2006; Rhodococcus jialingiae Wang et al. 2010; Rhodococcus jostii Takeuchi, et al., 2002. Identified as producing a lignin digesting enzyme, it was the first isolated from a bacterium rather than a fungus; Rhodococcus koreensis Yoon, et al., 2000; Rhodococcus kroppenstedtii Mayilraj, et al., 2006; Rhodococcus kunmingensis Wang et al., 2008; Rhodococcus kyotonensis Li et al., 2007; Rhodococcus maanshanensis Zhang, et al., 2002; Rhodococcus marinonascens Helmke and Weyland, 1984; Rhodococcus nanhaiensis; Rhodococcus olei Chaudhary and Kim, 2018; Rhodococcus opacus Klatte, et al., 1995; Rhodococcus percolatus Briglia, et al., 1996; Rhodococcus phenolicus Rehfuss and Urban, 2006; Rhodococcus polyvorum Li et al. 2012; Rhodococcus pyridinivorans Yoon, et al., 2000; Rhodococcus qingshengii Xu et al. 2007; Rhodococcus rhodochrous (Zopf 1891) Tsukamura, 1974; Rhodococcus rhodnii Goodfellow and Alderson, 1979 (synonym: Nocardia rhodnii); Rhodococcus ruber (Kruse 1896) Goodfellow and Alderson, 1977 (synonym: Streptothrix rubra Kruse, 1896); Rhodococcus jostii RHA1; Rhodococcus soli Li et al. 2015; Rhodococcus triatomae Yassin, 2005; Rhodococcus trifolii Kämpfer et al. 2013; Rhodococcus tukisamuensis Matsuyama, et al., 2003; Rhodococcus wratislaviensis (Goodfellow et al. 1995) Goodfellow, et al., 2002 (synonym: Tsukamurella wratislaviensis Goodfellow, et al., 1995); Rhodococcus yunnanensis Zhang, et al., 2005; or Rhodococcus zopfii Stoecker, et al., 1994

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to chemical and biological reagents for the valorization of PET. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet website specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice.

However, those of skill in the art should, in light of the present disclosure, appreciate that many changes can be made in the specific embodiments that are disclosed and still obtain a like or similar result without departing from the spirit and scope of the present disclosure.

### Example 1: Open-Loop Upcycling of Waste Poly(Ethylene Terephthalate) to Lycopene

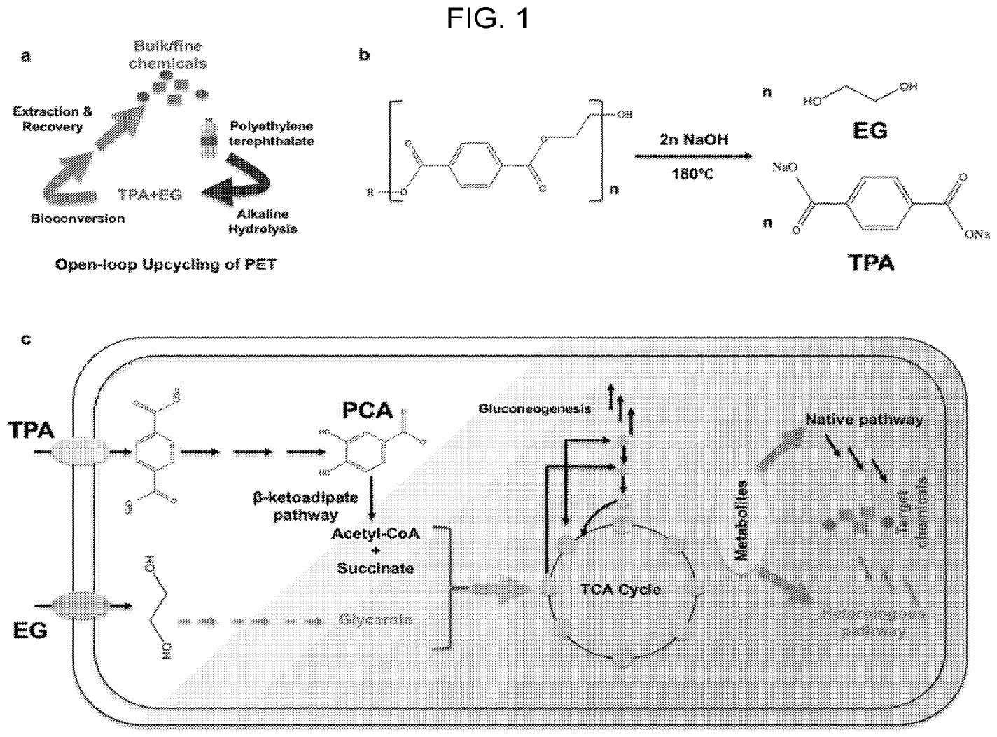

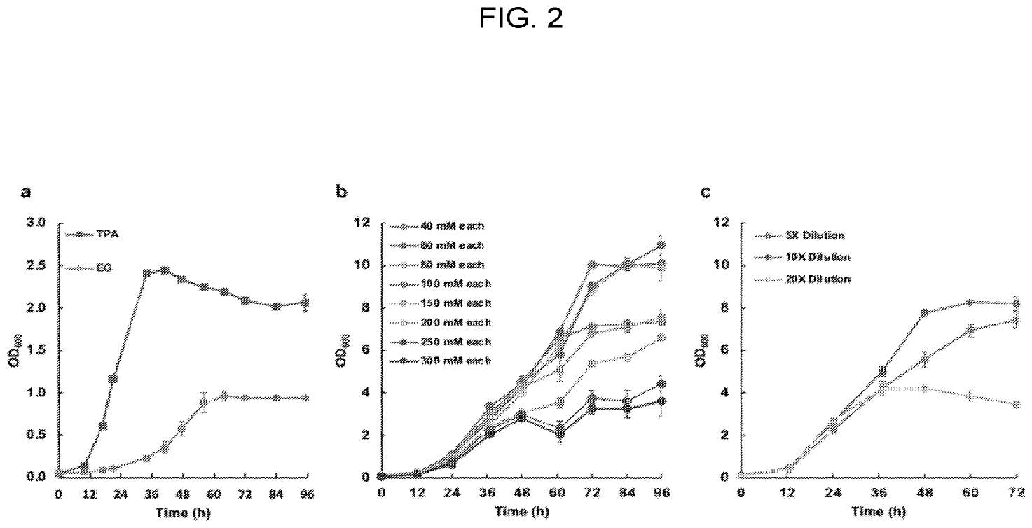

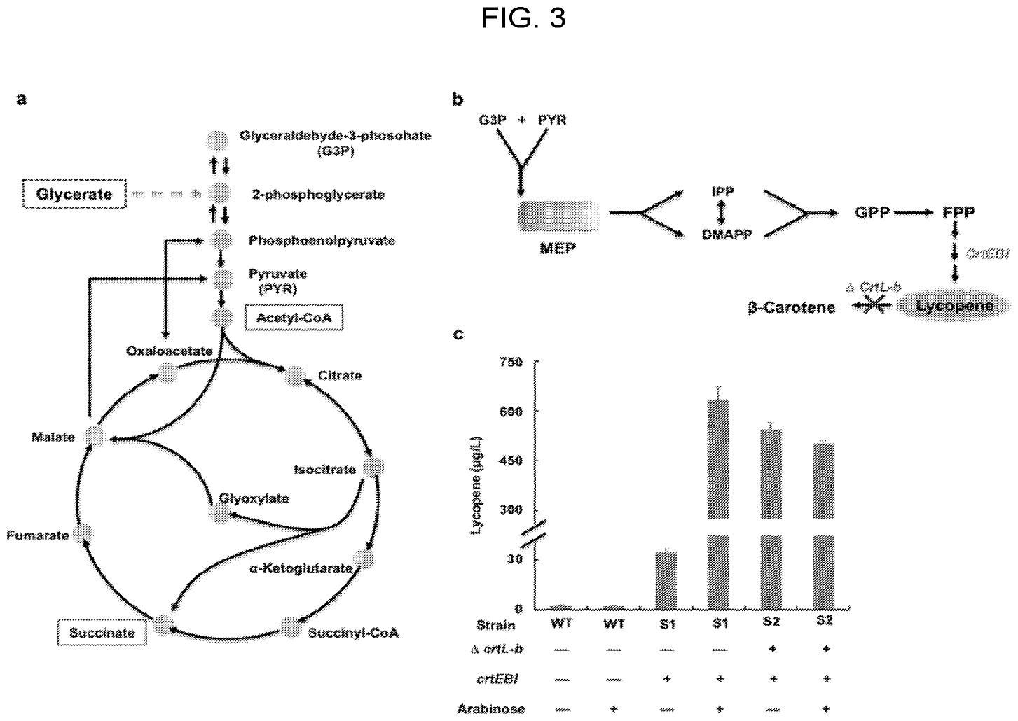

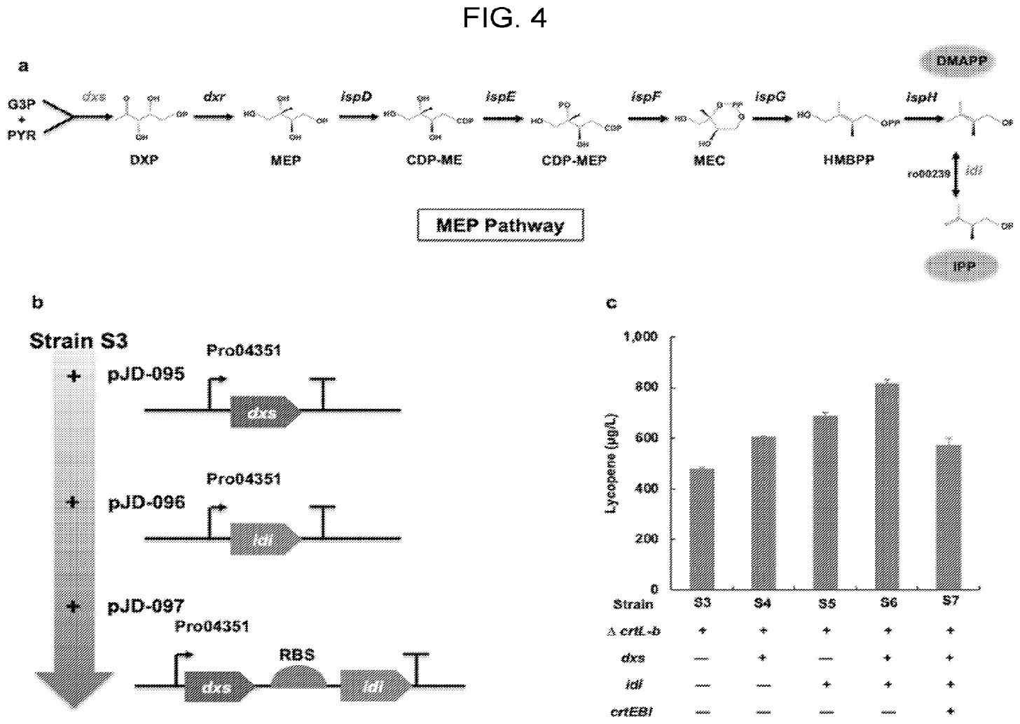

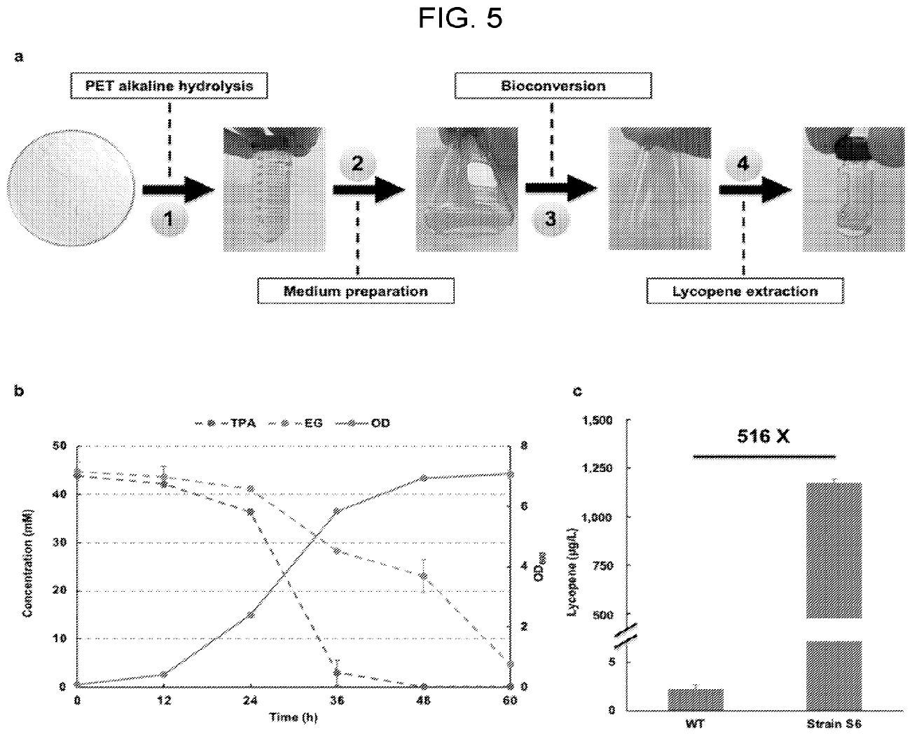

This example describes the open-loop upcycling of PET to value-added chemicals by an engineered RPET strain (see e.g., FIG. 1). The strains were screened and tested by synthetic mixture and PET hydrolysate (see e.g., FIG. 2). The RPET strain was engineered for the conversion of TPA and EG to lycopene (see e.g., FIG. 3). The isopenthenyl pyrophosphate (IPP) precursor supply was optimized for the production of lycopene (see e.g., FIG. 4). Bio-upcycling of PET to lycopene was demonstrated (see e.g., FIG. 5).

### Abstract

At present estimation, the most abundant polyester plastic poly(ethylene terephthalate) (PET) is manufactured at an annual volume of almost 70 million tons worldwide, with significant growth expected in the near future. The accumulation of waste PET in biosphere is now a global pollution crisis, and it is therefore urgent to cultivate technologies to valorize post-consumer PET and tackle the challenge of end-of-life management. The development of chemocatalytic and enzymatic approaches for depolymerizing PET to its corresponding monomers opens up new opportunities for PET upcycling through biological transformation. To this end, a new bacterial strain—Rhodococcus jostii strain PET (RPET)—with the ability to directly use PET hydrolysate as a feedstock was first identified. Then, the potential of RPET to upcycle PET into value-added chemicals, using lycopene as a proof-of-concept product was estimated. Through rational metabolic engineering, lycopene production was improved by more than a 500-fold over the wild-type. Finally, production of approximately 1200 μg/L lycopene from post-consumer PET via cascading this strain with the PET alkaline hydrolysis was demonstrated. In summary, this work highlights the great potential of biological conversion as a means to achieve post-consumer PET upcycling.

### INTRODUCTION

Man-made polymers are light-weight, sturdy and inexpensive, making them widely used in different industrial sectors and thus essential to modern society. It has been estimated that, worldwide, about 359 million tons of plastics are produced annually¹. Of this total, 150-200 million tons accumulate in landfills, where there is a high likelihood of being released into the natural environment and presenting what has been recognized as one of the most challenging ecological issues. Consequently, in order to combat the accumulation of waste plastics in the biosphere, there is an urgent demand to develop recycling technologies which enable a circular plastics economy.

Poly(ethylene terephthalate) (PET) is the most abundant polyester plastic manufactured in the world, with versatile applications including single-use packaging, clothing, and carpeting. The global annual production of PET exceeds 70 million tons, but only a minor fraction (<20%) of that volume is recycled². Traditionally, PET recycling is conducted via mechanical and/or chemical methods³. In mechanical recycling, the long polymer chains undergo extensive chain scission, a process which in most cases yields less than 10% of the input as usable output⁴. Due to its inefficient recycling rate and the inferior properties of the remanufactured materials, this process is often called ‘downcycling’^{5, 6}. Chemical reclamation—which deconstructs the polymer back to its monomeric components—holds the potential to address the efficiency challenge, and has been applied to various plastic waste streams, including high-density polyethylene (HDPE), polystyrene (PS), nylon-6, and PET⁷. In an industry context, the resulting monomers from the chemical deconstruction processes can be remanufactured into the same material or consumer good (e.g., bottle-to-bottle recycling) via closed-loop recycling. Given the high processing costs relative to the purchase of virgin PET, however, this recycling strategy has, to date, limited success. The feasibility of PET recycling could be increased by adding additional value to this waste (i.e., upcycling the resulting monomers to chemicals of higher value) via open-loop recycling. This concept, which begins with the chemical depolymerization of PET, has been demonstrated several times. In an early example, Rorrer and colleagues reported that PET could be upcycled to higher-value, longer-lifetime fiber-reinforced plastics (FRPs) via combination with biomass-derived monomers⁸. In another study, PET was upcycled to dioctyl terephthalate (DOTP) by alcoholysis with 2-ethyl-1-hexanol (2-EH) as the solvent and choline chloride-based deep eutectic solvents (ChCI-based DESs) as the catalysts⁹. More recently, photoreforming and electrocatalytic strategies have been developed to convert PET waste into clean H₂ fuel and commodity chemicals¹⁰⁻¹².

After decades of PET use, and subsequent leakage into the biosphere, a microbe has been isolated with enzymatic and catabolic adaptations permitting the degradation of PET as carbon and energy sources¹³, enabling the development of a new, biotechnological path for upcycling PET. In principle, after PET is depolymerized into its monomers terephthalic acid (TPA) and ethylene glycol (EG) via chemical hydrolysis or enzymatic degradation, this carbon-rich waste stream can then be used as feedstock for microbes to produce chemicals¹⁴. This process mirrors the well-known valorization of lignocellulose, wherein biomass is depolymerized into an array of aromatic monomers before being fed to microbes. However, unlike plant biomass, PET hydrolysate is less complex—consisting almost entirely of TPA and EG units—making it much more accessible for biotechnological valorization^{15, 16}. Similar to depolymerized lignin, TPA and EG monomers from enzymatically degraded PET have been used as feedstocks for the production of polyhydroxyalkanoate (PHA) and a novel bio-based poly(amide urethane) (bio-PU) by Pseudomonas umsongensis GO1616. However, the economic sustainability of this bioconversion for bio-plastics is still questionable. Additionally, through metabolic engineering, monomeric TPA obtained from PET depolymerization can be converted into intermediates of aromatic catabolismor example, an Escherichia coli chassis has been developed to directly upcycle PET-derived TPA into vanillin¹⁷. In another study, PET was deconstructed by chemocatalytic glycolysis, and the resulting bis(2-hydroxyethyl) terephthalate (BHET) was then catabolized by Pseudomonas putida KT2440—heterologously expressing PETase, mono(2-hydroxyethyl) terephthalic acid digesting enzyme (MHETase), TPA transporters, and TPA-to-PCA conversion enzymes—into β-ketoadipic acid via TPA¹⁸. However, in these two examples, an additional carbon source (e.g., glucose) was required to support cell growth, making both processes economically unsustainable.

In this study, we attempted to address these challenges by upcycling PET to value-added chemicals in a microbial system which uses PET hydrolysate as the sole carbon source. Combined with the highly-efficient alkaline hydrolysis, we developed a hybrid upcycling strategy for converting PET into lycopene—a potential platform intermediate with a wide variety of applications—by using alkaline hydrolysis of PET and the engineered microbial chassis Rhodococcus jostii strain PET (hereafter RPET) (FIG. 1). Specifically, PET was depolymerized into its monomers via alkaline hydrolysis, and the resulting hydrolysate was then used to support cell growth of RPET. We estimated the potential of RPET to upcycle PET into value-added chemicals by using lycopene as a sample product. With the integration of multidisciplinary metabolic engineering strategies, we accomplished a 500-fold increase in production over the WT strain, generating 1200 μg/L lycopene from PET hydrolysate alone. Overall, we present a novel bio-upcycling route for PET waste, which paves a new path towards a circular plastic economy.

### Results

### Screening Microbial Chassis Capable of Utilizing Both TPA and EG.

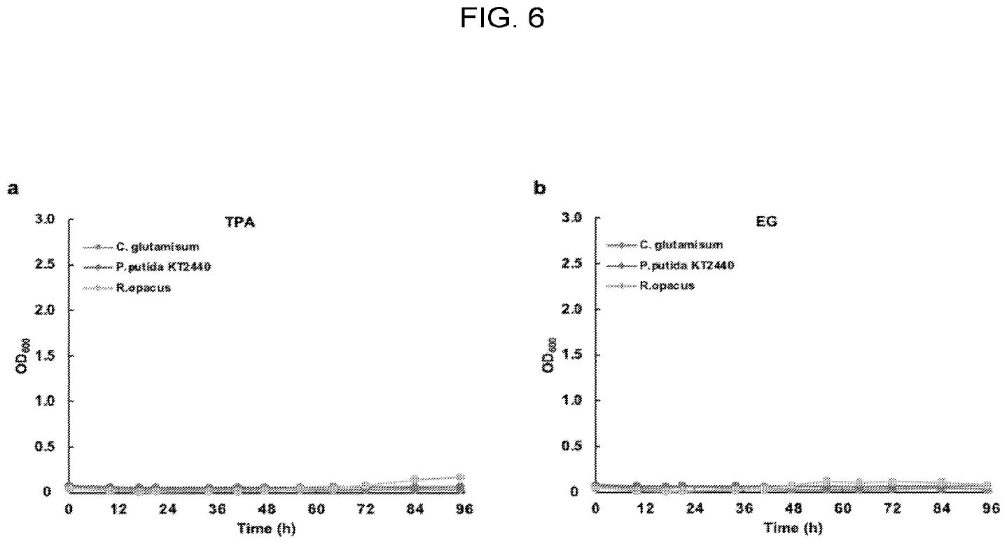

In a previous attempt at bio-upcycling PET, leaf-branch compost cutinase (LCC) was employed to enzymatically hydrolyze PET, a process which is relatively inefficient and time-consuming¹⁶. In contrast, alkaline hydrolysis of PET can achieve near-complete depolymerization of PET in one hour, with the reaction yield reaching up to 97.9%19. Despite the promisingly high efficacy of this method, the resulting hydrolysate always has high osmolarity, which presents a challenge for bio-upcycling via microbial culture. Additionally, the two primary components of PET hydrolysate—TPA and EG—require separate pathways to enter the cell's primary metabolic network and be converted into target chemicals. Therefore, the ideal microbial chassis would have both the potential to tolerant osmotic stress and the capacity to utilize both TPA and EG. To this end, we first screened several different strains of bacteria (Corynebacterium glutamicum, Pseudomonas putida KT2440, RPET, and Rhodococcus opacus PD630) for growth on both monomers, using 15 mM TPA or 15 mM EG as sole carbon sources. Cell growth on both compounds as the sole carbon sources was found only in RPET (FIG. 1 and FIG. 6). Surprisingly, only a two-day lag phase was observed in RPET when using EG as sole carbon source (FIG. 1a), which is much faster than the previously reported EG-degrading strain P. umsongensis GO1616.

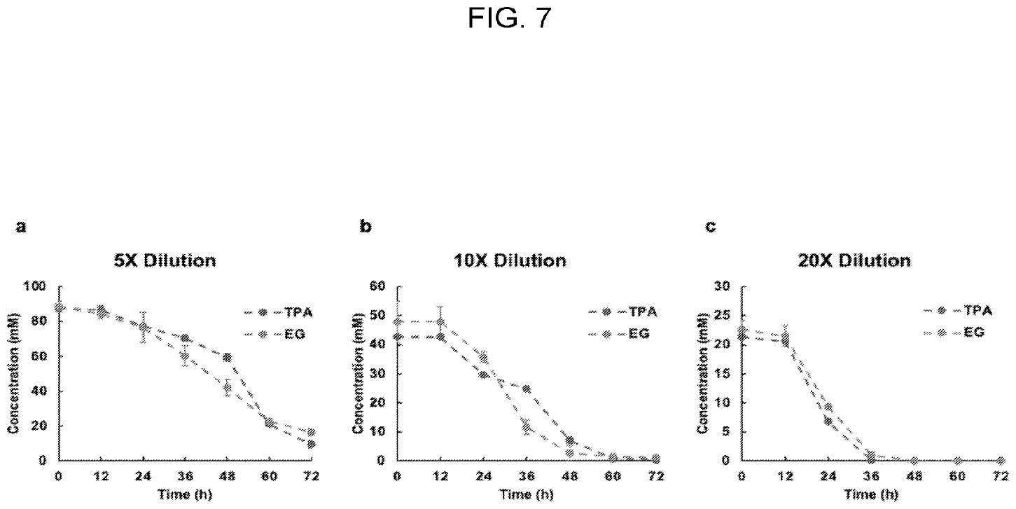

To test the feasibility of using RPET as a microbial chassis for the bio-upcycling of PET, we prepared equimolar mixtures of TPA disodium salt and EG—ranging from 40 mM to 300 mM each—to mimic the PET hydrolysate. When the concentrations of these simulated hydrolysates increased from 40 to 100 mM each (Na⁺ concentration 80 to 200 mM), RPET showed enhanced final cell density, with no significant differences observed in the duration of lag phase (FIG. 2b). In contrast, when exposed to higher concentrations (150 to 300 mM each), obvious inhibition of cell growth was observed (FIG. 2b), suggesting that the bacteria can tolerate no more than 200 mM Na⁺. To test if the PET hydrolysate could support similar cell growth to that observed with the simulated hydrolysates, RPET was grown in minimal medium supplied with PET hydrolysate as sole carbon source, at three dilution factors (5, 10, and 20-fold). RPET growth was uninhibited when exposed to 10- and 20-fold diluted PET hydrolysate (approximately 43 mM each and 21 mM each TPA and EG, respectively) (FIG. 2c), and HPLC quantification of each monomer in the spent media indicated complete consumption in both conditions (FIG. 7). However, in the 5-fold dilution condition (approximately 86 mM each of TPA and EG), the increased hydrolysate concentration had a negative effect on cell growth (FIG. 2c). This inhibition might result from the toxicity of the high Na⁺ concentration in the 5-fold dilution condition, which was approximately 400 mM and well above what was tolerated in the simulated hydrolysate experiments. Despite the osmotic limitations, these results indicated that RPET would be an ideal biological chassis to pair with alkaline hydrolysis for the biotechnological upcycling of PET.

### Genetic Engineering of RPET for the Biosynthesis of Lycopene.

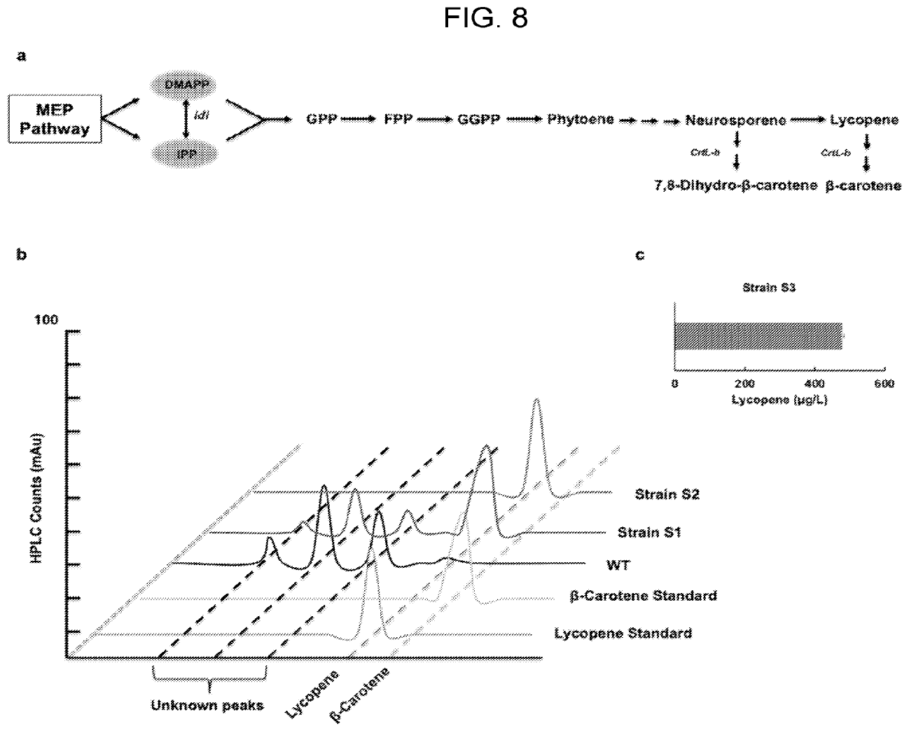

Rhodococcus sp. strains represent promising candidates for the production of compounds with medical and environmental relevance. Carotenoids are yellow to deep red pigments originated from the terpenoid biosynthetic pathway, and are commonly found in many Rhodococcus sp. strains with different types²⁰. Recently, the potential of Rhodococcus jostii RHA1 for carotenoid production has been explored, with 0.3 mg/L carotenoid measured after a 96 h of fermentation on lignin-based compounds²¹. To demonstrate upcycling of PET in RPET, we chose lycopene as the target product. This high-value carotenoid has antioxidative and anti-inflammatory activities which have been extensively studied in health applications, including the prevention of cancer and the reduction of cardiovascular and Alzheimer's disease risk factors²²⁻²⁴. Based on the genome sequence of related strains, we have putatively identified the enzymes of the carotenoid biosynthetic pathway in RPET (FIG. 3b and FIG. 8a). To test the feasibility of converting PET into lycopene, we began by feeding the WT strain with 20 mM TPA and 20 mM EG as carbon sources. Carotenoids extracted from stationary phase cells included lycopene at very low concentration (˜2 μg/L), as well as three unknown carotenoid compounds (FIG. 3c and FIG. 8b).

In Corynebacterium glutamicum, it has been reported that the overexpression of prenyltransferase—(CrtE), phytoene synthase—(CrtB), and phytoene desaturase—(CrtI) encoding genes significantly improved the flux from the precursor molecules isopentenyl pyrophosphate (IPP) and dimethylallyl pyrophosphate (DMAPP) to lycopene²⁵. To evaluate whether this strategy would work in RPET, we placed the synthetic operon crtEBI under the control of the arabinose-inducible promoter pBAD on a shuttle plasmid and transformed RPET strain, resulting in Strain S1. The lycopene assay revealed that in Strain S1, 630 μg/L lycopene was accumulated when the operon was fully-induced (50 mM arabinose), a 300-fold improvement over the WT (FIG. 3c). Notably, 34 μg/L lycopene was detected in Strain S1 in the absence of arabinose, suggesting leaky expression of the crtEBI operon, driven by the pBAD promoter (FIG. 3c). The previously-observed unknown carotenoid compounds, while still present in Strain S1 samples, occurred at notably decreased concentrations (FIG. 7b), suggesting that the biosynthesis of lycopene is mutually competitive with those compounds. A closer examination of the native carotenoid biosynthetic pathway revealed the putative lycopene beta cyclase (CrtL-b) can not only convert lycopene into β-carotene, but also catalyze neurosporene to produce 7,8-dihydro-β-carotene (FIG. 8a). The unknown compounds might therefore be synthesized via one or both these two competitive pathways. Consequently, we hypothesized that knocking out crtL-b would be beneficial for lycopene accumulation. To validate this hypothesis, we replaced the crtL-b gene in Strain S1 with a hygromycin-resistance selective marker, which resulted in the generation of Strain S2. Although carotenoid analysis revealed that the three unknown peaks had been eliminated in Strain S2, no changes in the lycopene production were observed, irrespective of arabinose treatment, indicating that this level of lycopene production was independent of the synthetic crtEBI operon (FIG. 3c and FIG. 8b). Inspired by a study in C. glutamicum, where knocking out the lycopene elongase enabled the accumulation of lycopene²⁶, we suspected that the production of lycopene in Strain S2 might be attributed to the deletion of crtL-b. As expected, when we knocked out crtL-b in the WT strain (generating the Strain S3 mutant), about 480 μg/L lycopene was produced (FIG. 8c), which supported our hypothesis that deletion of crtL-b contributed to lycopene accumulation in Strain S2. Additionally, when compared to Strain S3, overexpression of the synthetic crtEBI operon in Strain S2 failed to enhance lycopene production, indicating that the key factor in determining its lycopene production should be the supply of precursors.

### Optimization of the IPP Precursor Supply for Enhancing Lycopene Production.

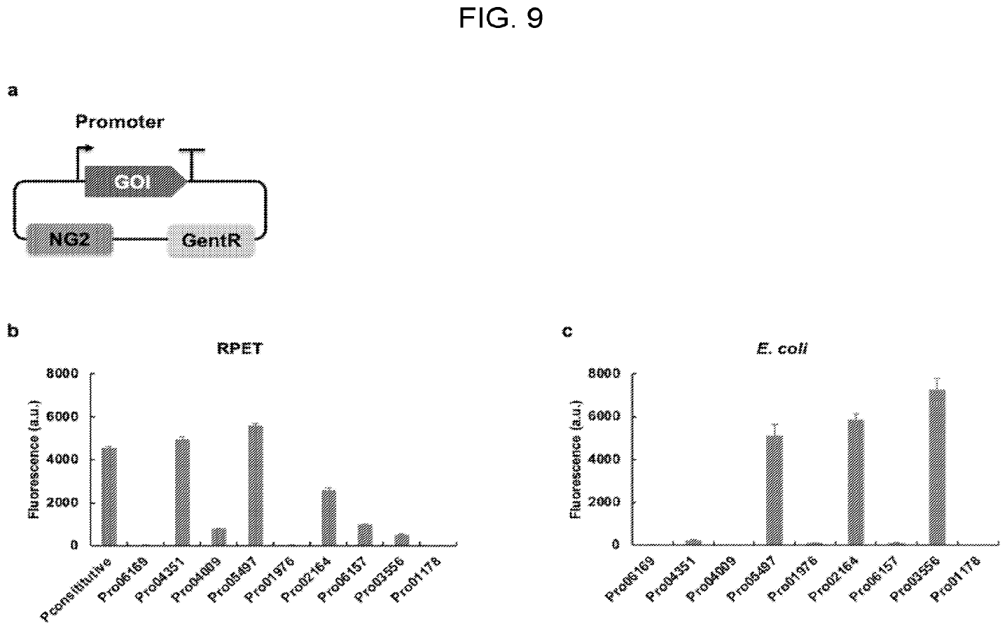

Carotenoids are synthesized from two common precursors—IPP and DMAPP—that are the end-products of the 2-methylerythritol 4-phosphate (MEP) pathway. In RPET, the native MEP pathway consists of nine reactions catalyzed by eight enzymes (FIG. 4a). The first reaction in the MEP pathway is the Dxs-catalyzed condensation of pyruvate and glyceraldehyde 3-phosphate to form DXP, which has been recognized as the key rate-limiting reaction in controlling carbon flux through the MEP pathway^{27, 28}. To test if Dxs is a bottleneck for carotenoid biosynthesis in RPET, we wanted to overexpress the dxs gene in Strain S3. The overexpression of genes of interest is typically performed by using a self-replicating plasmid that can be maintained at multiple copies per cell, which could have relatively high protein abundance versus genes integrated into the genome. To make the dxs-overexpression plasmid compatible with the plasmid used for expressing the synthetic crtEBI operon, we selected a backbone with the pNG2 origin of replication (FIG. 9a). Due to the limited selection of promoters validated in RPET, we first borrowed several characterized ones from related Actinomycetales, such as R. opacus PD63029. However, plasmid assembly always failed, which may be traced to the promoters' high activities in E. coli and the consequent metabolic burden of the constitutively-overexpressed dxs. To circumvent this shortcoming, we next sought to screen several promoters from the close strain R. jostii RHA1 which are of relatively high activities in RPET but non-functional in E. coli. A total of 9 predicated promoter sequences located upstream of different classes of proteins—including heat shock protein, cold shock protein, translation initiation factor, molecular chaperonin and RNA polymerase—were tested in both RPET and E. coli. In RPET, the measured expression level of Pro04351 was almost identical to a well-characterized, strong synthetic promoter from Streptomyces lividans TK24, but demonstrated low activity in E. coli to meet the criteria for use in this application (FIG. 9b and FIG. 9c). Using this new, E. coli-incompatible promoter, we assembled a plasmid to overexpress a C. glutamicum-sourced dxs gene and transformed Strain S3; the resulting Strain S4 demonstrated a 27% increase in lycopene production over its predecessor, reaching 606 μg/L (FIG. 4b and FIG. 4c).

In the MEP pathway, IspH synthesizes both IPP and DMAPP, but typically not in equimolar amounts: for example, in E. coli IPP is synthesized at a 5:1 proportion to DMAPP³⁰. In C. glutamicum, the unbalanced biosynthesis of IPP and DMAPP frequently impairs cell growth and carotenogenesis³¹. We therefore proposed that balancing the IPP and DMAPP metabolite pools could enhance the lycopene production. To test this hypothesis, C. glutamicum isopentenyl pyrophosphate isomerase gene (idi) was isolated and overexpressed in Strain S3, yielding Strain S5 (FIG. 4b). As expected, lycopene production increased by 44%, reaching 687 μg/L in Strain S5 (FIG. 4c). To test if the co-expression of idi with dxs could synergistically improve the lycopene production, a synthetic dxs-idi operon was expressed in Strain S3, resulting Strain S6 (FIG. 4b). Lycopene production in Strain S6 demonstrated a 71% improvement, reaching 817 μg/L (FIG. 4c). However, when the dxs-idi and CrtEBI operons were co-expressed in Strain S7, lycopene production reverted to Strain S4 levels (FIG. 4c).

### Bioconversion of PET Alkaline Hydrolysate to Lycopene.

Finally, to cement the proof-of-concept for the upcycling of PET to value-added chemicals, we pursued lycopene production from PET hydrolysate, rather than equimolar TPA and EG monomers (FIG. 5a). Semi-crystalline PET was purchased from Goodfellow (Huntingdon, England) for alkaline hydrolysis, as previously described¹⁹. The resulting crude, depolymerized product was centrifuged to remove the solid residues, and the clear supernatant was extracted for HPLC analysis to determine the concentrations of each monomer. HPLC detected 428.8±15.96 mM TPA and 433.8±37.21 mM EG, which represented a depolymerization rate between 82.3% (based on TPA content) and 83.3% (based on EG content) (TABLE 1). This is a substantial improvement over the enzymatic method, wherein unmodified leaf-branch compost cutinase (LCC; a polyester hydrolase) enabled only 47% depolymerization of amorphous PET films within 24 hs¹⁶. In another study, the novel LCC variant ICCG was able to demonstrate 90% depolymerization of PET within 9.3 hs², but when the depolymerization rate is considered in concert with the reaction time for, the natural conclusion is that alkaline hydrolysis is of higher efficiency. Moreover, the post-reaction broth from the alkaline hydrolysis reaction can be used as “drop-in” carbon source to feed the RPET strain without purification prior to use, which reduces costs while maintaining a high PET recycling ratio.

TABLE 1
Analysis of the PET hydrolysate.

| | | | | Hydrolysis |
|---|---|---|---|---|
| | | TPA | EG | efficiency |
| Depolymerization | PET | Concentration | Concentration | (Based on |
| method | (mM) | (mM) | (mM) | TPA) |
| Alkaline | 520.8 | 428.8 ± 15.96 | 433.8 ± 37.21 | 83% |
| hydrolysis | | | | |
| Enzymatic | 104.2 | 15.8 ± 0.34 | 18.0 ± 2.51 | 15% |
| hydrolysis | | | | |

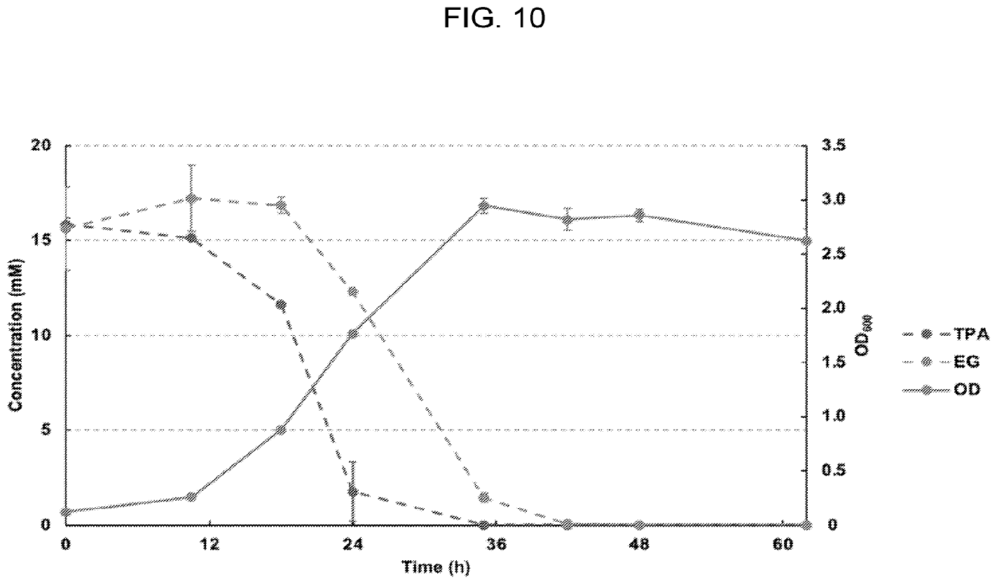

To demonstrate that the PET-derived monomers could be converted to lycopene, Strain S6 was inoculated into the minimal medium with 10% (v/V) crude PET hydrolysate as carbon source (initial concentration of each monomer ˜43 mM). TPA was completely consumed within 48 h of inoculation, while EG was consumed at a slower rate and did not reach complete depletion by the end of the fermentation period (FIG. 5b). Peak cell density was achieved after 48 h of cultivation, and remained steady for the next 12 h. A biomass yield of 0.366±0.005 g_{cell dry weight}/g_{substrate} was achieved, which is 75% higher than that of P. umsongensis GO16 strain when fermented with enzymatically-generated PET hydrosylate¹⁶. The final lycopene titer was approximately 1200 μg/L, which is similar to the production demonstrated by Strain S6 when cultivated in the simulated hydrolysate of 50 mM each TPA and EG (FIG. 10). These results demonstrate that conversion of PET to lycopene is achievable in RPET, with the implementation of synthetic biology strategies to improve yield.

### DISCUSSION

Global plastics pollution has recently been highlighted as a serious crisis, eliciting a strong need to develop new technologies for recycling plastics to realize a fully circular plastic economy. In this study, by cascading alkaline hydrolysis of PET with microbial conversion, we developed an open-loop upcycling strategy to recycle commercial PET into value-added chemicals. In this process, both monomers generated from alkaline-hydrolyzed PET can be directly employed as feedstocks to support cell growth of the microbial chassis, avoiding the need for monomer purification after hydrolysis and providing a simple route for microbial conversion. Additionally, in a first-of-its-kind demonstration to improve the economics of waste PET recycling, we experimentally validated the biological upcycling of PET into lycopene through rational genetic engineering. This proof-of-concept establishes the potential of this bio-upcycling strategy to be commercially profitable, and exemplifies the previously-proposed biotechnological solution to the problem of plastic pollution¹⁴.

Multiple approaches for depolymerizing PET have been established, including enzymatic hydrolysis, chemical hydrolysis, thermolysis, and chemical glycolysis^{18, 19}. Due to the heterogeneity of the deconstruction products generated by different depolymerization methods, the microbial chassis, metabolic engineering approach, and bioprocess must be selected rationally to optimize the bio-upcycling process. Alkaline hydrolysis of PET has been shown to be an efficient method for the regeneration of its constituent monomers, TPA and EG¹⁹. However, the hydrolysate obtained from alkaline hydrolysis is usually hyperosmotic, which presents difficulties when cascading into microbial bioconversion. To date, only the Gram-negative bacteria Pseudomonas—which have a high potential for degrading synthetic plastic—have been developed as platforms to consume both TPA and EG for biotechnological upcycling of PET, using both genetic engineering and adaptive laboratory evolution strategies^{16, 18}. The hyperosmotic condition of the hydrolysate (often 2 M sodium), however, limits the fitness of these strains for the direct conversion of the hydrolysis-generated monomers, as they are sensitive to osmotic stress³². In contrast, our tests of RPET showed no significant growth inhibition when the sodium concentration was increased to 0.2 M. Moreover, even at 0.6 M sodium, we observed that the cells continued to survive and grow (FIG. 2b), indicating that this strain is a robust choice for this biotechnological application. Notably, it has been reported that the PET hydrolysate obtained from enzymatic hydrolysis with LCC can be used directly as a feedstock for the engineered P. umsongensis GO16 strain to produce bio-plastics¹⁶. In this scenario, the maximum load of the PET hydrolysate for fermentation was approximately 30 mM of each TPA and EG, with time-course measurements of the consumption of both monomers revealing that EG did not reach complete depletion after 27 h of incubation¹⁶. In our study, incomplete consumption of the monomers was only observed when the maximum load of the PET hydrolysate was increased to approximately 85 mM each of TPA and EG, which may be attributable to the high concentration of Na⁺ (FIG. 7). In addition to alkaline hydrolysis, PET hydrolysate obtained from ICCG—a powerful LCC variant²—can also be used directly as feedstock to support RPET cell growth (FIG. 10). The compatibility of RPET with the products of multiple PET depolymerization methods contributes to its substantial potential with respect to PET bio-upcycling.

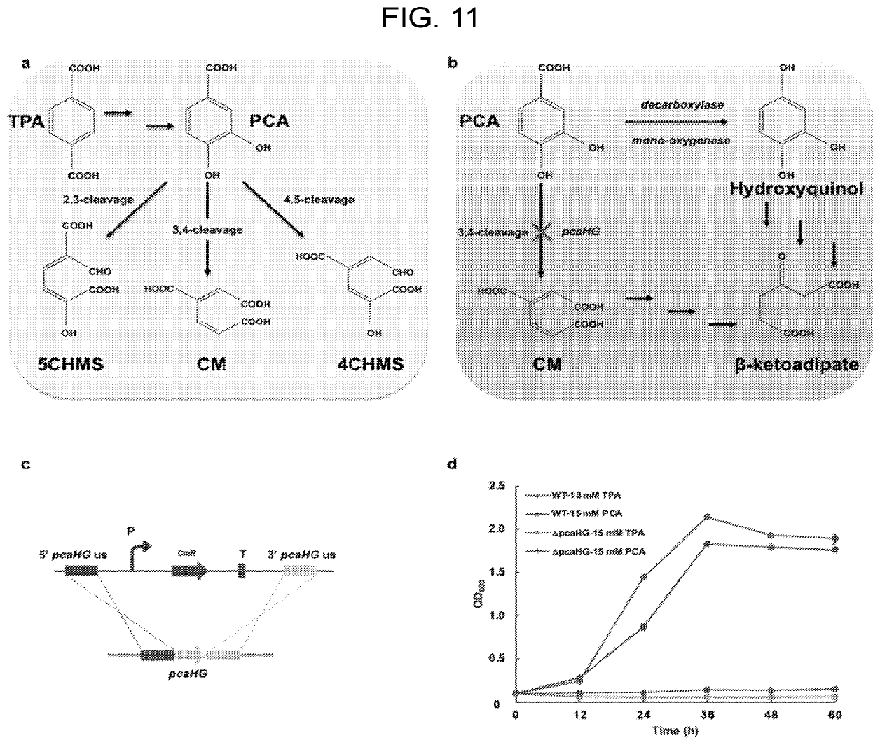

To date, native TPA catabolismhas been reported in several different strains, including Comamonas sp. E6, Ideonella sakaiensis, P. umsongensis GO16, and R. jostii RHA113, 33-35. The degradation of TPA begins with the conversion of TPA to protocatechuate (PCA), an important intermediate metabolite involved in the degradation of various aromatic compounds³⁶, which then feeds into one of three recognized catabolic pathways for PCA degradation (FIG. 11a): the PCA 2,3-cleavage pathway³⁷, the PCA 3,4-cleavage pathway³⁸, and the PCA 4,5-cleavage pathway³⁹. In R. jostii RHA1, PCA degradation is primarily managed by the PCA 3,4-cleavage pathway, but the hydroxyquinol degradation pathway has been identified as an alternative pathway which is induced only by high concentrations of PCA⁴⁰. Intriguingly, while RPET is a close relative of R. jostii RHA1, knocking out pcaHG (the operon which putatively encodes the PCA 3,4-cleavage pathway) abolished its ability to consume both TPA and PCA (FIG. 11b, FIG. 11c, and FIG. 11d), suggesting that this pathway is the sole catabolic pathway responsible for the degradation of TPA in RPET.

Various strains that can utilize EG as sole carbon and energy source have been described, both under obligate aerobic as well as anaerobic conditions⁴¹. More detailed studies have revealed that when the metabolic oxidation of EG occurs through the dicarboxylic acid pathway or the glyoxylate cycle, only reducing equivalents are produced, with no contribution to cell growth. In contrast, some strains (e.g., P. putida JM37) are able to generate biomass solely from EG, so the glyoxylate carboligase pathway and glyoxylate shunt have been proposed as an alternate catabolic pathway that allows biomass formation⁴². Although RPET can utilize EG as the sole carbon source to support cell growth, the full map of the EG catabolic pathway in this strain is still unclear due to incomplete genomic information. Additionally, no obvious catabolite repression between TPA and EG was observed in RPET, which is dramatically different from what has been observed in Pseudomonas strains^{16, 18}.

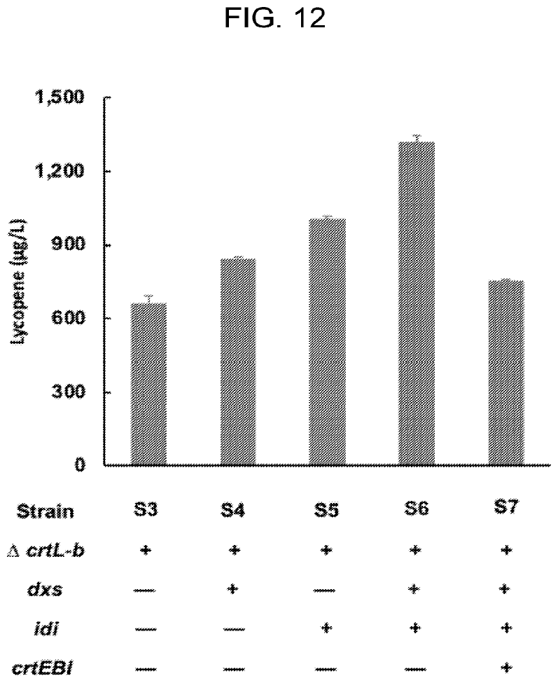

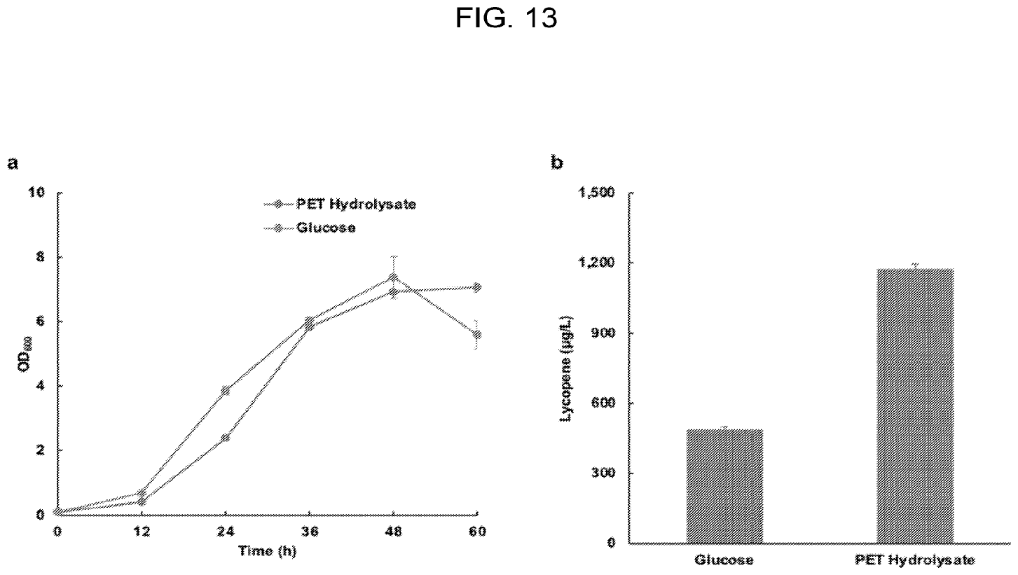

As more efficient PET depolymerization methods are described, there are commensurate efforts to develop strategies for recycling of the resultant monomers⁴³. Among these methods, bio-upcycling the TPA and EG monomers present in PET hydrolysate has great economic merits, as through a tremendously versatile array of metabolic pathways those monomers can be converted to products with higher value than PET itself. For example, recent studies have demonstrated the biological conversion of PET monomers into other plastics, such as polyhydroxyalkanoate (PHA)¹⁶ and β-ketoadipic acid¹⁸. The economic sustainability of bio-converting PET into bio-plastics is, however, still questionable. In this study, we sought to address this challenge by upcycling PET to the value-added chemical lycopene, using targeted genomic modifications to dramatically increase titer. In one such modification, we show that RPET overproduces lycopene when crtL-b is deleted (FIG. 3c and FIG. 8c). This ΔcrtL-b mutant (Strain S3 above) achieved a lycopene titer of 478 μg/L, which is a 236-fold improvement over the WT strain. It has been reported that, beyond making improvements to the terminal carotenoid biosynthetic pathway, modulating the supply of the precursors (e.g., IPP and DMAPP) is also important to carotenoid production⁴⁴. In a practical demonstration of this principle, it has been shown here that while the overexpression of the synthetic operon crtEBI did not improve lycopene titer in Strain S3, overexpressing genes for rate-limiting steps of the MEP pathway (i.e., dxs, idi) to enhance the supply of IPP and DMAPP resulted in incremental increases to lycopene titer (FIG. 3c and FIG. 4c). Although several studies have shown that the benefits of engineering individual aspects of the MEP pathway do not necessarily add up^{27, 45, 46}, our results demonstrated that the co-overexpression of dxs and idi has a synergistic effect on the lycopene production, achieving a titer 348-fold higher than that in the WT strain (FIG. 4c). Furthermore, it has been reported that in addition to fine-tuning key genes in the MEP pathway, overexpressing genes of the downstream pathway could improve the terpenoid production by metabolic pull³¹. In our case, however, co-expressing the synthetic dxs-idi and crtEBI operons in Strain S7 generated a significant decrease in lycopene production (FIG. 4c), suggesting that the combined metabolic burden from the high-level expression of several heterogenous proteins may compromise the lycopene production. Finally, using our best-production Strain S6, we successfully demonstrated the upcycling of PET to a value-added chemical. Our results showed that after 60 h of fermentation, the final lycopene production was approximately 1200 μg/L, which is similar to production fueled by synthetic, equimolar TPA and EG media (FIG. 12). As a comparison, we also conducted the fermentation by using 71.67 mM glucose (equal carbon content to the 10-fold diluted PET hydrolysate) as sole carbon source; though a higher growth rate was observed in the glucose condition, lycopene production was significantly decreased when compared to the PET condition (FIG. 13). These results further confirm that RPET is a robust and promising microbial workhorse for PET upcycling.

In summation, we report here a new biological upcycling method that, when paired with a high-efficient PET depolymerization method, enables the valorization of plastic waste towards the sustainable production of lycopene. By exploiting the metabolic versatility of RPET, many more valuable products (e.g., aromatics, organic acids, lipids and fuel molecules) could be sustainably produced through the upcycling of PET waste streams, contributing solutions to the challenges of the plastic pollution. There are, however, confounding challenges to address. First, post-consumer PET waste streams contain additives (e.g., co-monomers, dyes, and plasticizers)⁴⁷ which will accumulate as toxic compounds during the biological upcycling process. Identifying the toxic chemicals in these streams and expanding the catabolic pathways in RPET to compensate will be critical to increasing the carbon yield, which is of great significance to engineer a more efficient bioconversion chassis. Second, in contrast to lignocellulose, carbon-rich PET has only entered the biosphere in the last century, leaving little time for natural selection-driven enhancement of the hydrolytic enzymes and microbial consumers. Rational optimization of these enzymes, microbial chassis, and the accompanying technological processes is still needed to make the biotechnological upcycling of PET more commercially profitable.

### Methods

### Chemicals.

Disodium terephthalate was purchased from Alfa Aesar (Ward Hill, MA). poly(ethylene) terephthalate (PET) (max particle size 300 micron) was obtained from Goodfellow (Huntingdon, England). All the other chemicals used in this work were obtained from either Sigma Aldrich (St. Louis, MO) or Merck (Darmstadt, Germany). All solvents used for analytical methods were of analytical grade.

### Strains and Cultivation.

Rhodococcus opacus PD630 (DSMZ 44193) was obtained from Deutsche Sammlung von Mikroorganismen und Zellkulturen GmnH. Pseudomonas putida KT2440 (ATCC® 47054) and Rhodococcus jostii strain PET (RPET) were kindly provided by Dr. Laura R. Jarboe from Iowa State University and Dr. Yingjie Tang from Washington University in St. Louis, respectively. All the wild type (WT) and the derived genetic engineered strains used in this study were cultivated at 30° C. with shaking at 250 rpm in tryptic soy broth (TSB) or the previously described minimal salts medium B⁴⁸ supplemented with appropriate carbon sources, as indicated specifically for each experiment. 200 mM TPA stocks were made in minimal salts medium B, and the pH value was gradually adjusted to 7.0 with NaOH. Stocks were sterilized before use via filtration.

### Plasmid Construction.

Genomic DNA of RPET was extracted by using a Promega Wizard™ Genomic DNA Purification Kit (Promega, USA). Routine PCR amplifications were conducted using Phusion High-Fidelity DNA Polymerase (NEB, USA), and all primers were synthesized by Integrated DNA Technologies (IDT, USA). PCR products were extracted from electrophoresis gels using a Zymoclean Gel DNA Recovery Kit (Zymo, USA). Plasmids were assembled via Gibson Assembly⁴⁹ and directly transformed into Escherichia coli DH10B for plasmid maintenance. All the plasmid inserts were confirmed by Sanger sequencing performed by GENEWIZ (South Plainfield, NJ). Specific details of the plasmids used in this study are summarized in TABLE 2.

TABLE 2
Plasmids used in this study.

| SEQ | | | | | | |
|---|---|---|---|---|---|---|
| ID | Plasmid | | Origin of | Antibiotic | Length | |
| NO: | name | Functional insert | Replication | Resistance | (bp) | Source |
| 1 | pDD57 | pConstitutive.GFP+ | PAL5000 | Kanamycin | 4776 | Lab |
| | | | | | | inventory |
| 2 | pGZ001 | pRO06169. GFP+ | PAL5000 | Kanamycin | 4913 | This |
| | | | | | | study, |
| | | | | | | derived |
| | | | | | | from |
| | | | | | | pDD57 |
| 3 | pGZ002 | pRO04351. GFP+ | pAL5000 | Kanamycin | 5060 | This |
| | | | | | | study, |
| | | | | | | derived |
| | | | | | | from |
| | | | | | | pDD57 |
| 4 | pGZ003 | pRO04009. GFP+ | pAL5000 | Kanamycin | 4921 | This |
| | | | | | | study, |
| | | | | | | derived |
| | | | | | | from |
| | | | | | | pDD57 |
| 5 | pGZ004 | pRO05497. GFP+ | pAL5000 | Kanamycin | 4963 | This |
| | | | | | | study, |
| | | | | | | derived |
| | | | | | | from |
| | | | | | | pDD57 |
| 6 | pGZ005 | pRO01976. GFP+ | pAL5000 | Kanamycin | 5123 | This |
| | | | | | | study, |
| | | | | | | derived |
| | | | | | | from |
| | | | | | | pDD57 |
| 7 | pGZ006 | pRO02164. GFP+ | pAL5000 | Kanamycin | 4913 | This |
| | | | | | | study, |
| | | | | | | derived |
| | | | | | | from |
| | | | | | | pDD57 |
| 8 | pGZ007 | pRO06157. GFP+ | PAL5000 | Kanamycin | 4926 | This |
| | | | | | | study, |
| | | | | | | derived |
| | | | | | | from |
| | | | | | | pDD57 |
| 9 | pGZ008 | pRO03556. GFP+ | pAL5000 | Kanamycin | 4913 | This |
| | | | | | | study, |
| | | | | | | derived |
| | | | | | | from |
| | | | | | | pDD57 |
| 10 | pGZ009 | pRO01178. GFP+ | PAL5000 | Kanamycin | 4913 | This |
| | | | | | | study, |
| | | | | | | derived |
| | | | | | | from |
| | | | | | | pDD57 |
| 11 | pYH038 | A selection marker | p15A | Hygromycin | 4026 | This |
| | | in the center of an | (Integrative) | | | study, |
| | | ~2.0 kb genomic | | | | derived |
| | | fragment | | | | from |
| | | encompassing the | | | | pAGR318 |
| | | target gene CrtL-b | | | | |
| 12 | pJD078 | pBAD.CrtEBI | pAL5000 | Kanamycin | 8569 | This |
| | | | | | | study, |
| | | | | | | derived |
| | | | | | | from |
| | | | | | | pDD56 |
| 13 | pJD095 | pRO04351.dxs | NG2 | Gentamycin | 6488 | This |
| | | | | | | study, |
| | | | | | | derived |
| | | | | | | from |
| | | | | | | pDD67 |
| 14 | pJD096 | pRO04351.idi | NG2 | Gentamycin | 5165 | This |
| | | | | | | study, |
| | | | | | | derived |
| | | | | | | from |
| | | | | | | pDD67 |
| 15 | pJD097 | pRO04351.dxs.idi | NG2 | Gentamycin | 7105 | This |
| | | | | | | study, |
| | | | | | | derived |
| | | | | | | from |
| | | | | | | pDD67 |

Sequences.

pDD57 (SEQ ID NO: 1)
actagttgattagctaagcagaaggccatcctgacggatggcctttttgcgtttaatactgtttaaactctagaaatattggatc
gtcggcaccgtcacggccgtgggaggcggcacgatccgcgacgtgatgatcggccgcatccccacggtgctgcgcagtgag
ctctacgccatcccggcgttgatctgtgcgttcgctgtgcgggctctaacacgtcctagtatggtaggatgagcaacatttcga
cgccgagagattcgccgcccgaaatgagcacgatccgcatgcttaattaagaaggagatatacatatggctagcaaaggag
aagaacttttcactggagttgtcccaattcttgttgaattagatggtgatgttaatgggcacaaattttctgtcagtggagaggg
tgaaggtgatgctacatacggaaagcttacccttaaatttatttgcactactggaaaactacctgttccatggccaacacttgt
cactactttgacctatggtgttcaatgcttttcccgttatccggatcatatgaaacggcatgactttttcaagagtgccatgccc
gaaggttatgtacaggaacgcactatatctttcaaagatgacgggaactacaagacgcgtgctgaagtcaagtttgaaggtga
tacccttgttaatcgtatcgagttaaaaggtattgattttaaagaagatggaaacattctcggacacaaactcgagtacaact
ataactcacacaatgtatacatcacggcagacaaacaaaagaatggaatcaaagctaacttcaaaattcgccacaacattg
aagatggctccgttcaactagcagaccattatcaacaaaatactccaattggcgatggccctgtccttttaccagacaaccatt
acctgtcgacacaatctgccctttcgaaagatcccaacgaaaagcgtgaccacatggtccttcttgagtttgtaactgctgctg
ggattacacatggcatggatgagctctacaaataatgaattcgagctggatccagcttcctcgctcactgactcgctgcgctcg
gtcgttcggctgcggcgagcggtatcagctcactcaaaggcggtaatacggttatccacagaatcaggggataacgcagga
aagaacatgtgagcaaaaggccagcaaaaggccaggaaccgtaaaaaggccgcgttgctggcgtttttccataggctccgc
ccccctgacgagcatcacaaaaatcgacgctcaagtcagaggtggcgaaacccgacaggactataaagataccaggcgtttc
cccctggaagctccctcgtgcgctctcctgttccgaccctgccgcttaccggatacctgtccgcctttctcccttcgggaagcgt
ggcgctttctcaatgctcacgctgtaggtatctcagttcggtgtaggtcgttcgctccaagctgggctgtgtgcacgaacccccc
gttcagcccgaccgctgcgccttatccggtaactatcgtcttgagtccaacccggtaagacacgacttatcgccactggcagc
agccactggtaacaggattagcagagcgaggtatgtaggcggtgctacagagttcttgaagtggtggcctaactacggctac
actagaaggacagtatttggtatctgcgctctgctgaagccagttaccttcggaaaaagagttggtagctcttgatccggcaa
acaaaccaccgctggtagcggtggtttttttgtttgcaagcagcagattacgcgcagaaaaaaaggatctcaagaagatcctt
tgatcttttctacggggtctgacgctcagtggaacgaaaactcacgttaagggattttggtcatgagattatcaaaaaggatct
tcacctagatccttttggggtgggcgaagaactccagcatgagatccccgcgctggaggatcatccagccggcgtcccggaa
aacgattccgaagcccaacctttcatagaaggcggcggtggaatcgaaatctcgtgatggcaggttgggcgtcgcttggtcg
gtcatttcgaaccccagagtcccgctcagaagaactcgtcaagaaggcgatagaaggcgatgcgctgcgaatcgggagcgg
cgataccgtaaagcacgaggaagcggtcagcccattcgccgccaagctcttcagcaatatcacgggtagccaacgctatgtc
ctgatagcggtccgccacacccagccggccacagtcgatgaatccagaaaagcggccattttccaccatgatattcggcaag
caggcatcgccatgggtcacgacgagatcctcgccgtcgggcatccgcgccttgagcctggcgaacagttcggctggcgcga
gcccctgatgctcttcgtccagatcatcctgatcgacaagaccggcttccatccgagtacgtgctcgctcgatgcgatgtttcgc
ttggtggtcgaatgggcaggtagccggatcaagcgtatgcagccgccgcattgcatcagccatgatggatactttctcggcag
gagcaaggtgagatgacaggagatcctgccccggcacttcgcccaatagcagccagtcccttcccgcttcagtgacaacgtc
gagcacagctgcgcaaggaacgcccgtcgtggccagccacgatagccgcgctgcctcgtcttggagttcattcagggcaccg
gacaggtcggtcttgacaaaaagaaccgggcgcccctgcgctgacagccggaacacggcggcatcagagcagccgattgtc
tgttgtgcccagtcatagccgaatagcctctccacccaagcggccggagaacctgcgtgcaatccatcttgttcaatcatgcg
aaacgatcctcatcctgtctcttgatcagatcttgatcccctgcgccatcagatccttggcggcaagaaagccatccagtttact
ttgcagggcttcccaaccttaccagagggcgccccagctggcaattccggttcgcttgctgtccataaaaccgcccagtctagct
atcgccatgtaagcccactgcaagctacctgctttctctttgcgcttgcgttttcccttgtccagatagcccagtagctgacatt
catccggggtcagcaccgtttctgcggactggctttctacgtgttccgcttcctttagcagcccttgcgccctgagtgcttgcgg
cagcgtgaagctagcgtctaccaggacttttacctgtccgacccgttgcaacggaaccccccacggaacccccgcgacacccg
ctccccaattgcgttagaacagcggtggattgtcggcttcgttgtgggccttttgagccgcttcctgttctgccgcacgctcttt
cctcgcccgatagccgagtcgcttaacggtgtccagatgcagcccgaaatgtttggccgtttgcggccaagagtggccctcgtc
gtcgtgataggcgcggatgcgttcgcggcgtgcagcctgctcggcgagccactcgctgcgttcctgcgccacgagccggacg
acgtggcgttcggatagtccggtgattcgagcgccttcggcggcggtcacgcgccgctttttgcggacagtcggctgccggttg
tagccgtcgctgtagccgtcgctcatagcaatgcctccatggctgacgcggactttgcgcgccgcgcaactgtgctcgccgcc
gtgcgcgctgctgcgcccttccgcgagatggccgactggcgcgcactgagtgtggcctcgtagaccacgatcccgtccgccca
aatgcgcgacttggttgtgatccaacgccaaatgctgttggcgatggcgcggacctcgctgtccggtagcggtccgggacaca
cgtcgttgcacgggaactcggcgtttcgcgcgtggcactcggcatagatcgcgcggccgagtccgtccacgttccgggtcggc
aggtagatccgcatgagggcgggacgataggcccacaacctgacggaatcgaacagtgcgcaattccgccctagcggcgtc
ggagccgctttgtacgtggtctgctgacgccagcgcggcggtggcatgttcgcgccgagctcggcctcgatgtggctgagtgt
gtagagatctgagtggagccattccgtttcccaggcgatgtggccggggtttttggtcatgaggcctgagtaactgcggtcgcc
gtccacggcgcgccgaaggccttcggcgcacgccgccatgtatgcgagcggcttacgccgcgcgtattcggtgcgtggaaca
ggggcgttgagtgcccacactgcgtgtgcgtggccgttggcgcgattgcccacgatcgcgttgggcagcggatgggaccccc
gggcgctgagcgctcggagcgctgcgtctggatggtctacgtccacgaccagcaggtttgccagcgctgttgggttcgcctcg
atgtaccggcggcctagggccgacgcgcggctttggcggtagatcccctcgagcagatcgtcgcttgccagcggccagtacg
gcagccagagctgctcaaattcgtcggcgacgtggctcacgcttggtagtagaccacgattaatcaccggtgtatggtccgac
acgagctccaagtcagatatttcgctgaggggccaccccacaactgcacactcccccgctctcccgtcgagccctggtggtgg
aacaccagcgacagccgagcacccccaaccacctgtaccaacc
pGZ001 (SEQ ID NO: 2)
actagttgattagctaagcagaaggccatcctgacggatggcctttttgcgtttaatactgtttaaactctagaaatattggatc
gtcggcaccgtcacggccgtgggaggcggcacgatccgcgacgtgatgatcggccgcatccccacggtgctgcgcagtgag
ctctacgccatcccggcgttgatctgtgcgttcgctgagtggtgtgacttccgactcacgtgcggaggccgtgcgtacggagtg
cgaaaccgtcatcagggcgcgaggtgggcgcttccgaatctgactccgctggtcgggcccgtacggccgggttgcgcgcgaa
tcgctgcgaatccgggcgaataccgttcggtccgcaccgttactcttggagtgttccgctgagagcgggagcacacccatcgg
gggaggtgccactgtaacgccaggatattcagcaacgatggctagcaaaggagaagaacttttcactggagttgtcccaatt
cttgttgaattagatggtgatgttaatgggcacaaattttctgtcagtggagagggtgaaggtgatgctacatacggaaagctt
acccttaaatttatttgcactactggaaaactacctgttccatggccaacacttgtcactactttgacctatggtgttcaatgct
tttcccgttatccggatcatatgaaacggcatgactttttcaagagtgccatgcccgaaggttatgtacaggaacgcactatatc
tttcaaagatgacgggaactacaagacgcgtgctgaagtcaagtttgaaggtgatacccttgttaatcgtatcgagttaaaag
gtattgattttaaagaagatggaaacattctcggacacaaactcgagtacaactataactcacacaatgtatacatcacggca
gacaaacaaaagaatggaatcaaagctaacttcaaaattcgccacaacattgaagatggctccgttcaactagcagaccatt
atcaacaaaatactccaattggcgatggccctgtccttttaccagacaaccattacctgtcgacacaatctgccctttcgaaag
atcccaacgaaaagcgtgaccacatggtccttcttgagtttgtaactgctgctgggattacacatggcatggatgagctctaca
aataatgaattcgagctggatccagcttcctcgctcactgactcgctgcgctcggtcgttcggctgcggcgagcggtatcagct
cactcaaaggcggtaatacggttatccacagaatcaggggataacgcaggaaagaacatgtgagcaaaaggccagcaaaa
ggccaggaaccgtaaaaaggccgcgttgctggcgtttttccataggctccgcccccctgacgagcatcacaaaaatcgacgc
tcaagtcagaggtggcgaaacccgacaggactataaagataccaggcgtttccccctggaagctccctcgtgcgctctcctgt
tccgaccctgccgcttaccggatacctgtccgcctttctcccttcgggaagcgtggcgctttctcaatgctcacgctgtaggtat
ctcagttcggtgtaggtcgttcgctccaagctgggctgtgtgcacgaaccccccgttcagcccgaccgctgcgccttatccggt
aactatcgtcttgagtccaacccggtaagacacgacttatcgccactggcagcagccactggtaacaggattagcagagcga
ggtatgtaggcggtgctacagagttcttgaagtggtggcctaactacggctacactagaaggacagtatttggtatctgcgctc
tgctgaagccagttaccttcggaaaaagagttggtagctcttgatccggcaaacaaaccaccgctggtagcggtggtttttttg
tttgcaagcagcagattacgcgcagaaaaaaaggatctcaagaagatcctttgatcttttctacggggtctgacgctcagtgg
aacgaaaactcacgttaagggattttggtcatgagattatcaaaaaggatcttcacctagatccttttggggtgggcgaagaa
ctccagcatgagatccccgcgctggaggatcatccagccggcgtcccggaaaacgattccgaagcccaacctttcatagaag
gcggcggtggaatcgaaatctcgtgatggcaggttgggcgtcgcttggtcggtcatttcgaaccccagagtcccgctcagaag
aactcgtcaagaaggcgatagaaggcgatgcgctgcgaatcgggagcggcgataccgtaaagcacgaggaagcggtcagc
ccattcgccgccaagctcttcagcaatatcacgggtagccaacgctatgtcctgatagcggtccgccacacccagccggccac
agtcgatgaatccagaaaagcggccattttccaccatgatattcggcaagcaggcatcgccatgggtcacgacgagatcctc
gccgtcgggcatccgcgccttgagcctggcgaacagttcggctggcgcgagcccctgatgctcttcgtccagatcatcctgatc
gacaagaccggcttccatccgagtacgtgctcgctcgatgcgatgtttcgcttggtggtcgaatgggcaggtagccggatcaa
gcgtatgcagccgccgcattgcatcagccatgatggatactttctcggcaggagcaaggtgagatgacaggagatcctgccc
cggcacttcgcccaatagcagccagtcccttcccgcttcagtgacaacgtcgagcacagctgcgcaaggaacgcccgtcgtg
gccagccacgatagccgcgctgcctcgtcttggagttcattcagggcaccggacaggtcggtcttgacaaaaagaaccgggc
gcccctgcgctgacagccggaacacggcggcatcagagcagccgattgtctgttgtgcccagtcatagccgaatagcctctcc
acccaagcggccggagaacctgcgtgcaatccatcttgttcaatcatgcgaaacgatcctcatcctgtctcttgatcagatctt
gatcccctgcgccatcagatccttggcggcaagaaagccatccagtttactttgcagggcttcccaaccttaccagagggcgc
cccagctggcaattccggttcgcttgctgtccataaaaccgcccagtctagctatcgccatgtaagcccactgcaagctacctgc
tttctctttgcgcttgcgttttcccttgtccagatagcccagtagctgacattcatccggggtcagcaccgtttctgcggactgg
ctttctacgtgttccgcttcctttagcagcccttgcgccctgagtgcttgcggcagcgtgaagctagcgtctaccaggactttta
cctgtccgacccgttgcaacggaaccccccacggaacccccgcgacacccgctccccaattgcgttagaacagcggtggattg
tcggcttcgttgtgggccttttgagccgcttcctgttctgccgcacgctctttcctcgcccgatagccgagtcgcttaacggtgt
ccagatgcagcccgaaatgtttggccgtttgcggccaagagtggccctcgtcgtcgtgataggcgcggatgcgttcgcggcgtg
cagcctgctcggcgagccactcgctgcgttcctgcgccacgagccggacgacgtggcgttcggatagtccggtgattcgagcg
ccttcggcggcggtcacgcgccgctttttgcggacagtcggctgccggttgtagccgtcgctgtagccgtcgctcatagcaatg
cctccatggctgacgcggactttgcgcgccgcgcaactgtgctcgccgccgtgcgcgctgctgcgcccttccgcgagatggcc
gactggcgcgcactgagtgtggcctcgtagaccacgatcccgtccgcccaaatgcgcgacttggttgtgatccaacgccaaat
gctgttggcgatggcgcggacctcgctgtccggtagcggtccgggacacacgtcgttgcacgggaactcggcgtttcgcgcgt
ggcactcggcatagatcgcgcggccgagtccgtccacgttccgggtcggcaggtagatccgcatgagggcgggacgatagg
cccacaacctgacggaatcgaacagtgcgcaattccgccctagcggcgtcggagccgctttgtacgtggtctgctgacgcca
gcgcggcggtggcatgttcgcgccgagctcggcctcgatgtggctgagtgtgtagagatctgagtggagccattccgtttccc
aggcgatgtggccggggtttttggtcatgaggcctgagtaactgcggtcgccgtccacggcgcgccgaaggccttcggcgca
cgccgccatgtatgcgagcggcttacgccgcgcgtattcggtgcgtggaacaggggcgttgagtgcccacactgcgtgtgcgt
ggccgttggcgcgattgcccacgatcgcgttgggcagcggatgggacccccgggcgctgagcgctcggagcgctgcgtctgg
atggtctacgtccacgaccagcaggtttgccagcgctgttgggttcgcctcgatgtaccggcggcctagggccgacgcgcggc
tttggcggtagatcccctcgagcagatcgtcgcttgccagcggccagtacggcagccagagctgctcaaattcgtcggcgacg
tggctcacgcttggtagtagaccacgattaatcaccggtgtatggtccgacacgagctccaagtcagatatttcgctgagggg
ccaccccacaactgcacactcccccgctctcccgtcgagccctggtggtggaacaccagcgacagccgagcacccccaacca
cctgtaccaacc
pGZ002 (SEQ ID NO: 3)
actagttgattagctaagcagaaggccatcctgacggatggcctttttgcgtttaatactgtttaaactctagaaatattggatc
gtcggcaccgtcacggccgtgggaggcggcacgatccgcgacgtgatgatcggccgcatccccacggtgctgcgcagtgag
ctctacgccatcccggcgttgatctgtgcgttcgcttgatgtctccgggctgtcgtagggtacgggcgagtagcttccacaccgc
cacgacctgcgcatttgatgataacgagtccttttccggggctgtcgcccggggtgtcgtgacccccgcgccaatccgctgtttc
gtgcctgttcgctacaagatcaactttttgtgtgaattgttcggatttcgactgttgcgttcgcggagttcatggttgactacaa
ctggtcgcagcttctgtgttcgtaggcttgaaagagtgaacgttcgcaggatcgatgttgcgaacgcggtgctctgcttttcctg
agggggaaatctagaagtacagcggtcggaaccggcccggcggactgtatgtgctgtccgccgaatccggtggttagaaag
agaaggaaaagcatggctagcaaaggagaagaacttttcactggagttgtcccaattcttgttgaattagatggtgatgttaa
tgggcacaaattttctgtcagtggagagggtgaaggtgatgctacatacggaaagcttacccttaaatttatttgcactactgg
aaaactacctgttccatggccaacacttgtcactactttgacctatggtgttcaatgcttttcccgttatccggatcatatgaaa
cggcatgactttttcaagagtgccatgcccgaaggttatgtacaggaacgcactatatctttcaaagatgacgggaactacaag
acgcgtgctgaagtcaagtttgaaggtgatacccttgttaatcgtatcgagttaaaaggtattgattttaaagaagatggaaa
cattctcggacacaaactcgagtacaactataactcacacaatgtatacatcacggcagacaaacaaaagaatggaatcaa
agctaacttcaaaattcgccacaacattgaagatggctccgttcaactagcagaccattatcaacaaaatactccaattggcg
atggccctgtccttttaccagacaaccattacctgtcgacacaatctgccctttcgaaagatcccaacgaaaagcgtgaccac
atggtccttcttgagtttgtaactgctgctgggattacacatggcatggatgagctctacaaataatgaattcgagctggatcc
agcttcctcgctcactgactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaaccgtaaaaaggc
cgcgttgctggcgtttttccataggctccgcccccctgacgagcatcacaaaaatcgacgctcaagtcagaggtggcgaaacc
cgacaggactataaagataccaggcgtttccccctggaagctccctcgtgcgctctcctgttccgaccctgccgcttaccggat
acctgtccgcctttctcccttcgggaagcgtggcgctttctcaatgctcacgctgtaggtatctcagttcggtgtaggtcgttcg
ctccaagctgggctgtgtgcacgaaccccccgttcagcccgaccgctgcgccttatccggtaactatcgtcttgagtccaacccg
gtaagacacgacttatcgccactggcagcagccactggtaacaggattagcagagcgaggtatgtaggcggtgctacagag
ttcttgaagtggtggcctaactacggctacactagaaggacagtatttggtatctgcgctctgctgaagccagttaccttcgga
aaaagagttggtagctcttgatccggcaaacaaaccaccgctggtagcggtggtttttttgtttgcaagcagcagattacgcg
cagaaaaaaaggatctcaagaagatcctttgatcttttctacggggtctgacgctcagtggaacgaaaactcacgttaaggg
attttggtcatgagattatcaaaaaggatcttcacctagatccttttgggggggcgaagaactccagcatgagatccccgcg
ctggaggatcatccagccggcgtcccggaaaacgattccgaagcccaacctttcatagaaggcggcggtggaatcgaaatct
cgtgatggcaggttgggcgtcgcttggtcggtcatttcgaaccccagagtcccgctcagaagaactcgtcaagaaggcgata
gaaggcgatgcgctgcgaatcgggagcggcgataccgtaaagcacgaggaagcggtcagcccattcgccgccaagctcttc
agcaatatcacgggtagccaacgctatgtcctgatagcggtccgccacacccagccggccacagtcgatgaatccagaaaa
gcggccattttccaccatgatattcggcaagcaggcatcgccatgggtcacgacgagatcctcgccgtcgggcatccgcgcct
tgagcctggcgaacagttcggctggcgcgagcccctgatgctcttcgtccagatcatcctgatcgacaagaccggcttccatc
cgagtacgtgctcgctcgatgcgatgtttcgcttggtggtcgaatgggcaggtagccggatcaagcgtatgcagccgccgcat
tgcatcagccatgatggatactttctcggcaggagcaaggtgagatgacaggagatcctgccccggcacttcgcccaatagc
agccagtcccttcccgcttcagtgacaacgtcgagcacagctgcgcaaggaacgcccgtcgtggccagccacgatagccgcg
ctgcctcgtcttggagttcattcagggcaccggacaggtcggtcttgacaaaaagaaccgggcgcccctgcgctgacagccg
gaacacggcggcatcagagcagccgattgtctgttgtgcccagtcatagccgaatagcctctccacccaagcggccggagaa
cctgcgtgcaatccatcttgttcaatcatgcgaaacgatcctcatcctgtctcttgatcagatcttgatcccctgcgccatcaga
tccttggcggcaagaaagccatccagtttactttgcagggcttcccaaccttaccagagggcgccccagctggcaattccggttc
gcttgctgtccataaaaccgcccagtctagctatcgccatgtaagcccactgcaagctacctgctttctctttgcgcttgcgttt
tcccttgtccagatagcccagtagctgacattcatccggggtcagcaccgtttctgcggactggctttctacgtgttccgcttcc
tttagcagcccttgcgccctgagtgcttgcggcagcgtgaagctagcgtctaccaggacttttacctgtccgacccgttgcaacg
gaaccccccacggaacccccgcgacacccgctccccaattgcgttagaacagcggtggattgtcggcttcgttgtgggcctttt
gagccgcttcctgttctgccgcacgctctttcctcgcccgatagccgagtcgcttaacggtgtccagatgcagcccgaaatgttt
ggccgtttgcggccaagagtggccctcgtcgtcgtgataggcgcggatgcgttcgcggcgtgcagcctgctcggcgagccact
cgctgcgttcctgcgccacgagccggacgacgtggcgttcggatagtccggtgattcgagcgccttcggcggcggtcacgcgc
cgctttttgcggacagtcggctgccggttgtagccgtcgctgtagccgtcgctcatagcaatgcctccatggctgacgcggact
ttgcgcgccgcgcaactgtgctcgccgccgtgcgcgctgctgcgcccttccgcgagatggccgactggcgcgcactgagtgtg
gcctcgtagaccacgatcccgtccgcccaaatgcgcgacttggttgtgatccaacgccaaatgctgttggcgatggcgcggac
ctcgctgtccggtagcggtccgggacacacgtcgttgcacgggaactcggcgtttcgcgcgtggcactcggcatagatcgcgc
ggccgagtccgtccacgttccgggtcggcaggtagatccgcatgagggcgggacgataggcccacaacctgacggaatcga
acagtgcgcaattccgccctagcggcgtcggagccgctttgtacgtggtctgctgacgccagcgcggcggtggcatgttcgcg
ccgagctcggcctcgatgtggctgagtgtgtagagatctgagtggagccattccgtttcccaggcgatgtggccggggtttttg
gtcatgaggcctgagtaactgcggtcgccgtccacggcgcgccgaaggccttcggcgcacgccgccatgtatgcgagcggct
tacgccgcgcgtattcggtgcgtggaacaggggcgttgagtgcccacactgcgtgtgcgtggccgttggcgcgattgcccacg
atcgcgttgggcagcggatgggacccccgggcgctgagcgctcggagcgctgcgtctggatggtctacgtccacgaccagca
ggtttgccagcgctgttgggttcgcctcgatgtaccggcggcctagggccgacgcgcggctttggcggtagatcccctcgagc
agatcgtcgcttgccagcggccagtacggcagccagagctgctcaaattcgtcggcgacgtggctcacgcttggtagtagac
cacgattaatcaccggtgtatggtccgacacgagctccaagtcagatatttcgctgaggggccaccccacaactgcacactcc
cccgctctcccgtcgagccctggtggtggaacaccagcgacagccgagcacccccaaccacctgtaccaacc
pGZ003 (SEQ ID NO: 4)
actagttgattagctaagcagaaggccatcctgacggatggcctttttgcgtttaatactgtttaaactctagaaatattggatc
gtcggcaccgtcacggccgtgggaggcggcacgatccgcgacgtgatgatcggccgcatccccacggtgctgcgcagtgag
ctctacgccatcccggcgttgatctgtgcgttcgctcgcgtcagcttaaggttccgttcaccggtcggacgaattctcacgacat
ccagtgtcacctcgtcagcattttttcgggtgcccgaactttcctgtgagggaatcgctcggtgaccccaactgttgcgcagggt
gtaccaatgagattgggtatatacccgagaacgattcgccaaaggcgggtgtcagacgcgtgcccacgtgtcgtcggcaccc
ggaaacacaagggctcacacgcccatagatgaagggaccctcatggctagcaaaggagaagaacttttcactggagttgtc
ccaattcttgttgaattagatggtgatgttaatgggcacaaattttctgtcagtggagagggtgaaggtgatgctacatacgga
aagcttacccttaaatttatttgcactactggaaaactacctgttccatggccaacacttgtcactactttgacctatggtgttc
aatgcttttcccgttatccggatcatatgaaacggcatgactttttcaagagtgccatgcccgaaggttatgtacaggaacgcac
tatatctttcaaagatgacgggaactacaagacgcgtgctgaagtcaagtttgaaggtgatacccttgttaatcgtatcgagtt
aaaaggtattgattttaaagaagatggaaacattctcggacacaaactcgagtacaactataactcacacaatgtatacatc
acggcagacaaacaaaagaatggaatcaaagctaacttcaaaattcgccacaacattgaagatggctccgttcaactagca
gaccattatcaacaaaatactccaattggcgatggccctgtccttttaccagacaaccattacctgtcgacacaatctgcccttt
cgaaagatcccaacgaaaagcgtgaccacatggtccttcttgagtttgtaactgctgctgggattacacatggcatggatgag
ctctacaaataatgaattcgagctggatccagcttcctcgctcactgactcgctgcgctcggtcgttcggctgcggcgagcggt
atcagctcactcaaaggcggtaatacggttatccacagaatcaggggataacgcaggaaagaacatgtgagcaaaaggcc
agcaaaaggccaggaaccgtaaaaaggccgcgttgctggcgtttttccataggctccgcccccctgacgagcatcacaaaa
atcgacgctcaagtcagaggtggcgaaacccgacaggactataaagataccaggcgtttccccctggaagctccctcgtgcg
ctctcctgttccgaccctgccgcttaccggatacctgtccgcctttctcccttcgggaagcgtggcgctttctcaatgctcacgc
tgtaggtatctcagttcggtgtaggtcgttcgctccaagctgggctgtgtgcacgaaccccccgttcagcccgaccgctgcgcct
tatccggtaactatcgtcttgagtccaacccggtaagacacgacttatcgccactggcagcagccactggtaacaggattagc
agagcgaggtatgtaggcggtgctacagagttcttgaagtggtggcctaactacggctacactagaaggacagtatttggtat
ctgcgctctgctgaagccagttaccttcggaaaaagagttggtagctcttgatccggcaaacaaaccaccgctggtagcggtg
gtttttttgtttgcaagcagcagattacgcgcagaaaaaaaggatctcaagaagatcctttgatcttttctacggggtctgacg
ctcagtggaacgaaaactcacgttaagggattttggtcatgagattatcaaaaaggatcttcacctagatccttttggggtggg
cgaagaactccagcatgagatccccgcgctggaggatcatccagccggcgtcccggaaaacgattccgaagcccaacctttc
atagaaggcggcggtggaatcgaaatctcgtgatggcaggttgggcgtcgcttggtcggtcatttcgaaccccagagtcccgc
tcagaagaactcgtcaagaaggcgatagaaggcgatgcgctgcgaatcgggagcggcgataccgtaaagcacgaggaag
cggtcagcccattcgccgccaagctcttcagcaatatcacgggtagccaacgctatgtcctgatagcggtccgccacacccag
ccggccacagtcgatgaatccagaaaagcggccattttccaccatgatattcggcaagcaggcatcgccatgggtcacgacg
agatcctcgccgtcgggcatccgcgccttgagcctggcgaacagttcggctggcgcgagcccctgatgctcttcgtccagatc
atcctgatcgacaagaccggcttccatccgagtacgtgctcgctcgatgcgatgtttcgcttggtggtcgaatgggcaggtagc
cggatcaagcgtatgcagccgccgcattgcatcagccatgatggatactttctcggcaggagcaaggtgagatgacaggag
atcctgccccggcacttcgcccaatagcagccagtcccttcccgcttcagtgacaacgtcgagcacagctgcgcaaggaacg
cccgtcgtggccagccacgatagccgcgctgcctcgtcttggagttcattcagggcaccggacaggtcggtcttgacaaaaag
aaccgggcgcccctgcgctgacagccggaacacggcggcatcagagcagccgattgtctgttgtgcccagtcatagccgaat
agcctctccacccaagcggccggagaacctgcgtgcaatccatcttgttcaatcatgcgaaacgatcctcatcctgtctcttga
tcagatcttgatcccctgcgccatcagatccttggcggcaagaaagccatccagtttactttgcagggcttcccaaccttacca
gagggcgccccagctggcaattccggttcgcttgctgtccataaaaccgcccagtctagctatcgccatgtaagcccactgcaa
gctacctgctttctctttgcgcttgcgttttcccttgtccagatagcccagtagctgacattcatccggggtcagcaccgtttct
gcggactggctttctacgtgttccgcttcctttagcagcccttgcgccctgagtgcttgcggcagcgtgaagctagcgtctacca
ggacttttacctgtccgacccgttgcaacggaaccccccacggaacccccgcgacacccgctccccaattgcgttagaacagc
ggtggattgtcggcttcgttgtgggccttttgagccgcttcctgttctgccgcacgctctttcctcgcccgatagccgagtcgct
taacggtgtccagatgcagcccgaaatgtttggccgtttgcggccaagagtggccctcgtcgtcgtgataggcgcggatgcgtt
cgcggcgtgcagcctgctcggcgagccactcgctgcgttcctgcgccacgagccggacgacgtggcgttcggatagtccggt
gattcgagcgccttcggcggcggtcacgcgccgctttttgcggacagtcggctgccggttgtagccgtcgctgtagccgtcgct
catagcaatgcctccatggctgacgcggactttgcgcgccgcgcaactgtgctcgccgccgtgcgcgctgctgcgcccttccgc
gagatggccgactggcgcgcactgagtgtggcctcgtagaccacgatcccgtccgcccaaatgcgcgacttggttgtgatcca
acgccaaatgctgttggcgatggcgcggacctcgctgtccggtagcggtccgggacacacgtcgttgcacgggaactcggcg
tttcgcgcgtggcactcggcatagatcgcgcggccgagtccgtccacgttccgggtcggcaggtagatccgcatgagggcgg
gacgataggcccacaacctgacggaatcgaacagtgcgcaattccgccctagcggcgtcggagccgctttgtacgtggtctg
ctgacgccagcgcggcggtggcatgttcgcgccgagctcggcctcgatgtggctgagtgtgtagagatctgagtggagccatt
ccgtttcccaggcgatgtggccggggtttttggtcatgaggcctgagtaactgcggtcgccgtccacggcgcgccgaaggcct
tcggcgcacgccgccatgtatgcgagcggcttacgccgcgcgtattcggtgcgtggaacaggggcgttgagtgcccacactg
cgtgtgcgtggccgttggcgcgattgcccacgatcgcgttgggcagcggatgggacccccgggcgctgagcgctcggagcgc
tgcgtctggatggtctacgtccacgaccagcaggtttgccagcgctgttgggttcgcctcgatgtaccggcggcctagggccg
acgcgcggctttggcggtagatcccctcgagcagatcgtcgcttgccagcggccagtacggcagccagagctgctcaaattc
gtcggcgacgtggctcacgcttggtagtagaccacgattaatcaccggtgtatggtccgacacgagctccaagtcagatattt
cgctgaggggccaccccacaactgcacactcccccgctctcccgtcgagccctggtggtggaacaccagcgacagccgagc
acccccaaccacctgtaccaacc
pGZ004 (SEQ ID NO: 5)
actagttgattagctaagcagaaggccatcctgacggatggcctttttgcgtttaatactgtttaaactctagaaatattggatc
gtcggcaccgtcacggccgtgggaggcggcacgatccgcgacgtgatgatcggccgcatccccacggtgctgcgcagtgag
ctctacgccatcccggcgttgatctgtgcgttcgctgctgcgatcctatcgacctgccgtagcgtggcgtgccgacgcgcgggt
ccgcgtcggcggcctcgctacgggaaggcctcgaaacaaggcgccgaaaaaagatgtgggcatccgggaacaaatctccg
cacccctccgttgagccttacgacaacatgagcgtgcaagactcaagttcgaattgactcccgacggtgtcggagtgcaaact
tgagcggagggcgctcactaagcgccaacatcgcagttcagtaatgaaaaacctgcccacaggactgcaaaacgaaagtg
aggaacactatggctagcaaaggagaagaacttttcactggagttgtcccaattcttgttgaattagatggtgatgttaatgg
gcacaaattttctgtcagtggagagggtgaaggtgatgctacatacggaaagcttacccttaaatttatttgcactactggaa
aactacctgttccatggccaacacttgtcactactttgacctatggtgttcaatgcttttcccgttatccggatcatatgaaacg
gcatgactttttcaagagtgccatgcccgaaggttatgtacaggaacgcactatatctttcaaagatgacgggaactacaagac
gcgtgctgaagtcaagtttgaaggtgatacccttgttaatcgtatcgagttaaaaggtattgattttaaagaagatggaaacat
tctcggacacaaactcgagtacaactataactcacacaatgtatacatcacggcagacaaacaaaagaatggaatcaaagc
taacttcaaaattcgccacaacattgaagatggctccgttcaactagcagaccattatcaacaaaatactccaattggcgatg
gccctgtccttttaccagacaaccattacctgtcgacacaatctgccctttcgaaagatcccaacgaaaagcgtgaccacatg
gtccttcttgagtttgtaactgctgctgggattacacatggcatggatgagctctacaaataatgaattcgagctggatccagc
ttcctcgctcactgactcgctgcgctcggtcgttcggctgcggcgagcggtatcagctcactcaaaggcggtaatacggttatc
cacagaatcaggggataacgcaggaaagaacatgtgagcaaaaggccagcaaaaggccaggaaccgtaaaaaggccgc
gttgctggcgtttttccataggctccgcccccctgacgagcatcacaaaaatcgacgctcaagtcagaggtggcgaaacccga
caggactataaagataccaggcgtttccccctggaagctccctcgtgcgctctcctgttccgaccctgccgcttaccggatacc
tgtccgcctttctcccttcgggaagcgtggcgctttctcaatgctcacgctgtaggtatctcagttcggtgtaggtcgttcgctc
caagctgggctgtgtgcacgaaccccccgttcagcccgaccgctgcgccttatccggtaactatcgtcttgagtccaacccggta
agacacgacttatcgccactggcagcagccactggtaacaggattagcagagcgaggtatgtaggcggtgctacagagttct
tgaagtggtggcctaactacggctacactagaaggacagtatttggtatctgcgctctgctgaagccagttaccttcggaaaa
agagttggtagctcttgatccggcaaacaaaccaccgctggtagcggtggtttttttgtttgcaagcagcagattacgcgcag
aaaaaaaggatctcaagaagatcctttgatcttttctacggggtctgacgctcagtggaacgaaaactcacgttaagggattt
tggtcatgagattatcaaaaaggatcttcacctagatccttttggggtgggcgaagaactccagcatgagatccccgcgctgg
aggatcatccagccggcgtcccggaaaacgattccgaagcccaacctttcatagaaggcggcggtggaatcgaaatctcgtg
atggcaggttgggcgtcgcttggtcggtcatttcgaaccccagagtcccgctcagaagaactcgtcaagaaggcgatagaag
gcgatgcgctgcgaatcgggagcggcgataccgtaaagcacgaggaagcggtcagcccattcgccgccaagctcttcagca
atatcacgggtagccaacgctatgtcctgatagcggtccgccacacccagccggccacagtcgatgaatccagaaaagcgg
ccattttccaccatgatattcggcaagcaggcatcgccatgggtcacgacgagatcctcgccgtcgggcatccgcgccttgag
cctggcgaacagttcggctggcgcgagcccctgatgctcttcgtccagatcatcctgatcgacaagaccggcttccatccgag
tacgtgctcgctcgatgcgatgtttcgcttggtggtcgaatgggcaggtagccggatcaagcgtatgcagccgccgcattgcat
cagccatgatggatactttctcggcaggagcaaggtgagatgacaggagatcctgccccggcacttcgcccaatagcagcca
gtcccttcccgcttcagtgacaacgtcgagcacagctgcgcaaggaacgcccgtcgtggccagccacgatagccgcgctgcc
tcgtcttggagttcattcagggcaccggacaggtcggtcttgacaaaaagaaccgggcgcccctgcgctgacagccggaaca
cggcggcatcagagcagccgattgtctgttgtgcccagtcatagccgaatagcctctccacccaagcggccggagaacctgc
gtgcaatccatcttgttcaatcatgcgaaacgatcctcatcctgtctcttgatcagatcttgatcccctgcgccatcagatcctt
ggcggcaagaaagccatccagtttactttgcagggcttcccaaccttaccagagggcgccccagctggcaattccggttcgcttg
ctgtccataaaaccgcccagtctagctatcgccatgtaagcccactgcaagctacctgctttctctttgcgcttgcgttttccct
tgtccagatagcccagtagctgacattcatccggggtcagcaccgtttctgcggactggctttctacgtgttccgcttcctttag
cagcccttgcgccctgagtgcttgcggcagcgtgaagctagcgtctaccaggacttttacctgtccgacccgttgcaacggaac
cccccacggaacccccgcgacacccgctccccaattgcgttagaacagcggtggattgtcggcttcgttgtgggccttttgagc
cgcttcctgttctgccgcacgctctttcctcgcccgatagccgagtcgcttaacggtgtccagatgcagcccgaaatgtttggcc
gtttgcggccaagagtggccctcgtcgtcgtgataggcgcggatgcgttcgcggcgtgcagcctgctcggcgagccactcgct
gcgttcctgcgccacgagccggacgacgtggcgttcggatagtccggtgattcgagcgccttcggcggcggtcacgcgccgct
ttttgcggacagtcggctgccggttgtagccgtcgctgtagccgtcgctcatagcaatgcctccatggctgacgcggactttgc
gcgccgcgcaactgtgctcgccgccgtgcgcgctgctgcgcccttccgcgagatggccgactggcgcgcactgagtgtggcct
cgtagaccacgatcccgtccgcccaaatgcgcgacttggttgtgatccaacgccaaatgctgttggcgatggcgcggacctcg
ctgtccggtagcggtccgggacacacgtcgttgcacgggaactcggcgtttcgcgcgtggcactcggcatagatcgcgcggc
cgagtccgtccacgttccgggtcggcaggtagatccgcatgaggggggacgataggcccacaacctgacggaatcgaaca
gtgcgcaattccgccctagcggcgtcggagccgctttgtacgtggtctgctgacgccagcgcggcggtggcatgttcgcgccg
agctcggcctcgatgtggctgagtgtgtagagatctgagtggagccattccgtttcccaggcgatgtggccggggtttttggtc
atgaggcctgagtaactgcggtcgccgtccacggcgcgccgaaggccttcggcgcacgccgccatgtatgcgagcggcttac
gccgcgcgtattcggtgcgtggaacaggggcgttgagtgcccacactgcgtgtgcgtggccgttggcgcgattgcccacgatc
gcgttgggcagcggatgggacccccgggcgctgagcgctcggagcgctgcgtctggatggtctacgtccacgaccagcaggt
ttgccagcgctgttgggttcgcctcgatgtaccggcggcctagggccgacgcgcggctttggcggtagatcccctcgagcaga
tcgtcgcttgccagcggccagtacggcagccagagctgctcaaattcgtcggcgacgtggctcacgcttggtagtagaccacg
attaatcaccggtgtatggtccgacacgagctccaagtcagatatttcgctgaggggccaccccacaactgcacactcccccg
ctctcccgtcgagccctggtggtggaacaccagcgacagccgagcacccccaaccacctgtaccaacc
pGZ005 (SEQ ID NO: 6)
actagttgattagctaagcagaaggccatcctgacggatggcctttttgcgtttaatactgtttaaactctagaaatattggatc
gtcggcaccgtcacggccgtgggaggcggcacgatccgcgacgtgatgatcggccgcatccccacggtgctgcgcagtgag
ctctacgccatcccggcgttgatctgtgcgttcgctgaggaaaagctagctcatatatgttacgtctcgtaacagataatgggc
gcagaggcagtctgatgaaccacacccccgcatgccgagtgacgccggttactatgacggtcggggtccggccgcgatttgg
cggttgtgcaggccatgccgtaatctgtcctgcggagttcaatgcagtagttgaacttcaccccaaatcatgttctacccaaga
ccgttggtgatcactcccgcaagggtgtgattgaaagtccgggaatgtccggcggcccacgcaggaggacgaggtaggggg
atgcgtatcggttgagggctcgcgccctgaccgtatcgtttcctttcacgccccgtgtgccctgcaccggggcgttttgcatgtc
gtggattgtgtgctccgggattccggcggtgcgcagcggttccgaagtgggataacgactgtcatgagaggaggcgaagtat
ggctagcaaaggagaagaacttttcactggagttgtcccaattcttgttgaattagatggtgatgttaatgggcacaaattttc
tgtcagtggagagggtgaaggtgatgctacatacggaaagcttacccttaaatttatttgcactactggaaaactacctgttcc
atggccaacacttgtcactactttgacctatggtgttcaatgcttttcccgttatccggatcatatgaaacggcatgactttttc
aagagtgccatgcccgaaggttatgtacaggaacgcactatatctttcaaagatgacgggaactacaagacgcgtgctgaagt
caagtttgaaggtgatacccttgttaatcgtatcgagttaaaaggtattgattttaaagaagatggaaacattctcggacaca
aactcgagtacaactataactcacacaatgtatacatcacggcagacaaacaaaagaatggaatcaaagctaacttcaaaa
ttcgccacaacattgaagatggctccgttcaactagcagaccattatcaacaaaatactccaattggcgatggccctgtccttt
taccagacaaccattacctgtcgacacaatctgccctttcgaaagatcccaacgaaaagcgtgaccacatggtccttcttgag
tttgtaactgctgctgggattacacatggcatggatgagctctacaaataatgaattcgagctggatccagcttcctcgctcac
tgactcgctgcgctcggtcgttcggctgcggcgagcggtatcagctcactcaaaggcggtaatacggttatccacagaatcag
gggataacgcaggaaagaacatgtgagcaaaaggccagcaaaaggccaggaaccgtaaaaaggccgcgttgctggcgttt
ttccataggctccgcccccctgacgagcatcacaaaaatcgacgctcaagtcagaggtggcgaaacccgacaggactataaa
gataccaggcgtttccccctggaagctccctcgtgcgctctcctgttccgaccctgccgcttaccggatacctgtccgcctttct
cccttcgggaagcgtggcgctttctcaatgctcacgctgtaggtatctcagttcggtgtaggtcgttcgctccaagctgggctgt
gtgcacgaaccccccgttcagcccgaccgctgcgccttatccggtaactatcgtcttgagtccaacccggtaagacacgactt
atcgccactggcagcagccactggtaacaggattagcagagcgaggtatgtaggcggtgctacagagttcttgaagtggtgg
cctaactacggctacactagaaggacagtatttggtatctgcgctctgctgaagccagttaccttcggaaaaagagttggtag
ctcttgatccggcaaacaaaccaccgctggtagcggtggtttttttgtttgcaagcagcagattacgcgcagaaaaaaaggat
ctcaagaagatcctttgatcttttctacggggtctgacgctcagtggaacgaaaactcacgttaagggattttggtcatgagat
tatcaaaaaggatcttcacctagatccttttggggtgggcgaagaactccagcatgagatccccgcgctggaggatcatccag
ccggcgtcccggaaaacgattccgaagcccaacctttcatagaaggggcggtggaatcgaaatctcgtgatggcaggttgg
gcgtcgcttggtcggtcatttcgaaccccagagtcccgctcagaagaactcgtcaagaaggcgatagaaggcgatgcgctgc
gaatcgggagcggcgataccgtaaagcacgaggaagcggtcagcccattcgccgccaagctcttcagcaatatcacgggta
gccaacgctatgtcctgatagcggtccgccacacccagccggccacagtcgatgaatccagaaaagcggccattttccacca
tgatattcggcaagcaggcatcgccatgggtcacgacgagatcctcgccgtcgggcatccgcgccttgagcctggcgaacag
ttcggctggcgcgagcccctgatgctcttcgtccagatcatcctgatcgacaagaccggcttccatccgagtacgtgctcgctc
gatgcgatgtttcgcttggtggtcgaatgggcaggtagccggatcaagcgtatgcagccgccgcattgcatcagccatgatgg
atactttctcggcaggagcaaggtgagatgacaggagatcctgccccggcacttcgcccaatagcagccagtcccttcccgct
tcagtgacaacgtcgagcacagctgcgcaaggaacgcccgtcgtggccagccacgatagccgcgctgcctcgtcttggagtt
cattcagggcaccggacaggtcggtcttgacaaaaagaaccgggcgcccctgcgctgacagccggaacacggcggcatca
gagcagccgattgtctgttgtgcccagtcatagccgaatagcctctccacccaagcggccggagaacctgcgtgcaatccatc
ttgttcaatcatgcgaaacgatcctcatcctgtctcttgatcagatcttgatcccctgcgccatcagatccttggcggcaagaaa
gccatccagtttactttgcagggcttcccaaccttaccagagggcgccccagctggcaattccggttcgcttgctgtccataaaa
ccgcccagtctagctatcgccatgtaagcccactgcaagctacctgctttctctttgcgcttgcgttttcccttgtccagatagc
ccagtagctgacattcatccggggtcagcaccgtttctgcggactggctttctacgtgttccgcttcctttagcagcccttgcgc
cctgagtgcttgcggcagcgtgaagctagcgtctaccaggacttttacctgtccgacccgttgcaacggaaccccccacggaac
ccccgcgacacccgctccccaattgcgttagaacagcggtggattgtcggcttcgttgtgggccttttgagccgcttcctgttct
gccgcacgctctttcctcgcccgatagccgagtcgcttaacggtgtccagatgcagcccgaaatgtttggccgtttgcggccaa
gagtggccctcgtcgtcgtgataggcgcggatgcgttcgcggcgtgcagcctgctcggcgagccactcgctgcgttcctgcgc
cacgagccggacgacgtggcgttcggatagtccggtgattcgagcgccttcggcggcggtcacgcgccgctttttgcggacag
tcggctgccggttgtagccgtcgctgtagccgtcgctcatagcaatgcctccatggctgacgcggactttgcgcgccgcgcaac
tgtgctcgccgccgtgcgcgctgctgcgcccttccgcgagatggccgactggcgcgcactgagtgtggcctcgtagaccacga
tcccgtccgcccaaatgcgcgacttggttgtgatccaacgccaaatgctgttggcgatggcgcggacctcgctgtccggtagc
ggtccgggacacacgtcgttgcacgggaactcggcgtttcgcgcgtggcactcggcatagatcgcgcggccgagtccgtcca
cgttccgggtcggcaggtagatccgcatgagggcgggacgataggcccacaacctgacggaatcgaacagtgcgcaattcc
gccctagcggcgtcggagccgctttgtacgtggtctgctgacgccagcgcggcggtggcatgttcgcgccgagctcggcctcg
atgtggctgagtgtgtagagatctgagtggagccattccgtttcccaggcgatgtggccggggtttttggtcatgaggcctgag
taactgcggtcgccgtccacggcgcgccgaaggccttcggcgcacgccgccatgtatgcgagcggcttacgccgcgcgtattc
ggtgcgtggaacaggggcgttgagtgcccacactgcgtgtgcgtggccgttggcgcgattgcccacgatcgcgttgggcagc
ggatgggacccccgggcgctgagcgctcggagcgctgcgtctggatggtctacgtccacgaccagcaggtttgccagcgctg
ttgggttcgcctcgatgtaccggcggcctagggccgacgcgcggctttggcggtagatcccctcgagcagatcgtcgcttgcc
agcggccagtacggcagccagagctgctcaaattcgtcggcgacgtggctcacgcttggtagtagaccacgattaatcaccg
gtgtatggtccgacacgagctccaagtcagatatttcgctgaggggccaccccacaactgcacactcccccgctctcccgtcg
agccctggtggtggaacaccagcgacagccgagcacccccaaccacctgtaccaacc
pGZ006 (SEQ ID NO: 7)
actagttgattagctaagcagaaggccatcctgacggatggcctttttgcgtttaatactgtttaaactctagaaatattggatc
gtcggcaccgtcacggccgtgggaggcggcacgatccgcgacgtgatgatcggccgcatccccacggtgctgcgcagtgag
ctctacgccatcccggcgttgatctgtgcgttcgctcaggtctgcgcgatgcgctgatcgcccgggcgcaccggttccgtcccg
accttgcactcaccctgtccgagtgctaaaaatgcacttggcactcacgacgcgtgagtgccaggtcgggacggtgagaccg
ggaaccaaagacacccctggtcgtccgtcgcgggcaccgaactcggccgaaggcgtaaatgaggcgacccgactagcggtc
gccttgtgtgtcacccccaatccggaggatcacttcgcaatggctagcaaaggagaagaacttttcactggagttgtcccaatt
cttgttgaattagatggtgatgttaatgggcacaaattttctgtcagtggagagggtgaaggtgatgctacatacggaaagctt
acccttaaatttatttgcactactggaaaactacctgttccatggccaacacttgtcactactttgacctatggtgttcaatgct
tttcccgttatccggatcatatgaaacggcatgactttttcaagagtgccatgcccgaaggttatgtacaggaacgcactatatc
tttcaaagatgacgggaactacaagacgcgtgctgaagtcaagtttgaaggtgatacccttgttaatcgtatcgagttaaaag
gtattgattttaaagaagatggaaacattctcggacacaaactcgagtacaactataactcacacaatgtatacatcacggca
gacaaacaaaagaatggaatcaaagctaacttcaaaattcgccacaacattgaagatggctccgttcaactagcagaccatt
atcaacaaaatactccaattggcgatggccctgtccttttaccagacaaccattacctgtcgacacaatctgccctttcgaaag
atcccaacgaaaagcgtgaccacatggtccttcttgagtttgtaactgctgctgggattacacatggcatggatgagctctaca
aataatgaattcgagctggatccagcttcctcgctcactgactcgctgcgctcggtcgttcggctgcggcgagcggtatcagct
cactcaaaggcggtaatacggttatccacagaatcaggggataacgcaggaaagaacatgtgagcaaaaggccagcaaaa
ggccaggaaccgtaaaaaggccgcgttgctggcgtttttccataggctccgcccccctgacgagcatcacaaaaatcgacgc
tcaagtcagaggtggcgaaacccgacaggactataaagataccaggcgtttccccctggaagctccctcgtgcgctctcctgt
tccgaccctgccgcttaccggatacctgtccgcctttctcccttcgggaagcgtggcgctttctcaatgctcacgctgtaggtat
ctcagttcggtgtaggtcgttcgctccaagctgggctgtgtgcacgaaccccccgttcagcccgaccgctgcgccttatccggt
aactatcgtcttgagtccaacccggtaagacacgacttatcgccactggcagcagccactggtaacaggattagcagagcga
ggtatgtaggcggtgctacagagttcttgaagtggtggcctaactacggctacactagaaggacagtatttggtatctgcgctc
tgctgaagccagttaccttcggaaaaagagttggtagctcttgatccggcaaacaaaccaccgctggtagcggtggtttttttg
tttgcaagcagcagattacgcgcagaaaaaaaggatctcaagaagatcctttgatcttttctacggggtctgacgctcagtgg
aacgaaaactcacgttaagggattttggtcatgagattatcaaaaaggatcttcacctagatccttttggggtgggcgaagaa
ctccagcatgagatccccgcgctggaggatcatccagccggcgtcccggaaaacgattccgaagcccaacctttcatagaag
gcggcggtggaatcgaaatctcgtgatggcaggttgggcgtcgcttggtcggtcatttcgaaccccagagtcccgctcagaag
aactcgtcaagaaggcgatagaaggcgatgcgctgcgaatcgggagcggcgataccgtaaagcacgaggaagcggtcagc
ccattcgccgccaagctcttcagcaatatcacgggtagccaacgctatgtcctgatagcggtccgccacacccagccggccac
agtcgatgaatccagaaaagcggccattttccaccatgatattcggcaagcaggcatcgccatgggtcacgacgagatcctc
gccgtcgggcatccgcgccttgagcctggcgaacagttcggctggcgcgagcccctgatgctcttcgtccagatcatcctgatc
gacaagaccggcttccatccgagtacgtgctcgctcgatgcgatgtttcgcttggtggtcgaatgggcaggtagccggatcaa
gcgtatgcagccgccgcattgcatcagccatgatggatactttctcggcaggagcaaggtgagatgacaggagatcctgccc
cggcacttcgcccaatagcagccagtcccttcccgcttcagtgacaacgtcgagcacagctgcgcaaggaacgcccgtcgtg
gccagccacgatagccgcgctgcctcgtcttggagttcattcagggcaccggacaggtcggtcttgacaaaaagaaccgggc
gcccctgcgctgacagccggaacacggcggcatcagagcagccgattgtctgttgtgcccagtcatagccgaatagcctctcc
acccaagcggccggagaacctgcgtgcaatccatcttgttcaatcatgcgaaacgatcctcatcctgtctcttgatcagatctt
gatcccctgcgccatcagatccttggcggcaagaaagccatccagtttactttgcagggcttcccaaccttaccagagggcgc
cccagctggcaattccggttcgcttgctgtccataaaaccgcccagtctagctatcgccatgtaagcccactgcaagctacctgc
tttctctttgcgcttgcgttttcccttgtccagatagcccagtagctgacattcatccggggtcagcaccgtttctgcggactgg
ctttctacgtgttccgcttcctttagcagcccttgcgccctgagtgcttgcggcagcgtgaagctagcgtctaccaggactttta
cctgtccgacccgttgcaacggaaccccccacggaacccccgcgacacccgctccccaattgcgttagaacagcggtggattg
tcggcttcgttgtgggccttttgagccgcttcctgttctgccgcacgctctttcctcgcccgatagccgagtcgcttaacggtgt
ccagatgcagcccgaaatgtttggccgtttgcggccaagagtggccctcgtcgtcgtgataggcgcggatgcgttcgcggcgtg
cagcctgctcggcgagccactcgctgcgttcctgcgccacgagccggacgacgtggcgttcggatagtccggtgattcgagcg
ccttcggcggcggtcacgcgccgctttttgcggacagtcggctgccggttgtagccgtcgctgtagccgtcgctcatagcaatg
cctccatggctgacgcggactttgcgcgccgcgcaactgtgctcgccgccgtgcgcgctgctgcgcccttccgcgagatggcc
gactggcgcgcactgagtgtggcctcgtagaccacgatcccgtccgcccaaatgcgcgacttggttgtgatccaacgccaaat
gctgttggcgatggcgcggacctcgctgtccggtagcggtccgggacacacgtcgttgcacgggaactcggcgtttcgcgcgt
ggcactcggcatagatcgcgcggccgagtccgtccacgttccgggtcggcaggtagatccgcatgagggcgggacgatagg
cccacaacctgacggaatcgaacagtgcgcaattccgccctagcggcgtcggagccgctttgtacgtggtctgctgacgcca
gcgcggcggtggcatgttcgcgccgagctcggcctcgatgtggctgagtgtgtagagatctgagtggagccattccgtttccc
aggcgatgtggccggggtttttggtcatgaggcctgagtaactgcggtcgccgtccacggcgcgccgaaggccttcggcgca
cgccgccatgtatgcgagcggcttacgccgcgcgtattcggtgcgtggaacaggggcgttgagtgcccacactgcgtgtgcgt
ggccgttggcgcgattgcccacgatcgcgttgggcagcggatgggacccccgggcgctgagcgctcggagcgctgcgtctgg
atggtctacgtccacgaccagcaggtttgccagcgctgttgggttcgcctcgatgtaccggcggcctagggccgacgcgcggc
tttggcggtagatcccctcgagcagatcgtcgcttgccagcggccagtacggcagccagagctgctcaaattcgtcggcgacg
tggctcacgcttggtagtagaccacgattaatcaccggtgtatggtccgacacgagctccaagtcagatatttcgctgagggg
ccaccccacaactgcacactcccccgctctcccgtcgagccctggtggtggaacaccagcgacagccgagcacccccaacca
cctgtaccaacc
pGZ007 (SEQ ID NO: 8)
actagttgattagctaagcagaaggccatcctgacggatggcctttttgcgtttaatactgtttaaactctagaaatattggatc
gtcggcaccgtcacggccgtgggaggcggcacgatccgcgacgtgatgatcggccgcatccccacggtgctgcgcagtgag
ctctacgccatcccggcgttgatctgtgcgttcgctgcacaccatccttcgcatcgtggccgtgggtatcggaagtcgaattctg
ccgggcgggcggctccgtcggggaccgcatttggcctggtgagcaatttcccgtaacatggatcaacggtgcgctatgcgtgc
cggttgtctgcgtgcccagtgccggattcgatccggattgttcatgtttcggccggtgaagatcccattgggcgccacggtaac
ggaaacagatgcaataagcacgccaaaccacatggatcgcggaggatatggctagcaaaggagaagaacttttcactgga
gttgtcccaattcttgttgaattagatggtgatgttaatgggcacaaattttctgtcagtggagagggtgaaggtgatgctacat
acggaaagcttacccttaaatttatttgcactactggaaaactacctgttccatggccaacacttgtcactactttgacctatgg
tgttcaatgcttttcccgttatccggatcatatgaaacggcatgactttttcaagagtgccatgcccgaaggttatgtacaggaa
cgcactatatctttcaaagatgacgggaactacaagacgcgtgctgaagtcaagtttgaaggtgatacccttgttaatcgtatc
gagttaaaaggtattgattttaaagaagatggaaacattctcggacacaaactcgagtacaactataactcacacaatgtat
acatcacggcagacaaacaaaagaatggaatcaaagctaacttcaaaattcgccacaacattgaagatggctccgttcaac
tagcagaccattatcaacaaaatactccaattggcgatggccctgtccttttaccagacaaccattacctgtcgacacaatctg
ccctttcgaaagatcccaacgaaaagcgtgaccacatggtccttcttgagtttgtaactgctgctgggattacacatggcatgg
atgagctctacaaataatgaattcgagctggatccagcttcctcgctcactgactcgctgcgctcggtcgttcggctgcggcga
gcggtatcagctcactcaaaggcggtaatacggttatccacagaatcaggggataacgcaggaaagaacatgtgagcaaa
aggccagcaaaaggccaggaaccgtaaaaaggccgcgttgctggcgtttttccataggctccgcccccctgacgagcatcac
aaaaatcgacgctcaagtcagaggtggcgaaacccgacaggactataaagataccaggcgtttccccctggaagctccctcg
tgcgctctcctgttccgaccctgccgcttaccggatacctgtccgcctttctcccttcgggaagcgtggcgctttctcaatgctc
acgctgtaggtatctcagttcggtgtaggtcgttcgctccaagctgggctgtgtgcacgaaccccccgttcagcccgaccgctg
cgccttatccggtaactatcgtcttgagtccaacccggtaagacacgacttatcgccactggcagcagccactggtaacagga
ttagcagagcgaggtatgtaggcggtgctacagagttcttgaagtggtggcctaactacggctacactagaaggacagtattt
ggtatctgcgctctgctgaagccagttaccttcggaaaaagagttggtagctcttgatccggcaaacaaaccaccgctggtag
cggtggtttttttgtttgcaagcagcagattacgcgcagaaaaaaaggatctcaagaagatcctttgatcttttctacggggtct
gacgctcagtggaacgaaaactcacgttaagggattttggtcatgagattatcaaaaaggatcttcacctagatccttttggg
gtgggcgaagaactccagcatgagatccccgcgctggaggatcatccagccggcgtcccggaaaacgattccgaagcccaa
cctttcatagaaggcggcggtggaatcgaaatctcgtgatggcaggttgggcgtcgcttggtcggtcatttcgaaccccagag
tcccgctcagaagaactcgtcaagaaggcgatagaaggcgatgcgctgcgaatcgggagcggcgataccgtaaagcacga
ggaagcggtcagcccattcgccgccaagctcttcagcaatatcacgggtagccaacgctatgtcctgatagcggtccgccac
acccagccggccacagtcgatgaatccagaaaagcggccattttccaccatgatattcggcaagcaggcatcgccatgggtc
acgacgagatcctcgccgtcgggcatccgcgccttgagcctggcgaacagttcggctggcgcgagcccctgatgctcttcgtc
cagatcatcctgatcgacaagaccggcttccatccgagtacgtgctcgctcgatgcgatgtttcgcttggtggtcgaatgggca
ggtagccggatcaagcgtatgcagccgccgcattgcatcagccatgatggatactttctcggcaggagcaaggtgagatgac
aggagatcctgccccggcacttcgcccaatagcagccagtcccttcccgcttcagtgacaacgtcgagcacagctgcgcaag
gaacgcccgtcgtggccagccacgatagccgcgctgcctcgtcttggagttcattcagggcaccggacaggtcggtcttgaca
aaaagaaccgggcgcccctgcgctgacagccggaacacggcggcatcagagcagccgattgtctgttgtgcccagtcatag
ccgaatagcctctccacccaagcggccggagaacctgcgtgcaatccatcttgttcaatcatgcgaaacgatcctcatcctgt
ctcttgatcagatcttgatcccctgcgccatcagatccttggcggcaagaaagccatccagtttactttgcagggcttcccaac
cttaccagagggcgccccagctggcaattccggttcgcttgctgtccataaaaccgcccagtctagctatcgccatgtaagccca
ctgcaagctacctgctttctctttgcgcttgcgttttcccttgtccagatagcccagtagctgacattcatccggggtcagcacc
gtttctgcggactggctttctacgtgttccgcttcctttagcagcccttgcgccctgagtgcttgcggcagcgtgaagctagcgt
ctaccaggacttttacctgtccgacccgttgcaacggaaccccccacggaacccccgcgacacccgctccccaattgcgttaga
acagcggtggattgtcggcttcgttgtgggccttttgagccgcttcctgttctgccgcacgctctttcctcgcccgatagccgag
tcgcttaacggtgtccagatgcagcccgaaatgtttggccgtttgcggccaagagtggccctcgtcgtcgtgataggcgcggat
gcgttcgcggcgtgcagcctgctcggcgagccactcgctgcgttcctgcgccacgagccggacgacgtggcgttcggatagtc
cggtgattcgagcgccttcggcggcggtcacgcgccgctttttgcggacagtcggctgccggttgtagccgtcgctgtagccgt
cgctcatagcaatgcctccatggctgacgcggactttgcgcgccgcgcaactgtgctcgccgccgtgcgcgctgctgcgccctt
ccgcgagatggccgactggcgcgcactgagtgtggcctcgtagaccacgatcccgtccgcccaaatgcgcgacttggttgtg
atccaacgccaaatgctgttggcgatggcgcggacctcgctgtccggtagcggtccgggacacacgtcgttgcacgggaact
cggcgtttcgcgcgtggcactcggcatagatcgcgcggccgagtccgtccacgttccgggtcggcaggtagatccgcatgag
ggcgggacgataggcccacaacctgacggaatcgaacagtgcgcaattccgccctagcggcgtcggagccgctttgtacgtg
gtctgctgacgccagcgcggcggtggcatgttcgcgccgagctcggcctcgatgtggctgagtgtgtagagatctgagtggag
ccattccgtttcccaggcgatgtggccggggtttttggtcatgaggcctgagtaactgcggtcgccgtccacggcgcgccgaa
ggccttcggcgcacgccgccatgtatgcgagcggcttacgccgcgcgtattcggtgcgtggaacaggggcgttgagtgccca
cactgcgtgtgcgtggccgttggcgcgattgcccacgatcgcgttgggcagcggatgggacccccgggcgctgagcgctcgg
agcgctgcgtctggatggtctacgtccacgaccagcaggtttgccagcgctgttgggttcgcctcgatgtaccggcggcctag
ggccgacgcgcggctttggcggtagatcccctcgagcagatcgtcgcttgccagcggccagtacggcagccagagctgctca
aattcgtcggcgacgtggctcacgcttggtagtagaccacgattaatcaccggtgtatggtccgacacgagctccaagtcaga
tatttcgctgaggggccaccccacaactgcacactcccccgctctcccgtcgagccctggtggtggaacaccagcgacagccg
agcacccccaaccacctgtaccaacc
pGZ008 (SEQ ID NO: 9)
actagttgattagctaagcagaaggccatcctgacggatggcctttttgcgtttaatactgtttaaactctagaaatattggatc
gtcggcaccgtcacggccgtgggaggcggcacgatccgcgacgtgatgatcggccgcatccccacggtgctgcgcagtgag
ctctacgccatcccggcgttgatctgtgcgttcgctagcccgtactcctcgtacaggcgcagcgctgcgatcccgaacccggac
agctcggcggccaccgagatgctgtaaacgccacgtaccgagggcaagtcggcatcacgcccggattctgcggccatgtcgc
tccgatcgggaattttctcgcgcaggcccttgtcattgcctactgaagatgctataccaaatctacagcatctgctggagtttac
ctgatggaatgagatggtttcaggaggtggacacgatggctagcaaaggagaagaacttttcactggagttgtcccaattctt
gttgaattagatggtgatgttaatgggcacaaattttctgtcagtggagagggtgaaggtgatgctacatacggaaagcttaccc
ttaaatttatttgcactactggaaaactacctgttccatggccaacacttgtcactactttgacctatggtgttcaatgcttttc
ccgttatccggatcatatgaaacggcatgactttttcaagagtgccatgcccgaaggttatgtacaggaacgcactatatcttt
caaagatgacgggaactacaagacgcgtgctgaagtcaagtttgaaggtgatacccttgttaatcgtatcgagttaaaaggt
attgattttaaagaagatggaaacattctcggacacaaactcgagtacaactataactcacacaatgtatacatcacggcag
acaaacaaaagaatggaatcaaagctaacttcaaaattcgccacaacattgaagatggctccgttcaactagcagaccatta
tcaacaaaatactccaattggcgatggccctgtccttttaccagacaaccattacctgtcgacacaatctgccctttcgaaaga
tcccaacgaaaagcgtgaccacatggtccttcttgagtttgtaactgctgctgggattacacatggcatggatgagctctacaa
ataatgaattcgagctggatccagcttcctcgctcactgactcgctgcgctcggtcgttcggctgcggcgagcggtatcagctc
actcaaaggcggtaatacggttatccacagaatcaggggataacgcaggaaagaacatgtgagcaaaaggccagcaaaa
ggccaggaaccgtaaaaaggccgcgttgctggcgtttttccataggctccgcccccctgacgagcatcacaaaaatcgacgc
tcaagtcagaggtggcgaaacccgacaggactataaagataccaggcgtttccccctggaagctccctcgtgcgctctcctgt
tccgaccctgccgcttaccggatacctgtccgcctttctcccttcgggaagcgtggcgctttctcaatgctcacgctgtaggtat
ctcagttcggtgtaggtcgttcgctccaagctgggctgtgtgcacgaaccccccgttcagcccgaccgctgcgccttatccggt
aactatcgtcttgagtccaacccggtaagacacgacttatcgccactggcagcagccactggtaacaggattagcagagcga
ggtatgtaggcggtgctacagagttcttgaagtggtggcctaactacggctacactagaaggacagtatttggtatctgcgctc
tgctgaagccagttaccttcggaaaaagagttggtagctcttgatccggcaaacaaaccaccgctggtagcggtggtttttttg
tttgcaagcagcagattacgcgcagaaaaaaaggatctcaagaagatcctttgatcttttctacggggtctgacgctcagtgg
aacgaaaactcacgttaagggattttggtcatgagattatcaaaaaggatcttcacctagatccttttggggtgggcgaagaa
ctccagcatgagatccccgcgctggaggatcatccagccggcgtcccggaaaacgattccgaagcccaacctttcatagaag
gcggcggtggaatcgaaatctcgtgatggcaggttgggcgtcgcttggtcggtcatttcgaaccccagagtcccgctcagaag
aactcgtcaagaaggcgatagaaggcgatgcgctgcgaatcgggagcggcgataccgtaaagcacgaggaagcggtcagc
ccattcgccgccaagctcttcagcaatatcacgggtagccaacgctatgtcctgatagcggtccgccacacccagccggccac
agtcgatgaatccagaaaagcggccattttccaccatgatattcggcaagcaggcatcgccatgggtcacgacgagatcctc
gccgtcgggcatccgcgccttgagcctggcgaacagttcggctggcgcgagcccctgatgctcttcgtccagatcatcctgatc
gacaagaccggcttccatccgagtacgtgctcgctcgatgcgatgtttcgcttggtggtcgaatgggcaggtagccggatcaa
gcgtatgcagccgccgcattgcatcagccatgatggatactttctcggcaggagcaaggtgagatgacaggagatcctgccc
cggcacttcgcccaatagcagccagtcccttcccgcttcagtgacaacgtcgagcacagctgcgcaaggaacgcccgtcgtg
gccagccacgatagccgcgctgcctcgtcttggagttcattcagggcaccggacaggtcggtcttgacaaaaagaaccgggc
gcccctgcgctgacagccggaacacggcggcatcagagcagccgattgtctgttgtgcccagtcatagccgaatagcctctcc
acccaagcggccggagaacctgcgtgcaatccatcttgttcaatcatgcgaaacgatcctcatcctgtctcttgatcagatctt
gatcccctgcgccatcagatccttggcggcaagaaagccatccagtttactttgcagggcttcccaaccttaccagagggcgc
cccagctggcaattccggttcgcttgctgtccataaaaccgcccagtctagctatcgccatgtaagcccactgcaagctacctgc
tttctctttgcgcttgcgttttcccttgtccagatagcccagtagctgacattcatccggggtcagcaccgtttctgcggactgg
ctttctacgtgttccgcttcctttagcagcccttgcgccctgagtgcttgcggcagcgtgaagctagcgtctaccaggactttta
cctgtccgacccgttgcaacggaaccccccacggaacccccgcgacacccgctccccaattgcgttagaacagcggtggattg
tcggcttcgttgtgggccttttgagccgcttcctgttctgccgcacgctctttcctcgcccgatagccgagtcgcttaacggtgt
ccagatgcagcccgaaatgtttggccgtttgcggccaagagtggccctcgtcgtcgtgataggcgcggatgcgttcgcggcgtg
cagcctgctcggcgagccactcgctgcgttcctgcgccacgagccggacgacgtggcgttcggatagtccggtgattcgagcg
ccttcggcggcggtcacgcgccgctttttgcggacagtcggctgccggttgtagccgtcgctgtagccgtcgctcatagcaatg
cctccatggctgacgcggactttgcgcgccgcgcaactgtgctcgccgccgtgcgcgctgctgcgcccttccgcgagatggcc
gactggcgcgcactgagtgtggcctcgtagaccacgatcccgtccgcccaaatgcgcgacttggttgtgatccaacgccaaat
gctgttggcgatggcgcggacctcgctgtccggtagcggtccgggacacacgtcgttgcacgggaactcggcgtttcgcgcgt
ggcactcggcatagatcgcgcggccgagtccgtccacgttccgggtcggcaggtagatccgcatgagggcgggacgatagg
cccacaacctgacggaatcgaacagtgcgcaattccgccctagcggcgtcggagccgctttgtacgtggtctgctgacgcca
gcgcggcggtggcatgttcgcgccgagctcggcctcgatgtggctgagtgtgtagagatctgagtggagccattccgtttccc
aggcgatgtggccggggtttttggtcatgaggcctgagtaactgcggtcgccgtccacggcgcgccgaaggccttcggcgca
cgccgccatgtatgcgagcggcttacgccgcgcgtattcggtgcgtggaacaggggcgttgagtgcccacactgcgtgtgcgt
ggccgttggcgcgattgcccacgatcgcgttgggcagcggatgggacccccgggcgctgagcgctcggagcgctgcgtctgg
atggtctacgtccacgaccagcaggtttgccagcgctgttgggttcgcctcgatgtaccggcggcctagggccgacgcgcggc
tttggcggtagatcccctcgagcagatcgtcgcttgccagcggccagtacggcagccagagctgctcaaattcgtcggcgacg
tggctcacgcttggtagtagaccacgattaatcaccggtgtatggtccgacacgagctccaagtcagatatttcgctgagggg
ccaccccacaactgcacactcccccgctctcccgtcgagccctggtggtggaacaccagcgacagccgagcacccccaacca
cctgtaccaacc
pGZ009 (SEQ ID NO: 10)
actagttgattagctaagcagaaggccatcctgacggatggcctttttgcgtttaatactgtttaaactctagaaatattggatc
gtcggcaccgtcacggccgtgggaggcggcacgatccgcgacgtgatgatcggccgcatccccacggtgctgcgcagtgag
ctctacgccatcccggcgttgatctgtgcgttcgctgcgtcaactggtccaggagggcgttccaggcctgccggcgatgcacca
ccccgtgcacgagtacgagggtggggccggagccggcgatgtcgtgaggcaagtccatggacagatgttaccgctatcacg
agtaacatctactgccagccgatagtggggcccgtcgcccacggatgtgggcgacgggccccgacatcgaccccgattcggg
tcggtcgggccgatgcgggaaagatggtaaccggtctgtatggctagcaaaggagaagaacttttcactggagttgtcccaa
ttcttgttgaattagatggtgatgttaatgggcacaaattttctgtcagtggagagggtgaaggtgatgctacatacggaaagct
tacccttaaatttatttgcactactggaaaactacctgttccatggccaacacttgtcactactttgacctatggtgttcaatgc
ttttcccgttatccggatcatatgaaacggcatgactttttcaagagtgccatgcccgaaggttatgtacaggaacgcactata
tctttcaaagatgacgggaactacaagacgcgtgctgaagtcaagtttgaaggtgatacccttgttaatcgtatcgagttaaa
aggtattgattttaaagaagatggaaacattctcggacacaaactcgagtacaactataactcacacaatgtatacatcacg
gcagacaaacaaaagaatggaatcaaagctaacttcaaaattcgccacaacattgaagatggctccgttcaactagcagac
cattatcaacaaaatactccaattggcgatggccctgtccttttaccagacaaccattacctgtcgacacaatctgccctttcga
aagatcccaacgaaaagcgtgaccacatggtccttcttgagtttgtaactgctgctgggattacacatggcatggatgagctc
tacaaataatgaattcgagctggatccagcttcctcgctcactgactcgctgcgctcggtcgttcggctgcggcgagcggtatc
agctcactcaaaggcggtaatacggttatccacagaatcaggggataacgcaggaaagaacatgtgagcaaaaggccagc
aaaaggccaggaaccgtaaaaaggccgcgttgctggcgtttttccataggctccgcccccctgacgagcatcacaaaaatcg
acgctcaagtcagaggtggcgaaacccgacaggactataaagataccaggcgtttccccctggaagctccctcgtgcgctctc
ctgttccgaccctgccgcttaccggatacctgtccgcctttctcccttcgggaagcgtggcgctttctcaatgctcacgctgtag
gtatctcagttcggtgtaggtcgttcgctccaagctgggctgtgtgcacgaaccccccgttcagcccgaccgctgcgccttatcc
ggtaactatcgtcttgagtccaacccggtaagacacgacttatcgccactggcagcagccactggtaacaggattagcagag
cgaggtatgtaggcggtgctacagagttcttgaagtggtggcctaactacggctacactagaaggacagtatttggtatctgc
gctctgctgaagccagttaccttcggaaaaagagttggtagctcttgatccggcaaacaaaccaccgctggtagcggtggttt
ttttgtttgcaagcagcagattacgcgcagaaaaaaaggatctcaagaagatcctttgatcttttctacggggtctgacgctca
gtggaacgaaaactcacgttaagggattttggtcatgagattatcaaaaaggatcttcacctagatccttttggggtgggcga
agaactccagcatgagatccccgcgctggaggatcatccagccggcgtcccggaaaacgattccgaagcccaacctttcata
gaaggcggcggtggaatcgaaatctcgtgatggcaggttgggcgtcgcttggtcggtcatttcgaaccccagagtcccgctca
gaagaactcgtcaagaaggcgatagaaggcgatgcgctgcgaatcgggagcggcgataccgtaaagcacgaggaagcgg
tcagcccattcgccgccaagctcttcagcaatatcacgggtagccaacgctatgtcctgatagcggtccgccacacccagccg
gccacagtcgatgaatccagaaaagcggccattttccaccatgatattcggcaagcaggcatcgccatgggtcacgacgaga
tcctcgccgtcgggcatccgcgccttgagcctggcgaacagttcggctggcgcgagcccctgatgctcttcgtccagatcatcc
tgatcgacaagaccggcttccatccgagtacgtgctcgctcgatgcgatgtttcgcttggtggtcgaatgggcaggtagccgg
atcaagcgtatgcagccgccgcattgcatcagccatgatggatactttctcggcaggagcaaggtgagatgacaggagatcc
tgccccggcacttcgcccaatagcagccagtcccttcccgcttcagtgacaacgtcgagcacagctgcgcaaggaacgcccg
tcgtggccagccacgatagccgcgctgcctcgtcttggagttcattcagggcaccggacaggtcggtcttgacaaaaagaac
cgggcgcccctgcgctgacagccggaacacggcggcatcagagcagccgattgtctgttgtgcccagtcatagccgaatagc
ctctccacccaagcggccggagaacctgcgtgcaatccatcttgttcaatcatgcgaaacgatcctcatcctgtctcttgatca
gatcttgatcccctgcgccatcagatccttggcggcaagaaagccatccagtttactttgcagggcttcccaaccttaccagag
ggcgccccagctggcaattccggttcgcttgctgtccataaaaccgcccagtctagctatcgccatgtaagcccactgcaagcta
cctgctttctctttgcgcttgcgttttcccttgtccagatagcccagtagctgacattcatccggggtcagcaccgtttctgcgg
actggctttctacgtgttccgcttcctttagcagcccttgcgccctgagtgcttgcggcagcgtgaagctagcgtctaccaggac
ttttacctgtccgacccgttgcaacggaaccccccacggaacccccgcgacacccgctccccaattgcgttagaacagcggtg
gattgtcggcttcgttgtgggccttttgagccgcttcctgttctgccgcacgctctttcctcgcccgatagccgagtcgcttaac
ggtgtccagatgcagcccgaaatgtttggccgtttgcggccaagagtggccctcgtcgtcgtgataggcgcggatgcgttcgcg
gcgtgcagcctgctcggcgagccactcgctgcgttcctgcgccacgagccggacgacgtggcgttcggatagtccggtgattc
gagcgccttcggcggcggtcacgcgccgctttttgcggacagtcggctgccggttgtagccgtcgctgtagccgtcgctcatag
caatgcctccatggctgacgcggactttgcgcgccgcgcaactgtgctcgccgccgtgcgcgctgctgcgcccttccgcgaga
tggccgactggcgcgcactgagtgtggcctcgtagaccacgatcccgtccgcccaaatgcgcgacttggttgtgatccaacgc
caaatgctgttggcgatggcgcggacctcgctgtccggtagcggtccgggacacacgtcgttgcacgggaactcggcgtttcg
cgcgtggcactcggcatagatcgcgcggccgagtccgtccacgttccgggtcggcaggtagatccgcatgagggcgggacg
ataggcccacaacctgacggaatcgaacagtgcgcaattccgccctagcggcgtcggagccgctttgtacgtggtctgctgac
gccagcgcggcggtggcatgttcgcgccgagctcggcctcgatgtggctgagtgtgtagagatctgagtggagccattccgtt
tcccaggcgatgtggccggggtttttggtcatgaggcctgagtaactgcggtcgccgtccacggcgcgccgaaggccttcggc
gcacgccgccatgtatgcgagcggcttacgccgcgcgtattcggtgcgtggaacaggggcgttgagtgcccacactgcgtgt
gcgtggccgttggcgcgattgcccacgatcgcgttgggcagcggatgggacccccgggcgctgagcgctcggagcgctgcgt
ctggatggtctacgtccacgaccagcaggtttgccagcgctgttgggttcgcctcgatgtaccggcggcctagggccgacgcg
cggctttggcggtagatcccctcgagcagatcgtcgcttgccagcggccagtacggcagccagagctgctcaaattcgtcggc
gacgtggctcacgcttggtagtagaccacgattaatcaccggtgtatggtccgacacgagctccaagtcagatatttcgctga
ggggccaccccacaactgcacactcccccgctctcccgtcgagccctggtggtggaacaccagcgacagccgagcaccccc
aaccacctgtaccaacc
pYH038 (SEQ ID NO: 11)
gtctgaccacccaccacgggtaacgcggaggggctcggcgagatgcatctcgccgagcccctccgcgtcggttcaggcgcgctt
gcgttccgaactggatgatttcgctgtggccttcttcgccgccgtcttggccggagctttcttcgccggcttcttcttggccggt
gcggccttcttcccggcggcgtcgacgctgcgctgcagggccgccaccagatccaccacttcggcgtcctcgccgctcttctcg
acctcggcgacggggatgaccttcttgccgcccgactcgatcatctcgtcgagcagttcacgcaactgcacctggtagtcgtc
ggtgaattcggtggggtcgaagtcgtcggccatgctgtcgacgagcgtctcggccatcgcgagttccttcgccttcggcttgtc
gacgccgtcgagcgaggagaactcggcgacgcgtacctcgtccggccacagcagcgtctggatcaccagcaccccgtcgcg
tgaccgcatcgcggccagtcgggtgcgctgccggagggtgaagtggacgagggctgtgcggtcgctgttctcgagggcagtc
gcgagaaggacatacgccttcggagagctggaatccggttcgaggaagtaactcttctcgaacagaatgggatcgatctgtt
cggtcggcacgaactgcagaacgggaatctcgtgtttctcggcggcaggcagtttcccgaaatcatcgtccgtcaacaccacg
cgcacgccgtcgtcggagtcgtaggccttgtcgatatcggcgtattggacgacgttgccgcattcggagcagacgcggttgta
tttgatccggccgccgtccttggcatggacctggtgaaacctgatgtcgtgatcctcggttgccgaatagaccttgaccggcac
attcacgagtccgaacgctatcgagcccttccatatcgaccgcatgcgttccatgatggtctccttttcgcgttcgcgcgacagt
gaatagaggtccgctgtgacacaagaatccctgttacttctcgaccgtattgattcggatgattcctacgcgagcctgcggaac
gaccaggaattctgggagccgctggcccgccgagccctggaggagctcgggctgccggtgccgccggtgctgcgggtgccc
ggcgagagcaccaaccccgtactggtcggcgagcccgacccggtgatcaagctgttcggcgagcactggtgcggtccggag
agcctcgcgtcggagtcggaggcgtacgcggtcctggcggacgccccggtgccggtgccccgcctcctcggccgcggcgagc
tgcggcccggcaccggagcctggccgtggccctacctggtgatgagccggatgaccggcaccacctggcggtccgcgatgga
cggcacgaccgaccggaacgcgctgctcgccctggcccgcgaactcggccgggtgctcggccggctgcacagggtgccgct
gaccgggaacaccgtgctcaccccccattccgaggtcttcccggaactgctgcgggaacgccgcgcggcgaccgtcgagga
ccaccgcgggtggggctacctctcgccccggctgctggaccgcctggaggactggctgccggacgtggacacgctgctggcc
ggccgcgaaccccggttcgtccacggcgacctgcacgggaccaacatcttcgtggacctggccgcgaccgaggtcaccggg
atcgtcgacttcaccgacgtctatgcgggagactcccgctacagcctggtgcaactgcatctcaacgccttccggggcgaccg
cgagatcctggccgcgctgctcgacggggcgcagtggaagcggaccgaggacttcgcccgcgaactgctcgccttcaccttc
ctgcacgacttcgaggtgttcgaggagaccccgctggatctctccggcttcaccgatccggaggaactggcgcagttcctctg
ggggccgccggacaccgcccccggcgcctgagtcgcgctcccgtcgactcgccacggccggcccggaccagtgggggcgtt
gaggggagtgctgacgcgtggcccggctaagcgggcgtgggggagcgcggtgcgttggacgagatgcaattgcgcggggc
ggggaacccggatgcgcctgcgaggtcccgaaacatgccggtcgcgcttcccggtccaccaggttgggagtcggtgtactga
gtcgggcccggacggaacatcctagtttcccgaactaccgtggtggcattcactcatcccaccaaccgtcgtcaccgcagcgc
acacctcgcgactgcatcacacccgatcgccaggccgacgacgaagaggccagaggctttctacgagcggatacggccgcg
aacgttagcgggatcactgggggcgtgttccgagtcgccggcggtcgggtaaaattgctggttcgtgtgttctttgcgcagtt
tttctcattcaacgcgagcgtcattttcctaggagtcttgttgatcaccgctaccgacctggaagttcgagctggagtgcggac
gttgctgtccgcgcccgggccggctttgcgggtgcagcccggcgaccgcatcgggctggtcggccgcaacggtgccggcaag
acgaccaccctccgcattctcgcgggggagggggagccgtatgcgggtgccgtggtgcgcaccggcgacctcggatacctcc
cgcaggacccgaaagaaggcgacctcgacgtcctcgcgaaggaccgcgtgttgtccgcgcgcggcctcgacgcgctgctgc
gggacatggagaagcagcagatcctgatgtcggaggtcgtcgacgacgccgagcgcgacaaggcggtgcgcaagtacggc
aatctcgaggaccgcttctcggccctcggggggtacgaggcagagagcgaagccgcacgcatctgcaacagcctcggcctg
gccgaccgcatcctcggccagcagctgcacaccctgtcgggtggtcagcgtcgccgacatcaccgatggggaagatcgggctc
gccacttcgggctcatgatcctagaaatattttatctgattaataagatgatcttcttgagatcgttttggtctgcgcgtaatct
cttgctctgaaaacgaaaaaaccgccttgcagggcggtttttcgaaggttctctgagctaccaactctttgaaccgaggtaact
ggcttggaggagcgcagtcaccaaaacttgtcctttcagtttagccttaaccggcgcatgacttcaagactaactcctctaaat
caattaccagtggctgctgccagtggtgcttttgcatgtctttccgggttggactcaagacgatagttaccggataaggcgcag
cggtcggactgaacggggggttcgtgcatacagtccagcttggagcgaactgcctacccggaactgagtgtcaggcgtggaa
tgagacaaacgcggccataacagcggaatgacaccggtaaaccgaaaggcaggaacaggagagcgcacgagggagccg
ccagggggaaacgcctggtatctttatagtcctgtcgggtttcgccaccactgatttgagcgtcagatttcgtgatgcttgtcag
gggggcggagcctatggaaaaacggctttgccgcggccctctcacttccctgttaagtatcttcctggcatcttccaggaaatc
tccgccccgttcgtaagccatttccgctcgccgcagtcgaacgaccgagcgtagcgagtcagtgagcgaggaagcggaatat
atcctgtatcacatattctgctgacgcaccggtgcagccttttttctcctgccacatgaagcacttcactgacaccctcatcagt
gccaacatagtaagccagtatacactccgctagccatgggcaaatattatacgcaaggcgacaaggtgctgatgccgctggc
gattcaggttcatcatgtaggtgtaaaacgagttagcccttagtgactcgaattctctagtcga
pJD078 (SEQ ID NO: 12)
ggtaatacggttatccacagaatcaggggataacgcaggaaagaacatgtgagcaaaaggccagcaaaaggccaggaac
cgtaaaaaggccgcgttgctggcgtttttccataggctccgcccccctgacgagcatcacaaaaatcgacgctcaagtcaga
ggtggcgaaacccgacaggactataaagataccaggcgtttccccctggaagctccctcgtgcgctctcctgttccgaccctgc
cgcttaccggatacctgtccgcctttctcccttcgggaagcgtggcgctttctcaatgctcacgctgtaggtatctcagttcggt
gtaggtcgttcgctccaagctgggctgtgtgcacgaaccccccgttcagcccgaccgctgcgccttatccggtaactatcgtct
tgagtccaacccggtaagacacgacttatcgccactggcagcagccactggtaacaggattagcagagcgaggtatgtagg
cggtgctacagagttcttgaagtggtggcctaactacggctacactagaaggacagtatttggtatctgcgctctgctgaagcc
agttaccttcggaaaaagagttggtagctcttgatccggcaaacaaaccaccgctggtagcggtggtttttttgtttgcaagca
gcagattacgcgcagaaaaaaaggatctcaagaagatcctttgatcttttctacggggtctgacgctcagtggaacgaaaac
tcacgttaagggattttggtcatgagattatcaaaaaggatcttcacctagatccttttggggtgggcgaagaactccagcatg
agatccccgcgctggaggatcatccagccggcgtcccggaaaacgattccgaagcccaacctttcatagaaggcggcggtg
gaatcgaaatctcgtgatggcaggttgggcgtcgcttggtcggtcatttcgaaccccagagtcccgctcagaagaactcgtca
agaaggcgatagaaggcgatgcgctgcgaatcgggagcggcgataccgtaaagcacgaggaagcggtcagcccattcgcc
gccaagctcttcagcaatatcacgggtagccaacgctatgtcctgatagcggtccgccacacccagccggccacagtcgatg
aatccagaaaagcggccattttccaccatgatattcggcaagcaggcatcgccatgggtcacgacgagatcctcgccgtcgg
gcatccgcgccttgagcctggcgaacagttcggctggcgcgagcccctgatgctcttcgtccagatcatcctgatcgacaaga
ccggcttccatccgagtacgtgctcgctcgatgcgatgtttcgcttggtggtcgaatgggcaggtagccggatcaagcgtatgc
agccgccgcattgcatcagccatgatggatactttctcggcaggagcaaggtgagatgacaggagatcctgccccggcactt
cgcccaatagcagccagtcccttcccgcttcagtgacaacgtcgagcacagctgcgcaaggaacgcccgtcgtggccagcca
cgatagccgcgctgcctcgtcttggagttcattcagggcaccggacaggtcggtcttgacaaaaagaaccgggcgcccctgc
gctgacagccggaacacggcggcatcagagcagccgattgtctgttgtgcccagtcatagccgaatagcctctccacccaag
cggccggagaacctgcgtgcaatccatcttgttcaatcatgcgaaacgatcctcatcctgtctcttgatcagatcttgatcccct
gcgccatcagatccttggcggcaagaaagccatccagtttactttgcagggcttcccaaccttaccagagggcgccccagctg
gcaattccggttcgcttgctgtccataaaaccgcccagtctagctatcgccatgtaagcccactgcaagctacctgctttctctt
tgcgcttgcgttttcccttgtccagatagcccagtagctgacattcatccggggtcagcaccgtttctgcggactggctttctacg
tgttccgcttcctttagcagcccttgcgccctgagtgcttgcggcagcgtgaagctagcgtctaccaggacttttacctgtccga
cccgttgcaacggaaccccccacggaacccccgcgacacccgctccccaattgcgttagaacagcggtggattgtcggcttc
gttgtgggccttttgagccgcttcctgttctgccgcacgctctttcctcgcccgatagccgagtcgcttaacggtgtccagatgc
agcccgaaatgtttggccgtttgcggccaagagtggccctcgtcgtcgtgataggcgcggatgcgttcgcggcgtgcagcctg
ctcggcgagccactcgctgcgttcctgcgccacgagccggacgacgtggcgttcggatagtccggtgattcgagcgccttcgg
cggcggtcacgcgccgctttttgcggacagtcggctgccggttgtagccgtcgctgtagccgtcgctcatagcaatgcctccat
ggctgacgcggactttgcgcgccgcgcaactgtgctcgccgccgtgcgcgctgctgcgcccttccgcgagatggccgactggc
gcgcactgagtgtggcctcgtagaccacgatcccgtccgcccaaatgcgcgacttggttgtgatccaacgccaaatgctgttg
gcgatggcgcggacctcgctgtccggtagcggtccgggacacacgtcgttgcacgggaactcggcgtttcgcgcgtggcact
cggcatagatcgcgcggccgagtccgtccacgttccgggtcggcaggtagatccgcatgagggcgggacgataggcccaca
acctgacggaatcgaacagtgcgcaattccgccctagcggcgtcggagccgctttgtacgtggtctgctgacgccagcgcgg
cggtggcatgttcgcgccgagctcggcctcgatgtggctgagtgtgtagagatctgagtggagccattccgtttcccaggcgat
gtggccggggtttttggtcatgaggcctgagtaactgcggtcgccgtccacggcgcgccgaaggccttcggcgcacgccgcc
atgtatgcgagcggcttacgccgcgcgtattcggtgcgtggaacaggggcgttgagtgcccacactgcgtgtgcgtggccgtt
ggcgcgattgcccacgatcgcgttgggcagcggatgggacccccgggcgctgagcgctcggagcgctgcgtctggatggtct
acgtccacgaccagcaggtttgccagcgctgttgggttcgcctcgatgtaccggcggcctagggccgacgcgcggctttggcg
gtagatcccctcgagcagatcgtcgcttgccagcggccagtacggcagccagagctgctcaaattcgtcggcgacgtggctc
acgcttggtagtagaccacgattaatcaccggtgtatggtccgacacgagctccaagtcagatatttcgctgaggggccaccc
cacaactgcacactcccccgctctcccgtcgagccctggtggtggaacaccagcgacagccgagcacccccaaccacctgta
ccaaccactagttgattcgttaccaattatgacaacttgacggctacatcattcactttttcttcacaaccggcacggaactcgc
tcgggctggccccggtgcattttttaaatacccgcgagaaatagagttgatcgtcaaaaccaacattgcgaccgacggtggcg
ataggcatccgggtggtgctcaaaagcagcttcgcctggctgatacgttggtcctcgcgccagcttaagacgctaatccctaa
ctgctggcggaaaagatgtgacagacgcgacggcgacaagcaaacatgctgtgcgacgctggcgatatcaaaattgctgtc
tgccaggtgatcgctgatgtactgacaagcctcgcgtacccgattatccatcggtggatggagcgactcgttaatcgcttccat
gcgccgcagtaacaattgctcaagcagatttatcgccagcagctccgaatagcgcccttccccttgcccggcgttaatgatttg
cccaaacaggtcgctgaaatgcggctggtgcgcttcatccgggcgaaagaaccccgtattggcaaatattgacggccagtta
agccattcatgccagtaggcgcgcggacgaaagtaaacccactggtgataccattcgcgagcctccggatgacgaccgtagt
gatgaatctctcctggcgggaacagcaaaatatcacccggtcggcaaacaaattctcgtccctgatttttcaccaccccctgac
cgcgaatggtgagattgagaatataacctttcattcccagcggtcggtcgataaaaaaatcgagataaccgttggcctcaatc
ggcgttaaacccgccaccagatgggcattaaacgagtatcccggcagcaggggatcattttgcgcttcagccatacttttcat
actcccgccattcagagaagaaaccaattgtccatattgcatcagacattgccgtcactgcgtcttttactggctcttctcgcta
accaaaccggtaaccccgcttattaaaagcattctgtaacaaagcgggaccaaagccatgacaaaaacgcgtaacaaaagt
gtctataatcacggcagaaaagtccacattgattatttgcacggcgtcacactttgctatgccatagcatttttatccataagat
tagcggatcctacctgacgctttttatcgcaactctctactgtttctccatacagcggataaagtagcaaagagaaggaggtta
ggaatgacggtctgcgcaaaaaaacacgttcatctcactcgcgatgctgcggagcagttactggctgatattgatcgacgcct
tgatcagttattgcccgtggagggagaacgggatgttgtgggtgccgcgatgcgtgaaggtgcgctggcaccgggaaaacgt
attcgccccatgttgctgttgctgaccgcccgcgatctgggttgcgctgtcagccatgacggattactggatttggcctgtgcgg
tggaaatggtccacgcggcttcgctgatccttgacgatatgccctgcatggacgatgcgaagctgcggcgcggacgccctacc
attcattctcattacggagagcatgtggcaatactggcggcggttgccttgctgagtaaagcctttggcgtaattgccgatgca
gatggcctcacgccgctggcaaaaaatcgggcggtttctgaactgtcaaacgccatcggcatgcaaggattggttcagggtc
agttcaaggatctgtctgaaggggataagccgcgcagcgctgaagctattttgatgacgaatcactttaaaaccagcacgct
gttttgtgcctccatgcagatggcctcgattgttgcgaatgcctccagcgaagcgcgtgattgcctgcatcgtttttcacttgatc
ttggtcaggcatttcaactgctggacgatttgaccgatggcatgaccgacaccggtaaggatagcaatcaggacgccggtaa
atcgacgctggtcaatctgttaggcccgagggcggttgaagaacgtctgagacaacatcttcagcttgccagtgagcatctct
ctgcggcctgccaacacgggcacgccactcaacattttattcaggcctggtttgacaaaaaactcgctgccgtcagttaataat
actagagctcaaggaggtactagatgaataatccgtcgttactcaatcatgcggtcgaaacgatggcagttggctcgaaaag
ttttgcgacagcctcaaagttatttgatgcaaaaacccggcgcagcgtactgatgctctacgcctggtgccgccattgtgacga
tgttattgacgatcagacgctgggctttcaggcccggcagcctgccttacaaacgcccgaacaacgtctgatgcaacttgaga
tgaaaacgcgccaggcctatgcaggatcgcagatgcacgaaccggcgtttgcggcttttcaggaagtggctatggctcatga
tatcgccccggcttacgcgtttgatcatctggaaggcttcgccatggatgtacgcgaagcgcaatacagccaactggatgata
cgctgcgctattgctatcacgttgcaggcgttgtcggcttgatgatggcgcaaatcatgggcgtgcgggataacgccacgctg
gaccgcgcctgtgaccttgggctggcatttcagttgaccaatattgctcgcgatattgtggacgatgcgcatgcgggccgctgt
tatctgccggcaagctggctggagcatgaaggtctgaacaaagagaattatgcggcacctgaaaaccgtcaggcgctgagc
cgtatcgcccgtcgtttggtgcaggaagcagaaccttactatttgtctgccacagccggcctggcagggttgcccctgcgttcc
gcctgggcaatcgctacggcgaagcaggtttaccggaaaataggtgtcaaagttgaacaggccggtcagcaagcctgggat
cagcggcagtcaacgaccacgcccgaaaaattaacgctgctgctggccgcctctggtcaggcccttacttcccggatgcggg
ctcatcctccccgccctgcgcatctctggcagcgcccgctctaataatactagagctcaaggaggtactagatgaaaccaact
acggtaattggtgcaggcttcggtggcctggcactggcaattcgtctacaagctgcggggatccccgtcttactgcttgaacaa
cgtgataaacccggcggtcgggcttatgtctacgaggatcaggggtttacctttgatgcaggcccgacggttatcaccgatcc
cagtgccattgaagaactgtttgcactggcaggaaaacagttaaaagagtatgtcgaactgctgccggttacgccgttttacc
gcctgtgttgggagtcagggaaggtctttaattacgataacgatcaaacccggctcgaagcgcagattcagcagtttaatccc
cgcgatgtcgaaggttatcgtcagtttctggactattcacgcgcggtgtttaaagaaggctatctaaagctcggtactgtccctt
ttttatcgttcagagacatgcttcgcgccgcacctcaactggcgaaactgcaagcatggagaagcgtttacagtaaggttgcc
agttacatcgaagatgaacatctgcgccaggcgttttctttccactcgctgttggtgggcggcaatcccttcgccacctcatcca
tttatacgttgatacacgcgctggagcgtgagtggggcgtctggtttccgcgtggcggcaccggcgcattagttcaggggatg
ataaagctgtttcaggatctgggtggcgaagtcgtgttaaacgccagagtcagccatatggaaacgacaggaaacaagatt
gaagccgtgcatttagaggacggtcgcaggttcctgacgcaagccgtcgcgtcaaatgcagatgtggttcatacctatcgcga
cctgttaagccagcaccctgccgcggttaagcagtccaacaaactgcaaactaagcgcatgagtaactctctgtttgtgctcta
ttttggtttgaatcaccatcatgatcagctcgcgcatcacacggtttgtttcggcccgcgttaccgcgagctgattgacgaaatt
tttaatcatgatggcctcgcagaggacttctcactttatctgcacgcgccctgtgtcacggattcgtcactggcgcctgaaggtt
gcggcagttactatgtgttggcgccggtgccgcatttaggcaccgcgaacctcgactggacggttgaggggccaaaactacg
cgaccgtatttttgcgtaccttgagcagcattacatgcctggcttacggagtcagctggtcacgcaccggatgtttacgccgttt
gattttcgcgaccagcttaatgcctatcatggctcagccttttctgtggagcccgttcttacccagagcgcctggtttcggccgc
ataaccgcgataaaaccattactaatctctacctggtcggcgcaggcacgcatcccggcgcaggcattcctggcgtcatcggc
tcggcaaaagcgacagcaggtttgatgctggaggatctgatataaggtctggtgtcaaaaataatactagagccaggcatca
aataaaacgaaaggctcagtcgaaagactgggcctttcgttttatctgttgtttgtcggtgaacgctctctactagagccaggc
atcaaataaaacgaaaggctcagtcgaaagactgggcctttcgttttatctgttgtttgtcggtgaacgctctctactagtagc
ggccgctgcagtccggcaaaaaagggcaaggtgtcaccaccctgccctttttctttaaaaccgaaaagattacttcgcgttat
gcaggcttcctcgctcactgactcgctgcgctcggtcgttcggctgcggcgagcggtatcagctcactcaaaggcggtaatac
ggttatccacagaatcagg
pJD095 (SEQ ID NO: 13)
gatagtgagttatgcaaccaaatgacgtggctttgttgaataaatcgaacttttgctgagttgaaggatcagctcgatataaac
gcagaaaggcccacccgaaggtgagccagtgtgactctagtagagagcgttcaccgacaaacaacagataaaacgaaagg
cccagtctttcgactgagcctttcgttttatttgatgcctggttatcatgagaccggtcttattccccgaacagggaatccagcca
tccaacaacagtggtttcaatgccgtcggcgtcgaggccataatcggcgagcacttcattgcgggacgcgtgatccaggtact
tctggggcacggcgatttgtcggcgaggggtatccacctcagaggcgttaagcgcatcagagagcaaggatcccacgccgc
cgtggatgacgccgtcttcgatggtgatcacgaggtcatgatcatcagacagcgcgaccaaggactgcgggatggggacaat
ccagcgggggtcaacaaccgtgacgttcacgccgtgctgtttaatcctggaagcaacgtcaagtgcaacagttgcgcgctcg
cctaccgcaatgatgagaactgatggcgcatcgtcggttgattcaacgtcagtggcgtcttcatatgcgaggacatccacgcc
gtcttccaaggtgtcgatagcaacaattggagttggcaagtcgcccttggggaaacgcacaactgtggggccatcatcgatg
gaaatagcctcattgagcagctcacgcaaggaatcctcatcacgtggtgccgccacctgcacgcctggaacgatcgaggtca
gcgccatatcccagacgccattgtggctcgctccatccgaacccgtgacacctgagcgatcaagcaccaaagtaacaggctg
gttgagcatgcccacatccatgagcagctgatcaaaagcgcggttcaagaacgtggagtaaatagccaccacagggtgtttt
ccacccaatgcgaggcctgcggcagaagttaccgcgtgctgctcagcaatgccgacatcaaagaatcggttggggaaattgg
cttcgaacttggacagaccggtaggacctgccatcgcggcggtgatggcaacaacgttttcattctgcgcaccaatcttgacc
agctcatcgctgaacacagaggtccaaccgggctttgatgcagatttaggagctcctgtgagcggatcgatgacgcccgtgg
agtgcatcaattcgtccaaatcctgctcagcaggcgcgtaaccacgacccttttcggtgaccatgtgcacgatgatggggcca
tcataatcatgagcgtatttcagcgcattgtcgacagctttttggttatgtccatcaaccggacccacgtatttcatgcccagttc
agggaacatttcggtgggaatgacggtgctcttcacaccttctttaaatgcatggagcgcttcaaaagtacgctcccctaccca
ccccatggatttcagggacgtcttgcccttttccatgaagcgatcatagaaaggctgcatgcgaaggcccgcaaggttttccg
caaatccgccaatggttggagaataactccggccattgtcattgactacgacaacaactttgcggtctttaccagcagcaata
ttgttcagtgcttcccaacacatgccgccagttagagcgccatcaccaacgacagcaaccacactatgcgtggtatcgccatc
caactgcttggctttagacaaaccatccgcataagacaaggccgccgaagcatgcgaagactcagtccaatcgtgctcacttt
cagcacggcaggtgtaaccagaaaggccatctttttgacgcaaagaatcaaaatctttagcgcgacccgtcaggatcttatg
cacataggactggtgagaagtatcaaagatgatcgggtcttgaggcgaatcgaaaactcgatgaagaccgatggttaattcc
actacgcccaaatttggacctaagtggccaccagttgctgcgactttatcgaccaggaaagttcggatttctttggcaagagc
gtccaaatcctcatcattaagggcctttaagtcagcaggtgttgaaatactgttcagaattcccatgcttttccttctctttctaa
ccaccggattcggcggacagcacatacagtccgccgggccggttccgaccgctgtacttctagatttccccctcaggaaaagc
agagcaccgcgttcgcaacatcgatcctgcgaacgttcactctttcaagcctacgaacacagaagctgcgaccagttgtagtc
aaccatgaactccgcgaacgcaacagtcgaaatccgaacaattcacacaaaaagttgatcttgtagcgaacaggcacgaaa
cagcggattggcgcgggggtcacgacaccccgggcgacagccccggaaaaggactcgttatcatcaaatgcgcaggtcgtg
gcggtgtggaagctactcgcccgtaccctacgacagcccggagacatcactcgaggtgaagacgaaagggcctcgtgatac
gcctatttttataggttaatgtcatgataataatggtttcttagacgtcgatatctggcgaaaatggttaatcaagggcactctc
gactgtcttctttatcatgcaactcgtaggacaggtgccacttcatccggggtcagcaccaccggcaagcgccgcgacggccg
aggtcttccgatctcctgaagccagggcagatccgtgcacagcaccttgccgtagaagaacagcaaggccgccaatgcctga
cgatgcgtggagaccgaaaccttgcgctcgttcgccagccaggacagaaatgcctcgacttcgctgctgcccaaggttgccg
ggtgacgcacaccgtggaaacggatgaaggcacgaacccagttgacataagcctgttcggttcgtaaactgtaatgcaagta
gcgtatgcgctcacgcaactggtccagaaccttgaccgaacgcagcggtggtaacggcgcagtggcggttttcatggcttgtt
atgactgtttttttgtacagtctatgcctcgggcatccaagcagcaagcgcgttacgccgtgggtcgatgtttgatgttatggag
cagcaacgatgttacgcagcagggcagtcgccctaaaacaaagttaggtggctcaagtatgggcatcattcgcacatgtagg
ctcggccctgaccaagtcaaatccatgcgggctgctcttgatcttttcggtcgtgagttcggagacgtagccacctactcccaa
catcagccggactccgattacctcgggaacttgctccgtagtaagacattcatcgcgcttgctgccttcgaccaagaagcggtt
gttggcgctctcgcggcttacgttctgcccaagtttgagcagccgcgtagtgagatctatatctatgatctcgcagtctccggcg
agcaccggaggcagggcattgccaccgcgctcatcaatctcctcaagcatgaggccaacgcgcttggtgcttatgtgatctac
gtgcaagcagattacggtgacgatcccgcagtggctctctatacaaagttgggcatacgggaagaagtgatgcactttgatat
cgacccaagtaccgccacctaacaattcgttcaagccgagatcggcttcccggccgcggcttggtttcatcagccatccgcttg
ccctcatctgttacgccggcggtagccggccagcctcgcagagcaggattcccgttgagcaccgccaggtgcgaataaggga
cagtgaagaaggaacacccgctcgcgggtgggcctacttcacctatcctgcccggctgacgccgttggatacaccaaggaaa
gtctacacgaaccctttggcaaaatcctgtatatcgtgcgaaaaaggatggatataccgaaaaaatcgctataatgaccccg
aagcagggttatgcagcggaaaagcgctgcttccctgctgttttgtgggccgcaaagcgctccggcttaactcaggcgttagt
cccatcacccgctttatgcgcgcagccacttatttcagcgctccgccctgttccaaaggctttctagccttttgggcctccagcg
cttgtgcagcaagcgccaccagctatccttcttgcagcgctgcgccgctttcgtggtccagcggtttttcttgggctgcgcccttc
ttcaaggccgggcgctcccttttggccttcgggtctaactctgcggtcaagcggacccgcattctgcgggccgcttaccttggcc
gttagacatcatggtaaatctgcgcagacagccctgtgcagctgaaacgcggttacgtatagcttgccatatgtctagccata
cgtaaccgcaggtaaaaggcatatttttcgcgtgtcatggctagtaaataacaccggtgtcatttagagtcagggaaagaca
atgaaaaacgaagaaagccaccgggcggcaacccgatgactttcgcttatcacccagcacacacctgggagaaatcacggt
catgagtttacagactcatgcgcagaatgcgcacactaaaacacctacccgcgtcgagcgcgaccgtggtggactggacaa
caccccagcatctgccagtgaccgcgaccttttacgcgatcatctaggccgcgatgtactccacggttcagtcacacgagactt
taaaaaggcctatcgacgcaacgctgacggcacgaactcgccgcgtatgtatcgcttcgagactgatgctttaggacggtgc
gagtacgccatgctcaccaccaagcagtacgccgccgtcctggtcgtagacgttgaccaagtaggtaccgcaggcggtgacc
ccgcagacttaaacccgtacgtccgcgacgtggtgcgctcactgattactcatagcgtcgggccagcctggggggtattaac
ccaactaacggcaaagcccagttcatatggcttattgaccctgtctacgctgaccgtaacggtaaatctgcgcagatgaagct
tcttgcagcaaccacgcgtgtgctgggtgagcttttagaccatgacccgcacttttcccaccgctttagccgcaacccgttctac
acaggcaaagcccctaccgcttatcgttggtataggcagcacaaccgggtgatgcgccttggagacttgataaagcaggtaa
gggatatggcaggacacgaccagttcaaccccaccccacgccagcaattcagctctggccgcgaacttatcaacgcggtcaa
gacccgccgtgaagaagcccaagcattcaaagcactcgcccaggacgtagacgcggaaatcgccggtggtctcgaccagta
tgacccggaacttatcgacggtgtgcgtgtgctctggattgtccaaggaaccgcagcacgcgacgaaacagcctttagacat
gcgcttaagactggccaccgcttgcgccagcaaggccaacgcctgacagacgcagcaatcatcgacgcctatgagcacgcc
tacaacgtcgcacacacccacggcggtgcaggccgcgacaacgagatgccacccatgcgcgaccgccaaaccatggcaag
gcgcgtgcgcgggtatgtcgcccaatccaagagcgagacctacagcggctctaacgcaccaggtaaagccaccagcagcga
gcggaaagccttggccacgatgggacgcagaggcggacaaaaagccgcacaacgctggaaaacagaccccgagggcaaa
tatgcgcaagcacaaaggtcgaagcttgaaaagacgcaccgtaagaaaaaggctcaaggacgatctacgaagtcccgtatt
agccaaatggtgaacgatcagtatttccagacagggacagttcccacgtgggctgaaataggggcagaggtaggagtctctc
gcgccacggttgctaggcatgtcgcggagctaaagaagagcggtgactatccggacgtttaaggggtctcataccgtaagca
atatacggttcccctgccgttaggcagttagataaaacctcacttgaagaaaaccttgaggggcagggcagcttatatgcttc
aaagcatgacttcctctgttctcctagacctcgcaaccctccgccataacctcaccgaattgcttaagtttcacccctttgacgg
cttaaccgccgcataggaaatgccctccggactaattgtctggagggcatttttgctacctgctgtgcactttgtacataaggc
aacaagattttggcaaaaggtggaagggcgaattgctgcgcgtaaccaccacacccgccgcgcttaatgcgccgctacagg
gcgcgtccattcgccattcaggctgcgcaactgttgggaagggcgatcggtgcgggcctcttcgctattacgccagctggcga
aagggggatgtgctgcaaggcgattaagttgggtaacg
pJD096 (SEQ ID NO: 14)
gatagtgagttatgcaaccaaatgacgtggctttgttgaataaatcgaacttttgctgagttgaaggatcagctcgatataaac
gcagaaaggcccacccgaaggtgagccagtgtgactctagtagagagcgttcaccgacaaacaacagataaaacgaaagg
cccagtctttcgactgagcctttcgttttatttgatgcctggttatcatgagaccggtcttactctgcgtcaaacgcttccaggat
ggcttggcgcagctcaggggcgctaagctgatccactagccatggagaaaacacaaatggtgtggcatcaacagcgtcgaa
aagcttctgcggttccgcccactcgaactcctccacctcatcatccagtggctccacaaattcccccaccgctaaacgcacgag
gtggaccgggcacaactcccactccacaatgccggacgcgtcgacagcacggtactggtaatcaggcagaatctcttgaata
tccaagaaagaatctacctccagccccaactcatcgacacccctgcgacgaatcgcatccgcgtttgtctcatccggaccagg
gtgcccacacatagagttcgtccacacaccaggccatgtcttcttcgacaatgcacgacgcgtcaccaacagctccccacgcg
ggttcaaaatataggtggaaaacgcgaaatgcagaggcgtgtccttagtgtgcaccgtagctttcggcgcagtaccaatagg
attgccctcggaatcagctaaaacaaccagttcaacctcagtagtcatgcccctaagcttagacatgcttttccttctctttcta
accaccggattcggcggacagcacatacagtccgccgggccggttccgaccgctgtacttctagatttccccctcaggaaaag
cagagcaccgcgttcgcaacatcgatcctgcgaacgttcactctttcaagcctacgaacacagaagctgcgaccagttgtagt
caaccatgaactccgcgaacgcaacagtcgaaatccgaacaattcacacaaaaagttgatcttgtagcgaacaggcacgaa
acagcggattggcgcgggggtcacgacaccccgggcgacagccccggaaaaggactcgttatcatcaaatgcgcaggtcgt
ggcggtgtggaagctactcgcccgtaccctacgacagcccggagacatcactcgaggtgaagacgaaagggcctcgtgata
cgcctatttttataggttaatgtcatgataataatggtttcttagacgtcgatatctggcgaaaatggttaatcaagggcactct
cgactgtcttctttatcatgcaactcgtaggacaggtgccacttcatccggggtcagcaccaccggcaagcgccgcgacggcc
gaggtcttccgatctcctgaagccagggcagatccgtgcacagcaccttgccgtagaagaacagcaaggccgccaatgcctg
acgatgcgtggagaccgaaaccttgcgctcgttcgccagccaggacagaaatgcctcgacttcgctgctgcccaaggttgcc
gggtgacgcacaccgtggaaacggatgaaggcacgaacccagttgacataagcctgttcggttcgtaaactgtaatgcaagt
agcgtatgcgctcacgcaactggtccagaaccttgaccgaacgcagcggtggtaacggcgcagtggcggttttcatggcttgt
tatgactgtttttttgtacagtctatgcctcgggcatccaagcagcaagcgcgttacgccgtgggtcgatgtttgatgttatgga
gcagcaacgatgttacgcagcagggcagtcgccctaaaacaaagttaggtggctcaagtatgggcatcattcgcacatgtag
gctcggccctgaccaagtcaaatccatgcgggctgctcttgatcttttcggtcgtgagttcggagacgtagccacctactccca
acatcagccggactccgattacctcgggaacttgctccgtagtaagacattcatcgcgcttgctgccttcgaccaagaagcgg
ttgttggcgctctcgcggcttacgttctgcccaagtttgagcagccgcgtagtgagatctatatctatgatctcgcagtctccgg
cgagcaccggaggcagggcattgccaccgcgctcatcaatctcctcaagcatgaggccaacgcgcttggtgcttatgtgatct
acgtgcaagcagattacggtgacgatcccgcagtggctctctatacaaagttgggcatacgggaagaagtgatgcactttga
tatcgacccaagtaccgccacctaacaattcgttcaagccgagatcggcttcccggccgcggcttggtttcatcagccatccgc
ttgccctcatctgttacgccggcggtagccggccagcctcgcagagcaggattcccgttgagcaccgccaggtgcgaataag
ggacagtgaagaaggaacacccgctcgcggggggcctacttcacctatcctgcccggctgacgccgttggatacaccaagg
aaagtctacacgaaccctttggcaaaatcctgtatatcgtgcgaaaaaggatggatataccgaaaaaatcgctataatgacc
ccgaagcagggttatgcagcggaaaagcgctgcttccctgctgttttgtgggccgcaaagcgctccggcttaactcaggcgtt
agtcccatcacccgctttatgcgcgcagccacttatttcagcgctccgccctgttccaaaggctttctagccttttgggcctccag
cgcttgtgcagcaagcgccaccagctatccttcttgcagcgctgcgccgctttcgtggtccagcggtttttcttgggctgcgccc
ttcttcaaggccgggcgctcccttttggccttcgggtctaactctgcggtcaagcggacccgcattctgcgggccgcttaccttg
gccgttagacatcatggtaaatctgcgcagacagccctgtgcagctgaaacgcggttacgtatagcttgccatatgtctagcc
atacgtaaccgcaggtaaaaggcatatttttcgcgtgtcatggctagtaaataacaccggtgtcatttagagtcagggaaaga
caatgaaaaacgaagaaagccaccgggcggcaacccgatgactttcgcttatcacccagcacacacctgggagaaatcacg
gtcatgagtttacagactcatgcgcagaatgcgcacactaaaacacctacccgcgtcgagcgcgaccgtggtggactggaca
acaccccagcatctgccagtgaccgcgaccttttacgcgatcatctaggccgcgatgtactccacggttcagtcacacgagac
tttaaaaaggcctatcgacgcaacgctgacggcacgaactcgccgcgtatgtatcgcttcgagactgatgctttaggacggtg
cgagtacgccatgctcaccaccaagcagtacgccgccgtcctggtcgtagacgttgaccaagtaggtaccgcaggcggtgac
cccgcagacttaaacccgtacgtccgcgacgtggtgcgctcactgattactcatagcgtcgggccagcctgggtgggtattaa
cccaactaacggcaaagcccagttcatatggcttattgaccctgtctacgctgaccgtaacggtaaatctgcgcagatgaagc
ttcttgcagcaaccacgcgtgtgctgggtgagcttttagaccatgacccgcacttttcccaccgctttagccgcaacccgttcta
cacaggcaaagcccctaccgcttatcgttggtataggcagcacaaccgggtgatgcgccttggagacttgataaagcaggta
agggatatggcaggacacgaccagttcaaccccaccccacgccagcaattcagctctggccgcgaacttatcaacgcggtca
agacccgccgtgaagaagcccaagcattcaaagcactcgcccaggacgtagacgcggaaatcgccggtggtctcgaccagt
atgacccggaacttatcgacggtgtgcgtgtgctctggattgtccaaggaaccgcagcacgcgacgaaacagcctttagaca
tgcgcttaagactggccaccgcttgcgccagcaaggccaacgcctgacagacgcagcaatcatcgacgcctatgagcacgc
ctacaacgtcgcacacacccacggcggtgcaggccgcgacaacgagatgccacccatgcgcgaccgccaaaccatggcaa
ggcgcgtgcgcgggtatgtcgcccaatccaagagcgagacctacagcggctctaacgcaccaggtaaagccaccagcagcg
agcggaaagccttggccacgatgggacgcagaggcggacaaaaagccgcacaacgctggaaaacagaccccgagggcaa
atatgcgcaagcacaaaggtcgaagcttgaaaagacgcaccgtaagaaaaaggctcaaggacgatctacgaagtcccgta
ttagccaaatggtgaacgatcagtatttccagacagggacagttcccacgtgggctgaaataggggcagaggtaggagtctc
tcgcgccacggttgctaggcatgtcgcggagctaaagaagagcggtgactatccggacgtttaaggggtctcataccgtaag
caatatacggttcccctgccgttaggcagttagataaaacctcacttgaagaaaaccttgaggggcagggcagcttatatgct
tcaaagcatgacttcctctgttctcctagacctcgcaaccctccgccataacctcaccgaattgcttaagtttcacccctttgac
ggcttaaccgccgcataggaaatgccctccggactaattgtctggagggcatttttgctacctgctgtgcactttgtacataag
gcaacaagattttggcaaaaggtggaagggcgaattgctgcgcgtaaccaccacacccgccgcgcttaatgcgccgctaca
gggcgcgtccattcgccattcaggctgcgcaactgttgggaagggcgatcggtgcgggcctcttcgctattacgccagctggc
gaaagggggatgtgctgcaaggcgattaagttgggtaacg
pJD097 (SEQ ID NO: 15)
gatagtgagttatgcaaccaaatgacgtggctttgttgaataaatcgaacttttgctgagttgaaggatcagctcgatataaac
gcagaaaggcccacccgaaggtgagccagtgtgactctagtagagagcgttcaccgacaaacaacagataaaacgaaagg
cccagtctttcgactgagcctttcgttttatttgatgcctggttatcatgagaccggtcttactctgcgtcaaacgcttccaggat
ggcttggcgcagctcaggggcgctaagctgatccactagccatggagaaaacacaaatggtgtggcatcaacagcgtcgaa
aagcttctgcggttccgcccactcgaactcctccacctcatcatccagtggctccacaaattcccccaccgctaaacgcacgag
gtggaccgggcacaactcccactccacaatgccggacgcgtcgacagcacggtactggtaatcaggcagaatctcttgaata
tccaagaaagaatctacctccagccccaactcatcgacacccctgcgacgaatcgcatccgcgtttgtctcatccggaccagg
gtgcccacacatagagttcgtccacacaccaggccatgtcttcttcgacaatgcacgacgcgtcaccaacagctccccacgcg
ggttcaaaatataggtggaaaacgcgaaatgcagaggcgtgtccttagtgtgcaccgtagctttcggcgcagtaccaatagg
attgccctcggaatcagctaaaacaaccagttcaacctcagtagtcatgcccctaagcttagacatATACGTCCCTCCCT
TCTCAAATTTAGTTCttattccccgaacagggaatccagccatccaacaacagtggtttcaatgccgtcggcgtcgagg
ccataatcggcgagcacttcattgcgggacgcgtgatccaggtacttctggggcacggcgatttgtcggcgaggggtatccac
ctcagaggcgttaagcgcatcagagagcaaggatcccacgccgccgtggatgacgccgtcttcgatggtgatcacgaggtca
tgatcatcagacagcgcgaccaaggactgcgggatggggacaatccagcgggggtcaacaaccgtgacgttcacgccgtgc
tgtttaatcctggaagcaacgtcaagtgcaacagttgcgcgctcgcctaccgcaatgatgagaactgatggcgcatcgtcggt
tgattcaacgtcagtggcgtcttcatatgcgaggacatccacgccgtcttccaaggtgtcgatagcaacaattggagttggca
agtcgcccttggggaaacgcacaactgtggggccatcatcgatggaaatagcctcattgagcagctcacgcaaggaatcctc
atcacgtggtgccgccacctgcacgcctggaacgatcgaggtcagcgccatatcccagacgccattgtggctcgctccatccg
aacccgtgacacctgagcgatcaagcaccaaagtaacaggctggttgagcatgcccacatccatgagcagctgatcaaaag
cgcggttcaagaacgtggagtaaatagccaccacagggtgttttccacccaatgcgaggcctgcggcagaagttaccgcgtg
ctgctcagcaatgccgacatcaaagaatcggttggggaaattggcttcgaacttggacagaccggtaggacctgccatcgcg
gcggtgatggcaacaacgttttcattctgcgcaccaatcttgaccagctcatcgctgaacacagaggtccaaccgggctttga
tgcagatttaggagctcctgtgagcggatcgatgacgcccgtggagtgcatcaattcgtccaaatcctgctcagcaggcgcgt
aaccacgacccttttcggtgaccatgtgcacgatgatggggccatcataatcatgagcgtatttcagcgcattgtcgacagctt
tttggttatgtccatcaaccggacccacgtatttcatgcccagttcagggaacatttcggtgggaatgacggtgctcttcacacc
ttctttaaatgcatggagcgcttcaaaagtacgctcccctacccaccccatggatttcagggacgtcttgcccttttccatgaag
cgatcatagaaaggctgcatgcgaaggcccgcaaggttttccgcaaatccgccaatggttggagaataactccggccattgt
cattgactacgacaacaactttgcggtctttaccagcagcaatattgttcagtgcttcccaacacatgccgccagttagagcgc
catcaccaacgacagcaaccacactatgcgtggtatcgccatccaactgcttggctttagacaaaccatccgcataagacaa
ggccgccgaagcatgcgaagactcagtccaatcgtgctcactttcagcacggcaggtgtaaccagaaaggccatctttttgac
gcaaagaatcaaaatctttagcgcgacccgtcaggatcttatgcacataggactggtgagaagtatcaaagatgatcgggtc
ttgaggcgaatcgaaaactcgatgaagaccgatggttaattccactacgcccaaatttggacctaagtggccaccagttgctg
cgactttatcgaccaggaaagttcggatttctttggcaagagcgtccaaatcctcatcattaagggcctttaagtcagcaggtg
ttgaaatactgttcagaattcccatgcttttccttctctttctaaccaccggattcggcggacagcacatacagtccgccgggcc
ggttccgaccgctgtacttctagatttccccctcaggaaaagcagagcaccgcgttcgcaacatcgatcctgcgaacgttcact
ctttcaagcctacgaacacagaagctgcgaccagttgtagtcaaccatgaactccgcgaacgcaacagtcgaaatccgaac
aattcacacaaaaagttgatcttgtagcgaacaggcacgaaacagcggattggcgcgggggtcacgacaccccgggcgac
agccccggaaaaggactcgttatcatcaaatgcgcaggtcgtggcggtgtggaagctactcgcccgtaccctacgacagccc
ggagacatcactcgaggtgaagacgaaagggcctcgtgatacgcctatttttataggttaatgtcatgataataatggtttctt
agacgtcgatatctggcgaaaatggttaatcaagggcactctcgactgtcttctttatcatgcaactcgtaggacaggtgccac
ttcatccggggtcagcaccaccggcaagcgccgcgacggccgaggtcttccgatctcctgaagccagggcagatccgtgcac
agcaccttgccgtagaagaacagcaaggccgccaatgcctgacgatgcgtggagaccgaaaccttgcgctcgttcgccagc
caggacagaaatgcctcgacttcgctgctgcccaaggttgccgggtgacgcacaccgtggaaacggatgaaggcacgaacc
cagttgacataagcctgttcggttcgtaaactgtaatgcaagtagcgtatgcgctcacgcaactggtccagaaccttgaccga
acgcagcggtggtaacggcgcagtggcggttttcatggcttgttatgactgtttttttgtacagtctatgcctcgggcatccaag
cagcaagcgcgttacgccgtgggtcgatgtttgatgttatggagcagcaacgatgttacgcagcagggcagtcgccctaaaa
caaagttaggtggctcaagtatgggcatcattcgcacatgtaggctcggccctgaccaagtcaaatccatgcgggctgctctt
gatcttttcggtcgtgagttcggagacgtagccacctactcccaacatcagccggactccgattacctcgggaacttgctccgt
agtaagacattcatcgcgcttgctgccttcgaccaagaagcggttgttggcgctctcgcggcttacgttctgcccaagtttgag
cagccgcgtagtgagatctatatctatgatctcgcagtctccggcgagcaccggaggcagggcattgccaccgcgctcatcaa
tctcctcaagcatgaggccaacgcgcttggtgcttatgtgatctacgtgcaagcagattacggtgacgatcccgcagtggctct
ctatacaaagttgggcatacgggaagaagtgatgcactttgatatcgacccaagtaccgccacctaacaattcgttcaagcc
gagatcggcttcccggccgcggcttggtttcatcagccatccgcttgccctcatctgttacgccggcggtagccggccagcctc
gcagagcaggattcccgttgagcaccgccaggtgcgaataagggacagtgaagaaggaacacccgctcgcgggtgggcct
acttcacctatcctgcccggctgacgccgttggatacaccaaggaaagtctacacgaaccctttggcaaaatcctgtatatcgt
gcgaaaaaggatggatataccgaaaaaatcgctataatgaccccgaagcagggttatgcagcggaaaagcgctgcttccct
gctgttttgtgggccgcaaagcgctccggcttaactcaggcgttagtcccatcacccgctttatgcgcgcagccacttatttcag
cgctccgccctgttccaaaggctttctagccttttgggcctccagcgcttgtgcagcaagcgccaccagctatccttcttgcagc
gctgcgccgctttcgtggtccagcggtttttcttgggctgcgcccttcttcaaggccgggcgctcccttttggccttcgggtctaa
ctctgcggtcaagcggacccgcattctgcgggccgcttaccttggccgttagacatcatggtaaatctgcgcagacagccctgt
gcagctgaaacgcggttacgtatagcttgccatatgtctagccatacgtaaccgcaggtaaaaggcatatttttcgcgtgtcat
ggctagtaaataacaccggtgtcatttagagtcagggaaagacaatgaaaaacgaagaaagccaccgggcggcaacccga
tgactttcgcttatcacccagcacacacctgggagaaatcacggtcatgagtttacagactcatgcgcagaatgcgcacacta
aaacacctacccgcgtcgagcgcgaccgtggtggactggacaacaccccagcatctgccagtgaccgcgaccttttacgcga
tcatctaggccgcgatgtactccacggttcagtcacacgagactttaaaaaggcctatcgacgcaacgctgacggcacgaac
tcgccgcgtatgtatcgcttcgagactgatgctttaggacggtgcgagtacgccatgctcaccaccaagcagtacgccgccgt
cctggtcgtagacgttgaccaagtaggtaccgcaggcggtgaccccgcagacttaaacccgtacgtccgcgacgtggtgcgc
tcactgattactcatagcgtcgggccagcctgggtgggtattaacccaactaacggcaaagcccagttcatatggcttattgac
cctgtctacgctgaccgtaacggtaaatctgcgcagatgaagcttcttgcagcaaccacgcgtgtgctgggtgagcttttagac
catgacccgcacttttcccaccgctttagccgcaacccgttctacacaggcaaagcccctaccgcttatcgttggtataggcag
cacaaccgggtgatgcgccttggagacttgataaagcaggtaagggatatggcaggacacgaccagttcaaccccacccca
cgccagcaattcagctctggccgcgaacttatcaacgcggtcaagacccgccgtgaagaagcccaagcattcaaagcactcg
cccaggacgtagacgcggaaatcgccggtggtctcgaccagtatgacccggaacttatcgacggtgtgcgtgtgctctggatt
gtccaaggaaccgcagcacgcgacgaaacagcctttagacatgcgcttaagactggccaccgcttgcgccagcaaggccaa
cgcctgacagacgcagcaatcatcgacgcctatgagcacgcctacaacgtcgcacacacccacggcggtgcaggccgcgac
aacgagatgccacccatgcgcgaccgccaaaccatggcaaggcgcgtgcgcgggtatgtcgcccaatccaagagcgagac
ctacagcggctctaacgcaccaggtaaagccaccagcagcgagcggaaagccttggccacgatgggacgcagaggcggac
aaaaagccgcacaacgctggaaaacagaccccgagggcaaatatgcgcaagcacaaaggtcgaagcttgaaaagacgca
ccgtaagaaaaaggctcaaggacgatctacgaagtcccgtattagccaaatggtgaacgatcagtatttccagacagggac
agttcccacgtgggctgaaataggggcagaggtaggagtctctcgcgccacggttgctaggcatgtcgcggagctaaagaa
gagcggtgactatccggacgtttaaggggtctcataccgtaagcaatatacggttcccctgccgttaggcagttagataaaac
ctcacttgaagaaaaccttgaggggcagggcagcttatatgcttcaaagcatgacttcctctgttctcctagacctcgcaaccc
tccgccataacctcaccgaattgcttaagtttcacccctttgacggcttaaccgccgcataggaaatgccctccggactaattg
tctggagggcatttttgctacctgctgtgcactttgtacataaggcaacaagattttggcaaaaggtggaagggcgaattgctg
cgcgtaaccaccacacccgccgcgcttaatgcgccgctacagggcgcgtccattcgccattcaggctgcgcaactgttgggaa
gggcgatcggtgcgggcctcttcgctattacgccagctggcgaaagggggatgtgctgcaaggcgattaagttgggtaacg

### Strain Construction.

RPET competent cells were made using the previously-described method⁴⁸. Briefly, a single colony was selected and inoculated into 10 mL TSB medium and incubated at 30° C. and 250 rpm overnight. The seed culture was then inoculated into 100 mL TSB medium containing 8.5 g/L glycine and 10 g/L sucrose. Cells were harvested until optical density at 600 nm (OD₆₀₀) reached to 0.4-0.5 and washed twice with sterile deionized water. Cells were re-suspended in 10% glycerol and aliquots of 100 μL were frozen at −80° C. for transformation.

For the transformation of replicating plasmid, 50 L competent cells were transformed with approximately 500 ng plasmid DNA by electroporation using a 0.2-cm gap cuvette (at 2500 mV, time constant 5-6 ms). After electric shock, cells were recovered in 600 μL TSB media and incubated at 30° C. and 250 rpm for 4 hrs. Cells were then spread on TSB plates with antibiotics; plates were incubated at 30° C. for two days, or until colonies were visible.

Chromosome modifications in RPET were engineered by transformation of the recipient strains with suicide vectors. These plasmids carried two homologous arms corresponding precisely to each side of the chromosomal target. 2 μg suicide plasmid DNA was transformed via electroporation, as above. Cells were recovered at 30° C. for 12 hrs, and then plated on TSB agar plates with antibiotics. Colonies appeared after 3 days' incubation, and then genotypes were confirmed by colony PCR. A brief description of the strains used in this study is provided in TABLE 3.

TABLE 3
Strains used in this study. N/A, not applicable.

| Strain | | | | Plasmid |
|---|---|---|---|---|
| name | Genus | Species | Genotype | Contained |
| YH_RJ_021 | Rhodococcus | jostii Strain | ΔCrtL-b::HygR | N/A |
| (Strain S3) | | PET | | |
| sJD_R_000 | Rhodococcus | jostii Strain | WT | N/A |
| | | PET | | |
| sJD_R_069 | Rhodococcus | jostii Strain | WT | pJD078 |
| (Strain S1) | | PET | | |
| sJD_R_077 | Rhodococcus | jostii Strain | ΔCrtL-b::HygR | pJD096 |
| (Strain S5) | | PET | | |
| sJD_R_078 | Rhodococcus | jostii Strain | ΔCrtL-b::HygR | pJD095 |
| (Strain S4) | | PET | | |
| sJD_R_078 | Rhodococcus | jostii Strain | ΔCrtL-b::HygR | pJD097 |
| (Strain S6) | | PET | | |
| sJD_R_079 | Rhodococcus | jostii Strain | ΔCrtL-b::HygR | pJD078 and |
| (Strain S7) | | PET | | pJD097 |
| sJD_R_080 | Rhodococcus | jostii Strain | ΔCrtL-b::HygR | pJD078 |
| (Strain S2) | | PET | | |

### Cell Growth and Fluorescence Measurements.

The optical density at 600 nm (OD₆₀₀) was measured in VWR semimicro polystyrene cuvettes, using a Tecan Infinite M200 Pro plate reader (Tecan, Switzerland). The cell growth and fluorescence were measured in black 96-well plates with clear bottom (Greiner Bio-one, Austria), using a Tecan Infinite M200 Pro plate reader. For the measurements of cell growth, Abs₆₀₀ values were converted into OD₆₀₀ values by this experimentally determined relationship:

OD₆₀₀=1.75×Abs₆₀₀.

For the measurements of fluorescence, the excitation and emission wavelengths were 488 and 530 nm, respectively⁴⁸. All the fluorescence values were normalized to cell density (Abs₆₀₀).

### PET Depolymerization Via Alkaline Hydrolysis.

PET was depolymerized as previously described, with modifications⁵⁰. Briefly, the PET decomposition reaction was carried out in a Parr Stirred Reactor (Series 4560 Mini Reactors, 100-600 mL) equipped with a 300 mL PTFE liner (Parr Instrument Company, Moline, IL). 6 g PET powder was dissolved into 60 mL 2 M NaOH. The homogenous PET suspension was then fed into the PTFE vessel, and incubated at 180° C. for 2 hrs with agitation. The reaction mixture was cooled down on ice, and centrifuged at 3500 relative centrifugal force (rcf) to remove the unreacted PET solids. The clear supernatant was neutralized to pH 7.0 with HCl. The resulting mix was transferred to the microbial cultures.

### Quantification of Chemicals.

For the quantification of TPA, a previously-established high-performance liquid chromatography (HPLC) method was applied⁵¹. HPLC analysis was achieved using an Agilent 1260 Infinity II HPLC system (Agilent Technologies, USA) equipped with the Agilent Poroshell 120 EC-C18 column (4.6×100 mm, 2.7 μm) and a UV detector (at 280 nm). The mobile phase consisted of water (0.1% formic acid) and acetonitrile (0.1% formic acid), and a gradient elution was used: 92/8 at 0 min, 74/26 at 5 min, 50/50 at 8 min, and 92/8 at 10 min. The working temperature was set at 60° C., and the flow rate was 1 mL/min. TPA concentration was determined by comparing UV absorbance values to a standard calibration curve with an R² coefficient of 0.995 or higher.

EG was quantified using an Agilent 1260 Infinity II HPLC system equipped with a refractive index detector and an Aminex HPX-87H ion exclusion column (300 mm×7.8 mm, particle size 9 μm; Bio-Rad, USA). The column was held at 40° C., and the samples were isocratically eluted using 0.01 N H₂SO₄ at a flow rate of 1.0 mL/min. EG concentration was determined based on the retention time and the standard calibration curve.

For the extraction and quantification of carotenoids, 5 mL cells were centrifuged (at 3500 rcf for 8 min) and washed twice with deionized H₂O then lyophilized. The lyophilized cell pellet was then subjected to carotenoid extraction, using a previously described method with modifications²⁶. Briefly, the cell pellet was resuspended in 500 μL of a methanol:acetone mixture (7:3) with 0.05% butylated hydroxytoluene (BHT), and then subjected to homogenization using the optional ZR BashingBead Lysis Tubes (0.1 mm beads; Zymo Research, USA). After centrifugation, the carotenoid-containing organic phase was aliquoted to a 1.5 mL tube. This extraction cycle was repeated until all visible color had been removed from the cell pellet. The organic phase containing carotenoids was combined and filtered through a 0.22 μm syringe filter; the resultant suspension was then subjected to HPLC analysis, using an established method⁵². Briefly, the HPLC system quipped with the Agilent Poroshell 120 EC-C18 column (4.6×100 mm, 2.7 μm) and a UV detector (at 474 nm)—was used for a 15-min isocratic elution, with column held at 40° C. and the samples isocratically eluted using methanol/acetonitrile (7:3) at a flow rate of 1 mL/min. The lycopene peak was identified via comparison to commercially-available standards.

### Statistical Analysis and Experimental Replicates.

All experiments were conducted with three independent biological replicates to ensure reproducibility. Unless noted specifically, error bars indicate standard deviation from the mean of these replicates. P-values were calculated using independent student's t tests, unless otherwise specified; P<0.05 was considered significant.

### REFERENCES

1. Filho, W. L. et al. An assessment of attitudes towards plastics and bioplastics in Europe. Sci Total Environ 755, 142732 (2021).
2. Tournier, V. et al. An engineered PET depolymerase to break down and recycle plastic bottles. Nature 580, 216-+(2020).
3. Ragaert, K., Delva, L. & Van Geem, K. Mechanical and chemical recycling of solid plastic waste. Waste Manag 69, 24-58 (2017).
4. Vora, N. et al. Leveling the cost and carbon footprint of circular polymers that are chemically recycled to monomer. Sci Adv 7 (2021).
5. Geyer, R., Jambeck, J. R. & Law, K. L. Production, use, and fate of all plastics ever made. Sci Adv 3 (2017).
6. Rahimi, A. & Garcia, J. M. Chemical recycling of waste plastics for new materials production. Nat Rev Chem 1 (2017).
7. Garcia, J. M. et al. Recyclable, strong thermosets and organogels via paraformaldehyde condensation with diamines. Science 344, 732-735 (2014).
8. Rorrer, N. A. et al. Combining reclaimed PET with bio-based monomers enables plastics upcycling. Joule 3, 1006-1027 (2019).
9. Zhou, L. et al. Alcoholysis of polyethylene terephthalate to produce dioctyl terephthalate using choline chloride-based deep eutectic solvents as efficient catalysts. Green Chem 21, 897-906 (2019).
10. Uekert, T., Kasap, H. & Reisner, E. Photoreforming of Nonrecyclable Plastic Waste over a Carbon Nitride/Nickel Phosphide Catalyst. J Am Chem Soc 141, 15201-15210 (2019).
11. Uekert, T., Kuehnel, M. F., Wakerley, D. W. & Reisner, E. Plastic waste as a feedstock for solar-driven H 2 generation. Energy & Environmental Science 11, 2853-2857 (2018).
12. Zhou, H. et al. Electrocatalytic upcycling of polyethylene terephthalate to commodity chemicals and H2 fuel. Nat Commun 12, 4679 (2021).
13. Yoshida, S. et al. A bacterium that degrades and assimilates poly(ethylene terephthalate). Science 351, 1196-1199 (2016).
14. Wierckx, N. et al. Plastic waste as a novel substrate for industrial biotechnology. Microb Biotechnol 8, 900-903 (2015).
15. Wei, R. et al. Biocatalytic Degradation Efficiency of Postconsumer Polyethylene Terephthalate Packaging Determined by Their Polymer Microstructures. Adv Sci (Weinh) 6, 1900491 (2019).
16. Tiso, T. et al. Towards bio-upcycling of polyethylene terephthalate. Metab Eng 66, 167-178 (2021).
17. Sadler, J. C. & Wallace, S. Microbial synthesis of vanillin from waste poly(ethylene terephthalate). Green Chem 23, 4665-4672 (2021).
18. Werner, A. Z. et al. Tandem chemical deconstruction and biological upcycling of poly(ethylene terephthalate) to beta-ketoadipic acid by Pseudomonas putida KT2440. Metab Eng 67, 250-261 (2021).
19. Karayannidis, G. P., Chatziavgoustis, A. P. & Achilias, D. S. Poly(ethylene terephthalate) recycling and recovery of pure terephthalic acid by alkaline hydrolysis. Adv Polym Tech 21, 250-259 (2002).
20. Cappelletti, M. et al. Biotechnology of Rhodococcus for the production of valuable compounds. Appl Microbiol Biotechnol 104, 8567-8594 (2020).
21. Chen, Z. & Wan, C. Microbial Conversion of Lignin-Based Compounds into Carotenoids by Rhodococci. Appl Biochem Biotechnol 193, 3442-3453 (2021).
22. Kirsh, V. A. et al. A prospective study of lycopene and tomato product intake and risk of prostate cancer. Cancer Epidemiol Biomarkers Prev 15, 92-98 (2006).
23. Javanmardi, J. & Kubota, C. Variation of lycopene, antioxidant activity, total soluble solids and weight loss of tomato during postharvest storage. Postharvest biology and technology 41, 151-155 (2006).
24. Jhou, B. Y., Song, T. Y., Lee, I., Hu, M. L. & Yang, N.C. Lycopene Inhibits Metastasis of Human Liver Adenocarcinoma SK-Hep-1 Cells by Downregulation of NADPH Oxidase 4 Protein Expression. J Agric Food Chem 65, 6893-6903 (2017).
25. Henke, N. A., Heider, S. A., Peters-Wendisch, P. & Wendisch, V. F. Production of the Marine Carotenoid Astaxanthin by Metabolically Engineered Corynebacterium glutamicum. Mar Drugs 14 (2016).
26. Heider, S. A., Peters-Wendisch, P. & Wendisch, V. F. Carotenoid biosynthesis and overproduction in Corynebacterium glutamicum. BMC Microbiol 12, 198 (2012).
27. Kim, S. W. & Keasling, J. D. Metabolic engineering of the nonmevalonate isopentenyl diphosphate synthesis pathway in Escherichia coli enhances lycopene production. Biotechnol Bioeng 72, 408-415 (2001).
28. Harker, M. & Bramley, P. M. Expression of prokaryotic 1-deoxy-D-xylulose-5-phosphatases in Escherichia coli increases carotenoid and ubiquinone biosynthesis. Febs Lett 448, 115-119 (1999).
29. DeLorenzo, D. M., Rottinghaus, A. G., Henson, W. R. & Moon, T. S. Molecular Toolkit for Gene Expression Control and Genome Modification in Rhodococcus opacus PD630. ACS Synth Biol 7, 727-738 (2018).
30. Xiao, Y., Zhao, Z. K. & Liu, P. Mechanistic Studies of IspH in the Deoxyxylulose Phosphate Pathway: Heterolytic C—O Bond Cleavage at C4 Position. Journal of the American Chemical Society 130, 2164-2165 (2008).
31. Heider, S. A., Wolf, N., Hofemeier, A., Peters-Wendisch, P. & Wendisch, V. F. Optimization of the IPP Precursor Supply for the Production of Lycopene, Decaprenoxanthin and Astaxanthin by Corynebacterium glutamicum. Front Bioeng Biotechnol 2, 28 (2014).
32. Hachicho, N., Birnbaum, A. & Heipieper, H. J. Osmotic stress in colony and planktonic cells of Pseudomonas putida mt-2 revealed significant differences in adaptive response mechanisms. AMB Express 7, 62 (2017).
33. Sasoh, M. et al. Characterization of the terephthalate degradation genes of Comamonas sp strain E6. Appl Environ Microb 72, 1825-1832 (2006).
34. Narancic, T. et al. Genome analysis of the metabolically versatile Pseudomonas umsongensis GO16: the genetic basis for PET monomer upcycling into polyhydroxyalkanoates. Microb Biotechnol 14, 2463-2480 (2021).
35. Hara, H., Eltis, L. D., Davies, J. E. & Mohn, W. W. Transcriptomic analysis reveals a bifurcated terephthalate degradation pathway in Rhodococcus sp. strain RHA1. J Bacteriol 189, 1641-1647 (2007).
36. Anthony, W. E. et al. Development of Rhodococcus opacus as a chassis for lignin valorization and bioproduction of high-value compounds. Biotechnol Biofuels 12 (2019).
37. Crawford, R. L., Bromley, J. W. & Perkins-Olson, P. E. Catabolism of protocatechuate by Bacillus macerans. Appl Environ Microbiol 37, 614-618 (1979).
38. Harwood, C. S. & Parales, R. E. The beta-ketoadipate pathway and the biology of self-identity. Annual Review of Microbiology 50, 553-590 (1996).
39. Kersten, P. J., Dagley, S., Whittaker, J. W., Arciero, D. M. & Lipscomb, J. D. 2-pyrone-4,6-dicarboxylic acid, a catabolite of gallic acids in Pseudomonas species. J Bacteriol 152, 1154-1162 (1982).
40. Spence, E. M. et al. The Hydroxyquinol Degradation Pathway in Rhodococcus jostii RHA1 and Agrobacterium Species Is an Alternative Pathway for Degradation of Protocatechuic Acid and Lignin Fragments. Appl Environ Microbiol 86 (2020).
41. Fincher, E. L. & Payne, W. J. Bacterial utilization of ether glycols. Appl Microbiol 10, 542-547 (1962).
42. Muckschel, B. et al. Ethylene glycol metabolism by Pseudomonas putida. Appl Environ Microbiol 78, 8531-8539 (2012).
43. Tiso, T. et al. The metabolic potential of plastics as biotechnological carbon sources-Review and targets for the future. Metabolic Engineering (2021).
44. Heider, S. A., Peters-Wendisch, P., Wendisch, V. F., Beekwilder, J. & Brautaset, T. Metabolic engineering for the microbial production of carotenoids and related products with a focus on the rare C50 carotenoids. Appl Microbiol Biotechnol 98, 4355-4368 (2014).
45. Martin, V. J., Pitera, D. J., Withers, S. T., Newman, J. D. & Keasling, J. D. Engineering a mevalonate pathway in Escherichia coli for production of terpenoids. Nat Biotechnol 21, 796-802 (2003).
46. Rodriguez-Villalon, A., Perez-Gil, J. & Rodriguez-Concepcion, M. Carotenoid accumulation in bacteria with enhanced supply of isoprenoid precursors by upregulation of exogenous or endogenous pathways. J Biotechnol 135, 78-84 (2008).
47. Ugduler, S., Van Geem, K. M., Roosen, M., Delbeke, E. I. P. & De Meester, S. Challenges and opportunities of solvent-based additive extraction methods for plastic recycling. Waste Manag 104, 148-182 (2020).
48. DeLorenzo, D. M., Henson, W. R. & Moon, T. S. Development of Chemical and Metabolite Sensors for Rhodococcus opacus PD630. ACS Synth Biol 6, 1973-1978 (2017).
49. Gibson, D. G. et al. Enzymatic assembly of DNA molecules up to several hundred kilobases. Nat Methods 6, 343-U341 (2009).
50. Singh, S. et al. Recycling of Waste Poly(ethylene terephthalate) Bottles by Alkaline Hydrolysis and Recovery of Pure Nanospindle-Shaped Terephthalic Acid. J Nanosci Nanotechnol 18, 5804-5809 (2018).
51. DeLorenzo, D. M., Diao, J., Carr, R., Hu, Y. & Moon, T. S. An Improved CRISPR Interference Tool to Engineer Rhodococcus opacus. ACS Synth Biol 10, 786-798 (2021).
52. Arvayo-Enríquez, H., Mondaca-Fernández, I., Gortárez-Moroyoqui, P., López-Cervantes, J. & Rodríguez-Ramírez, R. Carotenoids extraction and quantification: a review. Analytical Methods 5, 2916-2924 (2013).

### Example 2: Developing a Novel Microbial Host for Upcycling Waste Polyethylene Terephthalate

This example describes engineered microbes that take wastes as inputs and generate value-added products as outputs.

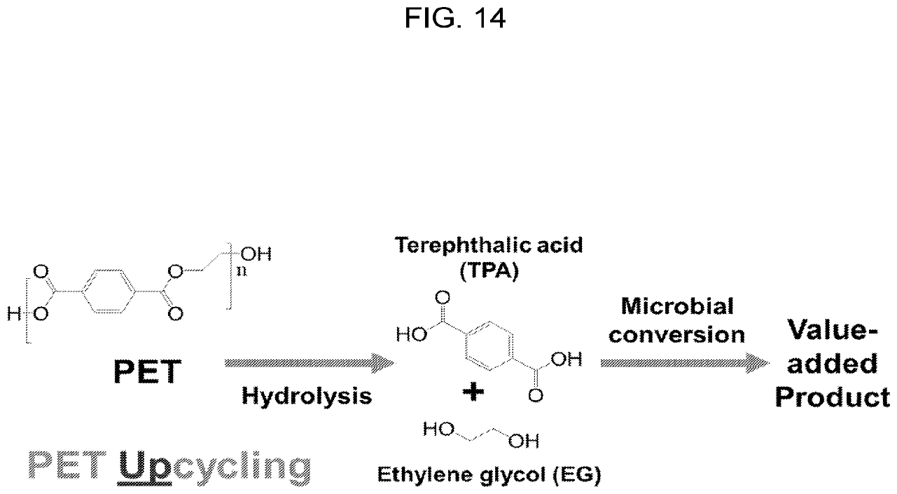

This process is a hybrid process consisting of a hydrolysis step to generate TPA and EG monomers, and microbial conversion of these into value-added product (see e.g., FIG. 14).

We are interested in waste PET upcycling because Waste PET represents 8% of global solid waste and the current PET chemical recycling suffers from the relatively high cost for hydrolysis, monomer purification, and its re-polymerization compared to the virgin PET price.

In contrast, the presently disclosed process uses a novel microbe that is flexible to potentially produce any desirable products, allowing us to cope with market price fluctuations.

Here is demonstrated two higher-value products as a proof-of-concept.

| Conventional technology | This technology |
|---|---|
| 1] engineered leaf-branch | 1] chose aqueous NaOH without adding |
| compost cutinase (LCC) to | any additional catalyst |
| create a variant ICCG | 2] up to 90% depolymerization at 90- |
| 2] used purified ICCG at | 180° C. for 1.5-2 h |
| 72° C. to obtain ~90% | 3] Grew microbes on alkaline hydrolysis |
| depolymerization in 9.3 h | products of PET as the sole carbon |
| 3] re-polymerized the | source without any purification step to |
| purified monomers into PET | produce value-added chemicals |
| Tournier et al. Nature | |
| 580, 216-9 (2020) | |

One problem the present technology solves is efficient PET hydrolysis. Current technology uses a purified leaf and branch compost cutinase (LCC) enzyme variant to achieve ˜90% hydrolysis in 9 h at 72° C. and then make PET again from the purified monomers.

In contrast, this technology use NaOH hydrolysis without adding any additional catalyst to achieve similar yield at higher temp (e.g., ˜90-180° C.) for shorter amount of time and then applies the use of the presently disclosed microbe to produce value-added chemicals w/o any purification step.

The second challenge this technology solves is efficient monomer bioconversion. The main biological issue is that there have been few reports of microbes capable of growing on both TPA and EG. In addition, 2 mol NaOH per 1 mol released TPA is required to maintain pH 8, so the resultant media would have high osmolarity, limiting high TPA/EG loading.

Current technology demonstrated PET-to-PHA biopolymer conversion using purified LCC to degrade PET into TPA and EG at 70° C. for 168 h and then an evolved Pseudomonas strain to produce PHA.

In contrast, the presently disclosed technology required neither purification nor sterilization of the NaOH hydrolysis products. Importantly, the presently disclosed strain used one-order of magnitude higher monomer concentration despite high salt levels due to the use of [NaOH] & HCl for neutralization.

Here is demonstrated efficient monomer bioconversion. Challenges overcome include the identification of a microbial strains capable of growing on both TPA and EG; 2 mol NaOH/1 mol released TPA required to maintain pH 8; and sensitivity to high osmolarity limits high TPA/EG loading.

| Conventional | This technology |
|---|---|
| 1] used purified LCC to | 1] used aqueous NaOH |
| degrade PET into TPA and | without any additional |
| EG at 70° C. for 168 h | catalyst at 150° C. for 1.5 h |
| 2] fed TPA/EG to an | 2] Required neither purification nor |
| evolved Pseudomonas | sterilization; our novel strain |
| strain to produce | grew well on the |
| polyhydroxyalkanoate | NaOH hydrolysis products |
| (PHA) | 3] Grew well on TPA and EG up |
| 3] growth data on TPA | to 0.3M each, total 0.6M |
| and EG only up to 0.03M | despite high [NaOH] |
| Tiso et al. bioRxiv (2020) | & HCl (used for neutralization) |

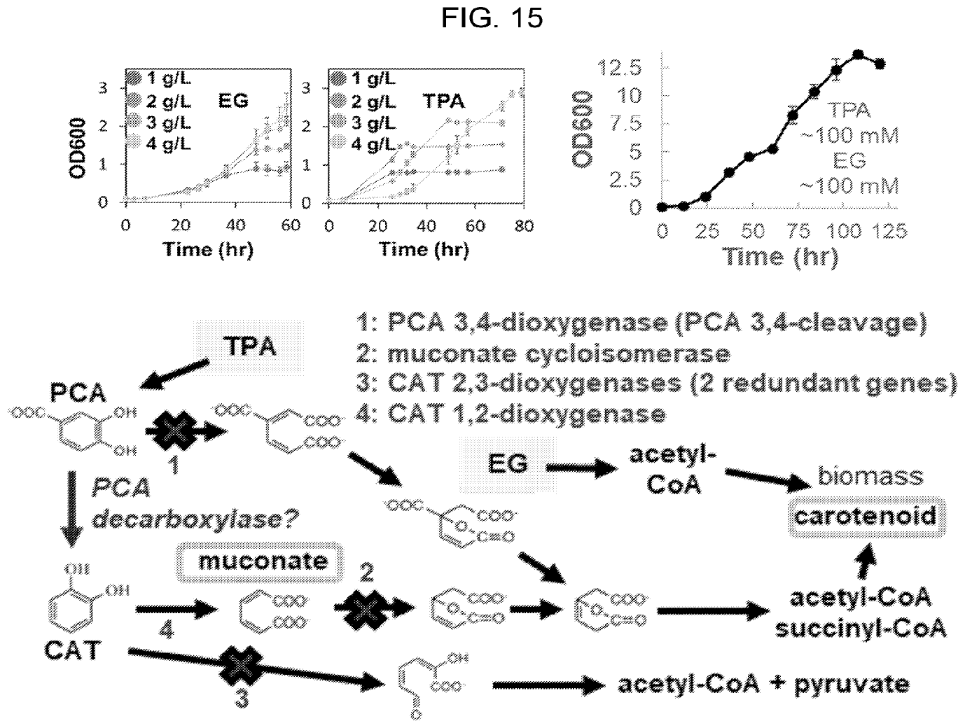

Here is disclosed a novel high salt-tolerant Rhodococcus strain named RPET. The RPET strain was identified by screening many wild-type and mutant bacteria, including Pseudomonas and Rhodococcus and resulted in up to 37% biomass yield per used PET, utilizing both monomers as sole carbon sources (see e.g., FIG. 15). The most reliable measurement method is still by grams of dry cell weight (DCW) or optical density of a sample measured at a wavelength of 600 nm (OD₆₀₀).

Genetic tools were developed for a related species Rhodococcus opacus (see e.g., DeLorenzo et al. An Improved CRISPR Interference Tool to Engineer Rhodococcus opacus ACS Synth. Biol. 2021, 10, 786-798). Fortunately, these tools are functional with some genetic modifications needed to optimize the microbial chassis (minor changes in DNA sequence) in RPET.

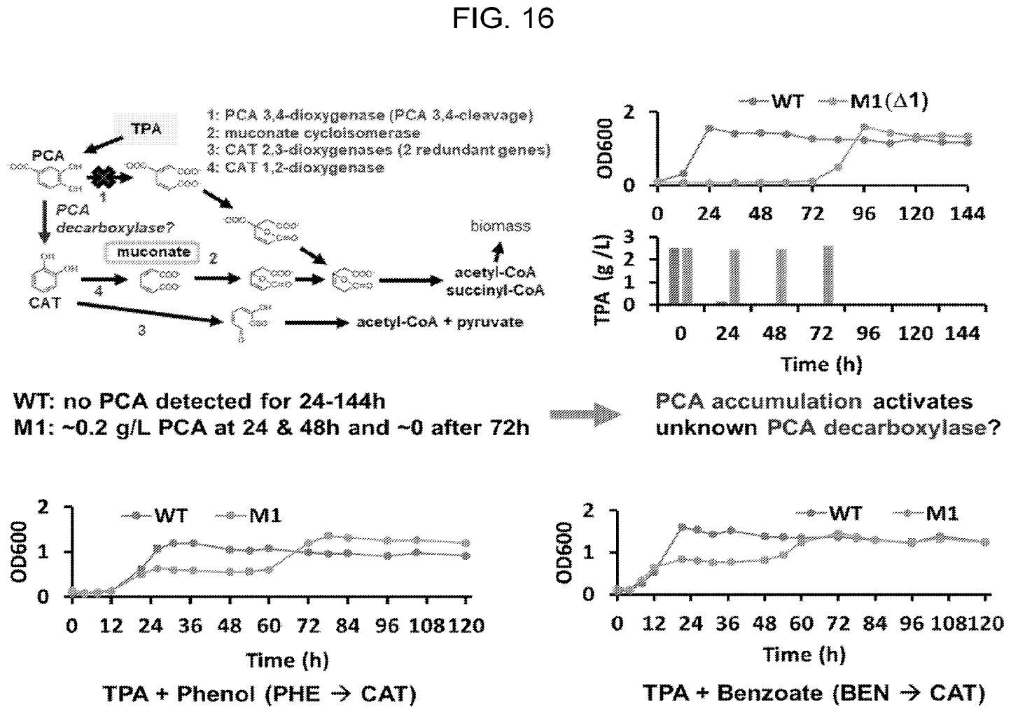

It was first asked why RPET can use high TPA concentration. To answer this, comparative genomics and functional analyses were performed. Unlike other TPA utilizing strains that are assumed to have one TPA catabolic pathway, our analysis suggests 3 redundant TPA pathways via 1, 4+2 or 3, explaining its robust growth at high TPA level (see e.g., FIG. 16).

For example, KO mutant M1 showed growth on TPA despite a long lag phase, implying the presence of unknown PCA decarboxylase.

Interestingly, we observed PCA accumulation up to 2 days in M1 and then consumption of PCA, implying PCA accumulation activates unknown PCA decarboxylase.

Furthermore, when we supplemented phenol or benzoate that is degraded via the CAT branch of beta-ketoadipate pathway, we found interesting bi-phasic growth, first using phenol or benzoate and then TPA later.

Based on our analysis, we built a metabolic map. To confirm this map's accuracy, we decided to grow the triple KO strain on multiple compounds as its sole carbon source. For example, as expected, no growth was observed when TPA or muconate was used, while we observed 100% muconate yield per benzoate when glucose was supplemented as the growth substrate. Given these results including the long lag phase of the M1 mutant, we hypothesized the native PCA decarboxylase is the bottleneck step and we are currently optimizing heterologous PCA decarboxylases for efficient TPA-to-MA production.

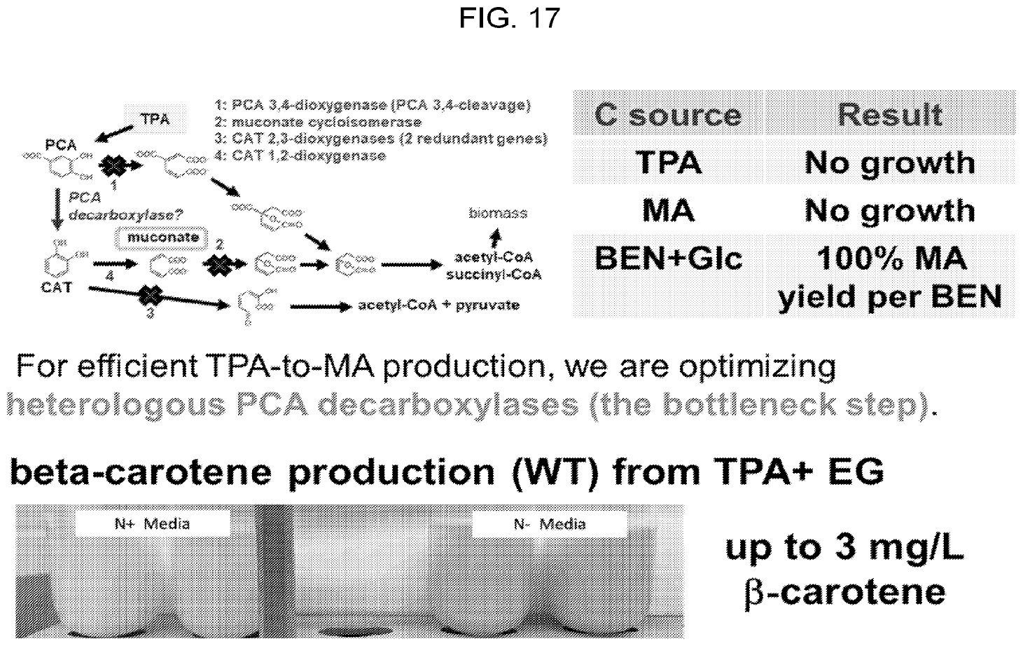

Interestingly, we observed significant beta-carotene production from the PET hydrolysis products when they were fed to wild-type RPET, especially under nitrogen-limiting conditions. This result is consistent with other Rhodococcus species data. To our knowledge, this is the first beta-carotene production from the PET hydrolysis products (see e.g., FIG. 17).

In summary, we discovered a novel salt-tolerant microbe that grew well up to 0.3 M TPA and 0.3 M EG. In addition, we built its metabolic map for PET utilization by comparative genomic and KO analysis and demonstrated that high-value carotene can be produced from the PET hydrolysis products.

A bigger metabolic map was built based on omics and preliminary metabolic modeling, which will help expand our product portfolio.

1-44. (canceled)

45. A system for waste polyethylene terephthalate (PET) valorization, the system comprising:
an engineered Rhodococcus sp. strain capable of utilizing PET hydrolysate as a carbon source; and
a medium comprising a PET hydrolysate, wherein the PET hydrolysate comprises at least one of terephthalic acid (TPA) and ethylene glycol (EG).

46. The system of claim 45, wherein the PET hydrolysate is selected from a PET alkaline hydrolysate, a PET chemical hydrolysate, and a PET enzymatic hydrolysate.

47. The system of claim 45, wherein the engineered Rhodococcus sp. strain produces a target product precursor selected from at least one of dimethylallyl diphosphate (DMAPP) and isopentenyl pyrophosphate (IPP).

48. The system of claim 45, wherein the engineered Rhodococcus sp. strain produces a target product selected from one or more of a carotenoid, lycopene, muconate, astaxanthin, and β-carotene.

49. The system of claim 45, wherein the medium comprises TPA and EG, each independently at a concentration of between about 5 mM and 400 mM.

50. The system of claim 45, wherein the PET hydrolysate is diluted to at least 5-fold.

51. The system of claim 45, wherein the engineered Rhodococcus sp. strain yields up to 37% biomass per used PET, as measured by dry cell weight (DCW) of biomass per consumed weight of PET.

52. A method for generating a target product from waste polyethylene terephthalate (PET) valorization, the method comprising:
providing an engineered Rhodococcus sp. strain capable of utilizing PET hydrolysate as a carbon source; and
incubating the engineered Rhodococcus sp. Strain in a medium comprising a PET hydrolysate, wherein the PET hydrolysate comprises at least one of terephthalic acid (TPA) and ethylene glycol (EG).

53. The method of claim 52, wherein the PET hydrolysate is selected from a PET alkaline hydrolysate, a PET chemical hydrolysate, and a PET enzymatic hydrolysate.

54. The method of claim 52, wherein the engineered Rhodococcus sp. strain produces a target product precursor selected from at least one of dimethylallyl diphosphate (DMAPP) and isopentenyl pyrophosphate (IPP).

55. The method of claim 52, wherein the engineered Rhodococcus sp. strain produces a target product selected from one or more of a carotenoid, lycopene, muconate, astaxanthin, and β-carotene.

56. The method of claim 52, wherein the medium comprises TPA and EG, each independently at a concentration of between about 5 mM and 400 mM.

57. The method of claim 52, wherein the PET hydrolysate is diluted to between about 5-fold and about 20-fold.

58. The method of claim 52, wherein the engineered Rhodococcus sp. strain yields up to 37% biomass per used PET, as measured by dry cell weight (DCW) of biomass per consumed weight of PET.

59. An engineered Rhodococcus sp. strain transformed with an artificial DNA construct, wherein the artificial DNA construct comprises:
(a) a promoter functional in the Rhodococcus sp. strain; and
(b) at least one polynucleotide selected from:
(i) a first polynucleotide comprising a nucleotide sequence encoding a first polypeptide having prenyltransferase—(CrtE), phytoene synthase—(CrtB), and phytoene desaturase—(CrtI) enzymatic activity);
(ii) a second polynucleotide comprising a nucleotide sequence encoding a second polypeptide having 1-deoxyxylulose-5-phosphate synthase enzymatic activity;
(iii) a third polynucleotide comprising a nucleotide sequence encoding a third polypeptide having isopentenyl pyrophosphate isomerase enzymatic activity; and
(iv) a fourth polynucleotide comprising a nucleotide sequence encoding a fourth polypeptide having 1-deoxyxylulose-5-phosphate synthase and isopentenyl pyrophosphate isomerase enzymatic activity; and
(c) a transcriptional termination sequence;
and wherein, a crtL-b gene in the engineered Rhodococcus sp. strain is knocked out or knocked down.

60. The engineered Rhodococcus sp. strain of claim 59, further comprising a heterologous PCA decarboxylase.

61. The engineered Rhodococcus sp. strain of claim 59, wherein the engineered Rhodococcus sp. strain produces at least one of a carotenoid, lycopene, muconate, astaxanthin, β-carotene, protocatechuic acid (PCA), gallic acid (GA), pyrogallol, catechol, muconic acid (MA), and vanillic acid (VA) from a PET hydrolysate carbon source.

62. The engineered Rhodococcus sp. strain of claim 61, wherein the PET hydrolysate carbon source comprises at least one of terephthalic acid (TPA) and ethylene glycol (EG).

63. The engineered Rhodococcus sp. strain of claim 61, wherein the PET hydrolysate carbon source is selected from a PET alkaline hydrolysate, a PET chemical hydrolysate, and a PET enzymatic hydrolysate.

64. The engineered Rhodococcus sp. strain of claim 59, further comprising at least one of:
a C. glutamicum-sourced dxs gene;
a C. glutamicum isopentenyl pyrophosphate isomerase gene (idi); and
an idi gene co-expressed with a dxs gene.


SEQUENCE LISTING (15 sequences)

SEQ ID NO: 1 - DNA, 4776 bp, artificial sequence
  pDD57 plasmid
actagttgattagctaagcagaaggccatcctgacggatggcctttttgcgtttaatactgtttaaactctagaaatattggatcgtcggcaccgtcacggccgtgggaggcggcacgatccgcgacgtgatgatcggccgcatccccacggtgctgcgcagtgagctctacgccatcccggcgttgatctgtgcgttcgctgtgcgggctctaacacgtcctagtatggtaggatgagcaacatttcgacgccgagagattcgccgcccgaaatgagcacgatccgcatgcttaattaagaaggagatatacatatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttgacctatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggctccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgagctggatccagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcgtctaccaggacttttacctgtccgacccgttgcaacggaaccccccacggaacccccgcgacacccgctccccaattgcgttagaacagcggtggattgtcggcttcgttgtgggccttttgagccgcttcctgttctgccgcacgctctttcctcgcccgatagccgagtcgcttaacggtgtccagatgcagcccgaaatgtttggccgtttgcggccaagagtggccctcgtcgtcgtgataggcgcggatgcgttcgcggcgtgcagcctgctcggcgagccactcgctgcgttcctgcgccacgagccggacgacgtggcgttcggatagtccggtgattcgagcgccttcggcggcggtcacgcgccgctttttgcggacagtcggctgccggttgtagccgtcgctgtagccgtcgctcatagcaatgcctccatggctgacgcggactttgcgcgccgcgcaactgtgctcgccgccgtgcgcgctgctgcgcccttccgcgagatggccgactggcgcgcactgagtgtggcctcgtagaccacgatcccgtccgcccaaatgcgcgacttggttgtgatccaacgccaaatgctgttggcgatggcgcggacctcgctgtccggtagcggtccgggacacacgtcgttgcacgggaactcggcgtttcgcgcgtggcactcggcatagatcgcgcggccgagtccgtccacgttccgggtcggcaggtagatccgcatgagggcgggacgataggcccacaacctgacggaatcgaacagtgcgcaattccgccctagcggcgtcggagccgctttgtacgtggtctgctgacgccagcgcggcggtggcatgttcgcgccgagctcggcctcgatgtggctgagtgtgtagagatctgagtggagccattccgtttcccaggcgatgtggccggggtttttggtcatgaggcctgagtaactgcggtcgccgtccacggcgcgccgaaggccttcggcgcacgccgccatgtatgcgagcggcttacgccgcgcgtattcggtgcgtggaacaggggcgttgagtgcccacactgcgtgtgcgtggccgttggcgcgattgcccacgatcgcgttgggcagcggatgggacccccgggcgctgagcgctcggagcgctgcgtctggatggtctacgtccacgaccagcaggtttgccagcgctgttgggttcgcctcgatgtaccggcggcctagggccgacgcgcggctttggcggtagatcccctcgagcagatcgtcgcttgccagcggccagtacggcagccagagctgctcaaattcgtcggcgacgtggctcacgcttggtagtagaccacgattaatcaccggtgtatggtccgacacgagctccaagtcagatatttcgctgaggggccaccccacaactgcacactcccccgctctcccgtcgagccctggtggtggaacaccagcgacagccgagcacccccaaccacctgtaccaacc

SEQ ID NO: 2 - DNA, 4913 bp, artificial sequence
  pGZ001 plasmid
actagttgattagctaagcagaaggccatcctgacggatggcctttttgcgtttaatactgtttaaactctagaaatattggatcgtcggcaccgtcacggccgtgggaggcggcacgatccgcgacgtgatgatcggccgcatccccacggtgctgcgcagtgagctctacgccatcccggcgttgatctgtgcgttcgctgagtggtgtgacttccgactcacgtgcggaggccgtgcgtacggagtgcgaaaccgtcatcagggcgcgaggtgggcgcttccgaatctgactccgctggtcgggcccgtacggccgggttgcgcgcgaatcgctgcgaatccgggcgaataccgttcggtccgcaccgttactcttggagtgttccgctgagagcgggagcacacccatcgggggaggtgccactgtaacgccaggatattcagcaacgatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttgacctatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggctccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgagctggatccagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcgtctaccaggacttttacctgtccgacccgttgcaacggaaccccccacggaacccccgcgacacccgctccccaattgcgttagaacagcggtggattgtcggcttcgttgtgggccttttgagccgcttcctgttctgccgcacgctctttcctcgcccgatagccgagtcgcttaacggtgtccagatgcagcccgaaatgtttggccgtttgcggccaagagtggccctcgtcgtcgtgataggcgcggatgcgttcgcggcgtgcagcctgctcggcgagccactcgctgcgttcctgcgccacgagccggacgacgtggcgttcggatagtccggtgattcgagcgccttcggcggcggtcacgcgccgctttttgcggacagtcggctgccggttgtagccgtcgctgtagccgtcgctcatagcaatgcctccatggctgacgcggactttgcgcgccgcgcaactgtgctcgccgccgtgcgcgctgctgcgcccttccgcgagatggccgactggcgcgcactgagtgtggcctcgtagaccacgatcccgtccgcccaaatgcgcgacttggttgtgatccaacgccaaatgctgttggcgatggcgcggacctcgctgtccggtagcggtccgggacacacgtcgttgcacgggaactcggcgtttcgcgcgtggcactcggcatagatcgcgcggccgagtccgtccacgttccgggtcggcaggtagatccgcatgagggcgggacgataggcccacaacctgacggaatcgaacagtgcgcaattccgccctagcggcgtcggagccgctttgtacgtggtctgctgacgccagcgcggcggtggcatgttcgcgccgagctcggcctcgatgtggctgagtgtgtagagatctgagtggagccattccgtttcccaggcgatgtggccggggtttttggtcatgaggcctgagtaactgcggtcgccgtccacggcgcgccgaaggccttcggcgcacgccgccatgtatgcgagcggcttacgccgcgcgtattcggtgcgtggaacaggggcgttgagtgcccacactgcgtgtgcgtggccgttggcgcgattgcccacgatcgcgttgggcagcggatgggacccccgggcgctgagcgctcggagcgctgcgtctggatggtctacgtccacgaccagcaggtttgccagcgctgttgggttcgcctcgatgtaccggcggcctagggccgacgcgcggctttggcggtagatcccctcgagcagatcgtcgcttgccagcggccagtacggcagccagagctgctcaaattcgtcggcgacgtggctcacgcttggtagtagaccacgattaatcaccggtgtatggtccgacacgagctccaagtcagatatttcgctgaggggccaccccacaactgcacactcccccgctctcccgtcgagccctggtggtggaacaccagcgacagccgagcacccccaaccacctgtaccaacc

SEQ ID NO: 3 - DNA, 5060 bp, artificial sequence
  pGZ002 plasmid
actagttgattagctaagcagaaggccatcctgacggatggcctttttgcgtttaatactgtttaaactctagaaatattggatcgtcggcaccgtcacggccgtgggaggcggcacgatccgcgacgtgatgatcggccgcatccccacggtgctgcgcagtgagctctacgccatcccggcgttgatctgtgcgttcgcttgatgtctccgggctgtcgtagggtacgggcgagtagcttccacaccgccacgacctgcgcatttgatgataacgagtccttttccggggctgtcgcccggggtgtcgtgacccccgcgccaatccgctgtttcgtgcctgttcgctacaagatcaactttttgtgtgaattgttcggatttcgactgttgcgttcgcggagttcatggttgactacaactggtcgcagcttctgtgttcgtaggcttgaaagagtgaacgttcgcaggatcgatgttgcgaacgcggtgctctgcttttcctgagggggaaatctagaagtacagcggtcggaaccggcccggcggactgtatgtgctgtccgccgaatccggtggttagaaagagaaggaaaagcatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttgacctatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggctccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgagctggatccagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcgtctaccaggacttttacctgtccgacccgttgcaacggaaccccccacggaacccccgcgacacccgctccccaattgcgttagaacagcggtggattgtcggcttcgttgtgggccttttgagccgcttcctgttctgccgcacgctctttcctcgcccgatagccgagtcgcttaacggtgtccagatgcagcccgaaatgtttggccgtttgcggccaagagtggccctcgtcgtcgtgataggcgcggatgcgttcgcggcgtgcagcctgctcggcgagccactcgctgcgttcctgcgccacgagccggacgacgtggcgttcggatagtccggtgattcgagcgccttcggcggcggtcacgcgccgctttttgcggacagtcggctgccggttgtagccgtcgctgtagccgtcgctcatagcaatgcctccatggctgacgcggactttgcgcgccgcgcaactgtgctcgccgccgtgcgcgctgctgcgcccttccgcgagatggccgactggcgcgcactgagtgtggcctcgtagaccacgatcccgtccgcccaaatgcgcgacttggttgtgatccaacgccaaatgctgttggcgatggcgcggacctcgctgtccggtagcggtccgggacacacgtcgttgcacgggaactcggcgtttcgcgcgtggcactcggcatagatcgcgcggccgagtccgtccacgttccgggtcggcaggtagatccgcatgagggcgggacgataggcccacaacctgacggaatcgaacagtgcgcaattccgccctagcggcgtcggagccgctttgtacgtggtctgctgacgccagcgcggcggtggcatgttcgcgccgagctcggcctcgatgtggctgagtgtgtagagatctgagtggagccattccgtttcccaggcgatgtggccggggtttttggtcatgaggcctgagtaactgcggtcgccgtccacggcgcgccgaaggccttcggcgcacgccgccatgtatgcgagcggcttacgccgcgcgtattcggtgcgtggaacaggggcgttgagtgcccacactgcgtgtgcgtggccgttggcgcgattgcccacgatcgcgttgggcagcggatgggacccccgggcgctgagcgctcggagcgctgcgtctggatggtctacgtccacgaccagcaggtttgccagcgctgttgggttcgcctcgatgtaccggcggcctagggccgacgcgcggctttggcggtagatcccctcgagcagatcgtcgcttgccagcggccagtacggcagccagagctgctcaaattcgtcggcgacgtggctcacgcttggtagtagaccacgattaatcaccggtgtatggtccgacacgagctccaagtcagatatttcgctgaggggccaccccacaactgcacactcccccgctctcccgtcgagccctggtggtggaacaccagcgacagccgagcacccccaaccacctgtaccaacc

SEQ ID NO: 4 - DNA, 4921 bp, artificial sequence
  pGZ003 plasmid
actagttgattagctaagcagaaggccatcctgacggatggcctttttgcgtttaatactgtttaaactctagaaatattggatcgtcggcaccgtcacggccgtgggaggcggcacgatccgcgacgtgatgatcggccgcatccccacggtgctgcgcagtgagctctacgccatcccggcgttgatctgtgcgttcgctcgcgtcagcttaaggttccgttcaccggtcggacgaattctcacgacatccagtgtcacctcgtcagcattttttcgggtgcccgaactttcctgtgagggaatcgctcggtgaccccaactgttgcgcagggtgtaccaatgagattgggtatatacccgagaacgattcgccaaaggcgggtgtcagacgcgtgcccacgtgtcgtcggcacccggaaacacaagggctcacacgcccatagatgaagggaccctcatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttgacctatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggctccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgagctggatccagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcgtctaccaggacttttacctgtccgacccgttgcaacggaaccccccacggaacccccgcgacacccgctccccaattgcgttagaacagcggtggattgtcggcttcgttgtgggccttttgagccgcttcctgttctgccgcacgctctttcctcgcccgatagccgagtcgcttaacggtgtccagatgcagcccgaaatgtttggccgtttgcggccaagagtggccctcgtcgtcgtgataggcgcggatgcgttcgcggcgtgcagcctgctcggcgagccactcgctgcgttcctgcgccacgagccggacgacgtggcgttcggatagtccggtgattcgagcgccttcggcggcggtcacgcgccgctttttgcggacagtcggctgccggttgtagccgtcgctgtagccgtcgctcatagcaatgcctccatggctgacgcggactttgcgcgccgcgcaactgtgctcgccgccgtgcgcgctgctgcgcccttccgcgagatggccgactggcgcgcactgagtgtggcctcgtagaccacgatcccgtccgcccaaatgcgcgacttggttgtgatccaacgccaaatgctgttggcgatggcgcggacctcgctgtccggtagcggtccgggacacacgtcgttgcacgggaactcggcgtttcgcgcgtggcactcggcatagatcgcgcggccgagtccgtccacgttccgggtcggcaggtagatccgcatgagggcgggacgataggcccacaacctgacggaatcgaacagtgcgcaattccgccctagcggcgtcggagccgctttgtacgtggtctgctgacgccagcgcggcggtggcatgttcgcgccgagctcggcctcgatgtggctgagtgtgtagagatctgagtggagccattccgtttcccaggcgatgtggccggggtttttggtcatgaggcctgagtaactgcggtcgccgtccacggcgcgccgaaggccttcggcgcacgccgccatgtatgcgagcggcttacgccgcgcgtattcggtgcgtggaacaggggcgttgagtgcccacactgcgtgtgcgtggccgttggcgcgattgcccacgatcgcgttgggcagcggatgggacccccgggcgctgagcgctcggagcgctgcgtctggatggtctacgtccacgaccagcaggtttgccagcgctgttgggttcgcctcgatgtaccggcggcctagggccgacgcgcggctttggcggtagatcccctcgagcagatcgtcgcttgccagcggccagtacggcagccagagctgctcaaattcgtcggcgacgtggctcacgcttggtagtagaccacgattaatcaccggtgtatggtccgacacgagctccaagtcagatatttcgctgaggggccaccccacaactgcacactcccccgctctcccgtcgagccctggtggtggaacaccagcgacagccgagcacccccaaccacctgtaccaacc

SEQ ID NO: 5 - DNA, 4963 bp, artificial sequence
  pGZ004 plasmid
actagttgattagctaagcagaaggccatcctgacggatggcctttttgcgtttaatactgtttaaactctagaaatattggatcgtcggcaccgtcacggccgtgggaggcggcacgatccgcgacgtgatgatcggccgcatccccacggtgctgcgcagtgagctctacgccatcccggcgttgatctgtgcgttcgctgctgcgatcctatcgacctgccgtagcgtggcgtgccgacgcgcgggtccgcgtcggcggcctcgctacgggaaggcctcgaaacaaggcgccgaaaaaagatgtgggcatccgggaacaaatctccgcacccctccgttgagccttacgacaacatgagcgtgcaagactcaagttcgaattgactcccgacggtgtcggagtgcaaacttgagcggagggcgctcactaagcgccaacatcgcagttcagtaatgaaaaacctgcccacaggactgcaaaacgaaagtgaggaacactatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttgacctatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggctccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgagctggatccagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcgtctaccaggacttttacctgtccgacccgttgcaacggaaccccccacggaacccccgcgacacccgctccccaattgcgttagaacagcggtggattgtcggcttcgttgtgggccttttgagccgcttcctgttctgccgcacgctctttcctcgcccgatagccgagtcgcttaacggtgtccagatgcagcccgaaatgtttggccgtttgcggccaagagtggccctcgtcgtcgtgataggcgcggatgcgttcgcggcgtgcagcctgctcggcgagccactcgctgcgttcctgcgccacgagccggacgacgtggcgttcggatagtccggtgattcgagcgccttcggcggcggtcacgcgccgctttttgcggacagtcggctgccggttgtagccgtcgctgtagccgtcgctcatagcaatgcctccatggctgacgcggactttgcgcgccgcgcaactgtgctcgccgccgtgcgcgctgctgcgcccttccgcgagatggccgactggcgcgcactgagtgtggcctcgtagaccacgatcccgtccgcccaaatgcgcgacttggttgtgatccaacgccaaatgctgttggcgatggcgcggacctcgctgtccggtagcggtccgggacacacgtcgttgcacgggaactcggcgtttcgcgcgtggcactcggcatagatcgcgcggccgagtccgtccacgttccgggtcggcaggtagatccgcatgagggcgggacgataggcccacaacctgacggaatcgaacagtgcgcaattccgccctagcggcgtcggagccgctttgtacgtggtctgctgacgccagcgcggcggtggcatgttcgcgccgagctcggcctcgatgtggctgagtgtgtagagatctgagtggagccattccgtttcccaggcgatgtggccggggtttttggtcatgaggcctgagtaactgcggtcgccgtccacggcgcgccgaaggccttcggcgcacgccgccatgtatgcgagcggcttacgccgcgcgtattcggtgcgtggaacaggggcgttgagtgcccacactgcgtgtgcgtggccgttggcgcgattgcccacgatcgcgttgggcagcggatgggacccccgggcgctgagcgctcggagcgctgcgtctggatggtctacgtccacgaccagcaggtttgccagcgctgttgggttcgcctcgatgtaccggcggcctagggccgacgcgcggctttggcggtagatcccctcgagcagatcgtcgcttgccagcggccagtacggcagccagagctgctcaaattcgtcggcgacgtggctcacgcttggtagtagaccacgattaatcaccggtgtatggtccgacacgagctccaagtcagatatttcgctgaggggccaccccacaactgcacactcccccgctctcccgtcgagccctggtggtggaacaccagcgacagccgagcacccccaaccacctgtaccaacc

SEQ ID NO: 6 - DNA, 5123 bp, artificial sequence
  pGZ005 plasmid
actagttgattagctaagcagaaggccatcctgacggatggcctttttgcgtttaatactgtttaaactctagaaatattggatcgtcggcaccgtcacggccgtgggaggcggcacgatccgcgacgtgatgatcggccgcatccccacggtgctgcgcagtgagctctacgccatcccggcgttgatctgtgcgttcgctgaggaaaagctagctcatatatgttacgtctcgtaacagataatgggcgcagaggcagtctgatgaaccacacccccgcatgccgagtgacgccggttactatgacggtcggggtccggccgcgatttggcggttgtgcaggccatgccgtaatctgtcctgcggagttcaatgcagtagttgaacttcaccccaaatcatgttctacccaagaccgttggtgatcactcccgcaagggtgtgattgaaagtccgggaatgtccggcggcccacgcaggaggacgaggtagggggatgcgtatcggttgagggctcgcgccctgaccgtatcgtttcctttcacgccccgtgtgccctgcaccggggcgttttgcatgtcgtggattgtgtgctccgggattccggcggtgcgcagcggttccgaagtgggataacgactgtcatgagaggaggcgaagtatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttgacctatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggctccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgagctggatccagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcgtctaccaggacttttacctgtccgacccgttgcaacggaaccccccacggaacccccgcgacacccgctccccaattgcgttagaacagcggtggattgtcggcttcgttgtgggccttttgagccgcttcctgttctgccgcacgctctttcctcgcccgatagccgagtcgcttaacggtgtccagatgcagcccgaaatgtttggccgtttgcggccaagagtggccctcgtcgtcgtgataggcgcggatgcgttcgcggcgtgcagcctgctcggcgagccactcgctgcgttcctgcgccacgagccggacgacgtggcgttcggatagtccggtgattcgagcgccttcggcggcggtcacgcgccgctttttgcggacagtcggctgccggttgtagccgtcgctgtagccgtcgctcatagcaatgcctccatggctgacgcggactttgcgcgccgcgcaactgtgctcgccgccgtgcgcgctgctgcgcccttccgcgagatggccgactggcgcgcactgagtgtggcctcgtagaccacgatcccgtccgcccaaatgcgcgacttggttgtgatccaacgccaaatgctgttggcgatggcgcggacctcgctgtccggtagcggtccgggacacacgtcgttgcacgggaactcggcgtttcgcgcgtggcactcggcatagatcgcgcggccgagtccgtccacgttccgggtcggcaggtagatccgcatgagggcgggacgataggcccacaacctgacggaatcgaacagtgcgcaattccgccctagcggcgtcggagccgctttgtacgtggtctgctgacgccagcgcggcggtggcatgttcgcgccgagctcggcctcgatgtggctgagtgtgtagagatctgagtggagccattccgtttcccaggcgatgtggccggggtttttggtcatgaggcctgagtaactgcggtcgccgtccacggcgcgccgaaggccttcggcgcacgccgccatgtatgcgagcggcttacgccgcgcgtattcggtgcgtggaacaggggcgttgagtgcccacactgcgtgtgcgtggccgttggcgcgattgcccacgatcgcgttgggcagcggatgggacccccgggcgctgagcgctcggagcgctgcgtctggatggtctacgtccacgaccagcaggtttgccagcgctgttgggttcgcctcgatgtaccggcggcctagggccgacgcgcggctttggcggtagatcccctcgagcagatcgtcgcttgccagcggccagtacggcagccagagctgctcaaattcgtcggcgacgtggctcacgcttggtagtagaccacgattaatcaccggtgtatggtccgacacgagctccaagtcagatatttcgctgaggggccaccccacaactgcacactcccccgctctcccgtcgagccctggtggtggaacaccagcgacagccgagcacccccaaccacctgtaccaacc

SEQ ID NO: 7 - DNA, 4913 bp, artificial sequence
  pGZ006 plasmid
actagttgattagctaagcagaaggccatcctgacggatggcctttttgcgtttaatactgtttaaactctagaaatattggatcgtcggcaccgtcacggccgtgggaggcggcacgatccgcgacgtgatgatcggccgcatccccacggtgctgcgcagtgagctctacgccatcccggcgttgatctgtgcgttcgctcaggtctgcgcgatgcgctgatcgcccgggcgcaccggttccgtcccgaccttgcactcaccctgtccgagtgctaaaaatgcacttggcactcacgacgcgtgagtgccaggtcgggacggtgagaccgggaaccaaagacacccctggtcgtccgtcgcgggcaccgaactcggccgaaggcgtaaatgaggcgacccgactagcggtcgccttgtgtgtcacccccaatccggaggatcacttcgcaatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttgacctatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggctccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgagctggatccagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcgtctaccaggacttttacctgtccgacccgttgcaacggaaccccccacggaacccccgcgacacccgctccccaattgcgttagaacagcggtggattgtcggcttcgttgtgggccttttgagccgcttcctgttctgccgcacgctctttcctcgcccgatagccgagtcgcttaacggtgtccagatgcagcccgaaatgtttggccgtttgcggccaagagtggccctcgtcgtcgtgataggcgcggatgcgttcgcggcgtgcagcctgctcggcgagccactcgctgcgttcctgcgccacgagccggacgacgtggcgttcggatagtccggtgattcgagcgccttcggcggcggtcacgcgccgctttttgcggacagtcggctgccggttgtagccgtcgctgtagccgtcgctcatagcaatgcctccatggctgacgcggactttgcgcgccgcgcaactgtgctcgccgccgtgcgcgctgctgcgcccttccgcgagatggccgactggcgcgcactgagtgtggcctcgtagaccacgatcccgtccgcccaaatgcgcgacttggttgtgatccaacgccaaatgctgttggcgatggcgcggacctcgctgtccggtagcggtccgggacacacgtcgttgcacgggaactcggcgtttcgcgcgtggcactcggcatagatcgcgcggccgagtccgtccacgttccgggtcggcaggtagatccgcatgagggcgggacgataggcccacaacctgacggaatcgaacagtgcgcaattccgccctagcggcgtcggagccgctttgtacgtggtctgctgacgccagcgcggcggtggcatgttcgcgccgagctcggcctcgatgtggctgagtgtgtagagatctgagtggagccattccgtttcccaggcgatgtggccggggtttttggtcatgaggcctgagtaactgcggtcgccgtccacggcgcgccgaaggccttcggcgcacgccgccatgtatgcgagcggcttacgccgcgcgtattcggtgcgtggaacaggggcgttgagtgcccacactgcgtgtgcgtggccgttggcgcgattgcccacgatcgcgttgggcagcggatgggacccccgggcgctgagcgctcggagcgctgcgtctggatggtctacgtccacgaccagcaggtttgccagcgctgttgggttcgcctcgatgtaccggcggcctagggccgacgcgcggctttggcggtagatcccctcgagcagatcgtcgcttgccagcggccagtacggcagccagagctgctcaaattcgtcggcgacgtggctcacgcttggtagtagaccacgattaatcaccggtgtatggtccgacacgagctccaagtcagatatttcgctgaggggccaccccacaactgcacactcccccgctctcccgtcgagccctggtggtggaacaccagcgacagccgagcacccccaaccacctgtaccaacc

SEQ ID NO: 8 - DNA, 4926 bp, artificial sequence
  pGZ007 plasmid
actagttgattagctaagcagaaggccatcctgacggatggcctttttgcgtttaatactgtttaaactctagaaatattggatcgtcggcaccgtcacggccgtgggaggcggcacgatccgcgacgtgatgatcggccgcatccccacggtgctgcgcagtgagctctacgccatcccggcgttgatctgtgcgttcgctgcacaccatccttcgcatcgtggccgtgggtatcggaagtcgaattctgccgggcgggcggctccgtcggggaccgcatttggcctggtgagcaatttcccgtaacatggatcaacggtgcgctatgcgtgccggttgtctgcgtgcccagtgccggattcgatccggattgttcatgtttcggccggtgaagatcccattgggcgccacggtaacggaaacagatgcaataagcacgccaaaccacatggatcgcggaggatatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttgacctatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggctccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgagctggatccagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcgtctaccaggacttttacctgtccgacccgttgcaacggaaccccccacggaacccccgcgacacccgctccccaattgcgttagaacagcggtggattgtcggcttcgttgtgggccttttgagccgcttcctgttctgccgcacgctctttcctcgcccgatagccgagtcgcttaacggtgtccagatgcagcccgaaatgtttggccgtttgcggccaagagtggccctcgtcgtcgtgataggcgcggatgcgttcgcggcgtgcagcctgctcggcgagccactcgctgcgttcctgcgccacgagccggacgacgtggcgttcggatagtccggtgattcgagcgccttcggcggcggtcacgcgccgctttttgcggacagtcggctgccggttgtagccgtcgctgtagccgtcgctcatagcaatgcctccatggctgacgcggactttgcgcgccgcgcaactgtgctcgccgccgtgcgcgctgctgcgcccttccgcgagatggccgactggcgcgcactgagtgtggcctcgtagaccacgatcccgtccgcccaaatgcgcgacttggttgtgatccaacgccaaatgctgttggcgatggcgcggacctcgctgtccggtagcggtccgggacacacgtcgttgcacgggaactcggcgtttcgcgcgtggcactcggcatagatcgcgcggccgagtccgtccacgttccgggtcggcaggtagatccgcatgagggcgggacgataggcccacaacctgacggaatcgaacagtgcgcaattccgccctagcggcgtcggagccgctttgtacgtggtctgctgacgccagcgcggcggtggcatgttcgcgccgagctcggcctcgatgtggctgagtgtgtagagatctgagtggagccattccgtttcccaggcgatgtggccggggtttttggtcatgaggcctgagtaactgcggtcgccgtccacggcgcgccgaaggccttcggcgcacgccgccatgtatgcgagcggcttacgccgcgcgtattcggtgcgtggaacaggggcgttgagtgcccacactgcgtgtgcgtggccgttggcgcgattgcccacgatcgcgttgggcagcggatgggacccccgggcgctgagcgctcggagcgctgcgtctggatggtctacgtccacgaccagcaggtttgccagcgctgttgggttcgcctcgatgtaccggcggcctagggccgacgcgcggctttggcggtagatcccctcgagcagatcgtcgcttgccagcggccagtacggcagccagagctgctcaaattcgtcggcgacgtggctcacgcttggtagtagaccacgattaatcaccggtgtatggtccgacacgagctccaagtcagatatttcgctgaggggccaccccacaactgcacactcccccgctctcccgtcgagccctggtggtggaacaccagcgacagccgagcacccccaaccacctgtaccaacc

SEQ ID NO: 9 - DNA, 4913 bp, artificial sequence
  pGZ008 plasmid
actagttgattagctaagcagaaggccatcctgacggatggcctttttgcgtttaatactgtttaaactctagaaatattggatcgtcggcaccgtcacggccgtgggaggcggcacgatccgcgacgtgatgatcggccgcatccccacggtgctgcgcagtgagctctacgccatcccggcgttgatctgtgcgttcgctagcccgtactcctcgtacaggcgcagcgctgcgatcccgaacccggacagctcggcggccaccgagatgctgtaaacgccacgtaccgagggcaagtcggcatcacgcccggattctgcggccatgtcgctccgatcgggaattttctcgcgcaggcccttgtcattgcctactgaagatgctataccaaatctacagcatctgctggagtttacctgatggaatgagatggtttcaggaggtggacacgatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttgacctatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggctccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgagctggatccagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcgtctaccaggacttttacctgtccgacccgttgcaacggaaccccccacggaacccccgcgacacccgctccccaattgcgttagaacagcggtggattgtcggcttcgttgtgggccttttgagccgcttcctgttctgccgcacgctctttcctcgcccgatagccgagtcgcttaacggtgtccagatgcagcccgaaatgtttggccgtttgcggccaagagtggccctcgtcgtcgtgataggcgcggatgcgttcgcggcgtgcagcctgctcggcgagccactcgctgcgttcctgcgccacgagccggacgacgtggcgttcggatagtccggtgattcgagcgccttcggcggcggtcacgcgccgctttttgcggacagtcggctgccggttgtagccgtcgctgtagccgtcgctcatagcaatgcctccatggctgacgcggactttgcgcgccgcgcaactgtgctcgccgccgtgcgcgctgctgcgcccttccgcgagatggccgactggcgcgcactgagtgtggcctcgtagaccacgatcccgtccgcccaaatgcgcgacttggttgtgatccaacgccaaatgctgttggcgatggcgcggacctcgctgtccggtagcggtccgggacacacgtcgttgcacgggaactcggcgtttcgcgcgtggcactcggcatagatcgcgcggccgagtccgtccacgttccgggtcggcaggtagatccgcatgagggcgggacgataggcccacaacctgacggaatcgaacagtgcgcaattccgccctagcggcgtcggagccgctttgtacgtggtctgctgacgccagcgcggcggtggcatgttcgcgccgagctcggcctcgatgtggctgagtgtgtagagatctgagtggagccattccgtttcccaggcgatgtggccggggtttttggtcatgaggcctgagtaactgcggtcgccgtccacggcgcgccgaaggccttcggcgcacgccgccatgtatgcgagcggcttacgccgcgcgtattcggtgcgtggaacaggggcgttgagtgcccacactgcgtgtgcgtggccgttggcgcgattgcccacgatcgcgttgggcagcggatgggacccccgggcgctgagcgctcggagcgctgcgtctggatggtctacgtccacgaccagcaggtttgccagcgctgttgggttcgcctcgatgtaccggcggcctagggccgacgcgcggctttggcggtagatcccctcgagcagatcgtcgcttgccagcggccagtacggcagccagagctgctcaaattcgtcggcgacgtggctcacgcttggtagtagaccacgattaatcaccggtgtatggtccgacacgagctccaagtcagatatttcgctgaggggccaccccacaactgcacactcccccgctctcccgtcgagccctggtggtggaacaccagcgacagccgagcacccccaaccacctgtaccaacc

SEQ ID NO: 10 - DNA, 4913 bp, artificial sequence
  pGZ009 plasmid
actagttgattagctaagcagaaggccatcctgacggatggcctttttgcgtttaatactgtttaaactctagaaatattggatcgtcggcaccgtcacggccgtgggaggcggcacgatccgcgacgtgatgatcggccgcatccccacggtgctgcgcagtgagctctacgccatcccggcgttgatctgtgcgttcgctgcgtcaactggtccaggagggcgttccaggcctgccggcgatgcaccaccccgtgcacgagtacgagggtggggccggagccggcgatgtcgtgaggcaagtccatggacagatgttaccgctatcacgagtaacatctactgccagccgatagtggggcccgtcgcccacggatgtgggcgacgggccccgacatcgaccccgattcgggtcggtcgggccgatgcgggaaagatggtaaccggtctgtatggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttgacctatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggctccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgagctggatccagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcgtctaccaggacttttacctgtccgacccgttgcaacggaaccccccacggaacccccgcgacacccgctccccaattgcgttagaacagcggtggattgtcggcttcgttgtgggccttttgagccgcttcctgttctgccgcacgctctttcctcgcccgatagccgagtcgcttaacggtgtccagatgcagcccgaaatgtttggccgtttgcggccaagagtggccctcgtcgtcgtgataggcgcggatgcgttcgcggcgtgcagcctgctcggcgagccactcgctgcgttcctgcgccacgagccggacgacgtggcgttcggatagtccggtgattcgagcgccttcggcggcggtcacgcgccgctttttgcggacagtcggctgccggttgtagccgtcgctgtagccgtcgctcatagcaatgcctccatggctgacgcggactttgcgcgccgcgcaactgtgctcgccgccgtgcgcgctgctgcgcccttccgcgagatggccgactggcgcgcactgagtgtggcctcgtagaccacgatcccgtccgcccaaatgcgcgacttggttgtgatccaacgccaaatgctgttggcgatggcgcggacctcgctgtccggtagcggtccgggacacacgtcgttgcacgggaactcggcgtttcgcgcgtggcactcggcatagatcgcgcggccgagtccgtccacgttccgggtcggcaggtagatccgcatgagggcgggacgataggcccacaacctgacggaatcgaacagtgcgcaattccgccctagcggcgtcggagccgctttgtacgtggtctgctgacgccagcgcggcggtggcatgttcgcgccgagctcggcctcgatgtggctgagtgtgtagagatctgagtggagccattccgtttcccaggcgatgtggccggggtttttggtcatgaggcctgagtaactgcggtcgccgtccacggcgcgccgaaggccttcggcgcacgccgccatgtatgcgagcggcttacgccgcgcgtattcggtgcgtggaacaggggcgttgagtgcccacactgcgtgtgcgtggccgttggcgcgattgcccacgatcgcgttgggcagcggatgggacccccgggcgctgagcgctcggagcgctgcgtctggatggtctacgtccacgaccagcaggtttgccagcgctgttgggttcgcctcgatgtaccggcggcctagggccgacgcgcggctttggcggtagatcccctcgagcagatcgtcgcttgccagcggccagtacggcagccagagctgctcaaattcgtcggcgacgtggctcacgcttggtagtagaccacgattaatcaccggtgtatggtccgacacgagctccaagtcagatatttcgctgaggggccaccccacaactgcacactcccccgctctcccgtcgagccctggtggtggaacaccagcgacagccgagcacccccaaccacctgtaccaacc

SEQ ID NO: 11 - DNA, 4026 bp, artificial sequence
  pYH038 plasmid
gtctgaccacccaccacgggtaacgcggaggggctcggcgagatgcatctcgccgagcccctccgcgtcggttcaggcgcgcttgcgttccgaactggatgatttcgctgtggccttcttcgccgccgtcttggccggagctttcttcgccggcttcttcttggccggtgcggccttcttcccggcggcgtcgacgctgcgctgcagggccgccaccagatccaccacttcggcgtcctcgccgctcttctcgacctcggcgacggggatgaccttcttgccgcccgactcgatcatctcgtcgagcagttcacgcaactgcacctggtagtcgtcggtgaattcggtggggtcgaagtcgtcggccatgctgtcgacgagcgtctcggccatcgcgagttccttcgccttcggcttgtcgacgccgtcgagcgaggagaactcggcgacgcgtacctcgtccggccacagcagcgtctggatcaccagcaccccgtcgcgtgaccgcatcgcggccagtcgggtgcgctgccggagggtgaagtggacgagggctgtgcggtcgctgttctcgagggcagtcgcgagaaggacatacgccttcggagagctggaatccggttcgaggaagtaactcttctcgaacagaatgggatcgatctgttcggtcggcacgaactgcagaacgggaatctcgtgtttctcggcggcaggcagtttcccgaaatcatcgtccgtcaacaccacgcgcacgccgtcgtcggagtcgtaggccttgtcgatatcggcgtattggacgacgttgccgcattcggagcagacgcggttgtatttgatccggccgccgtccttggcatggacctggtgaaacctgatgtcgtgatcctcggttgccgaatagaccttgaccggcacattcacgagtccgaacgctatcgagcccttccatatcgaccgcatgcgttccatgatggtctccttttcgcgttcgcgcgacagtgaatagaggtccgctgtgacacaagaatccctgttacttctcgaccgtattgattcggatgattcctacgcgagcctgcggaacgaccaggaattctgggagccgctggcccgccgagccctggaggagctcgggctgccggtgccgccggtgctgcgggtgcccggcgagagcaccaaccccgtactggtcggcgagcccgacccggtgatcaagctgttcggcgagcactggtgcggtccggagagcctcgcgtcggagtcggaggcgtacgcggtcctggcggacgccccggtgccggtgccccgcctcctcggccgcggcgagctgcggcccggcaccggagcctggccgtggccctacctggtgatgagccggatgaccggcaccacctggcggtccgcgatggacggcacgaccgaccggaacgcgctgctcgccctggcccgcgaactcggccgggtgctcggccggctgcacagggtgccgctgaccgggaacaccgtgctcaccccccattccgaggtcttcccggaactgctgcgggaacgccgcgcggcgaccgtcgaggaccaccgcgggtggggctacctctcgccccggctgctggaccgcctggaggactggctgccggacgtggacacgctgctggccggccgcgaaccccggttcgtccacggcgacctgcacgggaccaacatcttcgtggacctggccgcgaccgaggtcaccgggatcgtcgacttcaccgacgtctatgcgggagactcccgctacagcctggtgcaactgcatctcaacgccttccggggcgaccgcgagatcctggccgcgctgctcgacggggcgcagtggaagcggaccgaggacttcgcccgcgaactgctcgccttcaccttcctgcacgacttcgaggtgttcgaggagaccccgctggatctctccggcttcaccgatccggaggaactggcgcagttcctctgggggccgccggacaccgcccccggcgcctgagtcgcgctcccgtcgactcgccacggccggcccggaccagtgggggcgttgaggggagtgctgacgcgtggcccggctaagcgggcgtgggggagcgcggtgcgttggacgagatgcaattgcgcggggcggggaacccggatgcgcctgcgaggtcccgaaacatgccggtcgcgcttcccggtccaccaggttgggagtcggtgtactgagtcgggcccggacggaacatcctagtttcccgaactaccgtggtggcattcactcatcccaccaaccgtcgtcaccgcagcgcacacctcgcgactgcatcacacccgatcgccaggccgacgacgaagaggccagaggctttctacgagcggatacggccgcgaacgttagcgggatcactggcgggcgtgttccgagtcgccggcggtcgggtaaaattgctggttcgtgtgttctttgcgcagtttttctcattcaacgcgagcgtcattttcctaggagtcttgttgatcaccgctaccgacctggaagttcgagctggagtgcggacgttgctgtccgcgcccgggccggctttgcgggtgcagcccggcgaccgcatcgggctggtcggccgcaacggtgccggcaagacgaccaccctccgcattctcgcgggggagggggagccgtatgcgggtgccgtggtgcgcaccggcgacctcggatacctcccgcaggacccgaaagaaggcgacctcgacgtcctcgcgaaggaccgcgtgttgtccgcgcgcggcctcgacgcgctgctgcgggacatggagaagcagcagatcctgatgtcggaggtcgtcgacgacgccgagcgcgacaaggcggtgcgcaagtacggcaatctcgaggaccgcttctcggccctcggggggtacgaggcagagagcgaagccgcacgcatctgcaacagcctcggcctggccgaccgcatcctcggccagcagctgcacaccctgtcgggtggtcagcgtcgccgacatcaccgatggggaagatcgggctcgccacttcgggctcatgatcctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagccatgggcaaatattatacgcaaggcgacaaggtgctgatgccgctggcgattcaggttcatcatgtaggtgtaaaacgagttagcccttagtgactcgaattctctagtcga

SEQ ID NO: 12 - DNA, 8659 bp, artificial sequence
  pJD078 plasmid
ggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcgtctaccaggacttttacctgtccgacccgttgcaacggaaccccccacggaacccccgcgacacccgctccccaattgcgttagaacagcggtggattgtcggcttcgttgtgggccttttgagccgcttcctgttctgccgcacgctctttcctcgcccgatagccgagtcgcttaacggtgtccagatgcagcccgaaatgtttggccgtttgcggccaagagtggccctcgtcgtcgtgataggcgcggatgcgttcgcggcgtgcagcctgctcggcgagccactcgctgcgttcctgcgccacgagccggacgacgtggcgttcggatagtccggtgattcgagcgccttcggcggcggtcacgcgccgctttttgcggacagtcggctgccggttgtagccgtcgctgtagccgtcgctcatagcaatgcctccatggctgacgcggactttgcgcgccgcgcaactgtgctcgccgccgtgcgcgctgctgcgcccttccgcgagatggccgactggcgcgcactgagtgtggcctcgtagaccacgatcccgtccgcccaaatgcgcgacttggttgtgatccaacgccaaatgctgttggcgatggcgcggacctcgctgtccggtagcggtccgggacacacgtcgttgcacgggaactcggcgtttcgcgcgtggcactcggcatagatcgcgcggccgagtccgtccacgttccgggtcggcaggtagatccgcatgagggcgggacgataggcccacaacctgacggaatcgaacagtgcgcaattccgccctagcggcgtcggagccgctttgtacgtggtctgctgacgccagcgcggcggtggcatgttcgcgccgagctcggcctcgatgtggctgagtgtgtagagatctgagtggagccattccgtttcccaggcgatgtggccggggtttttggtcatgaggcctgagtaactgcggtcgccgtccacggcgcgccgaaggccttcggcgcacgccgccatgtatgcgagcggcttacgccgcgcgtattcggtgcgtggaacaggggcgttgagtgcccacactgcgtgtgcgtggccgttggcgcgattgcccacgatcgcgttgggcagcggatgggacccccgggcgctgagcgctcggagcgctgcgtctggatggtctacgtccacgaccagcaggtttgccagcgctgttgggttcgcctcgatgtaccggcggcctagggccgacgcgcggctttggcggtagatcccctcgagcagatcgtcgcttgccagcggccagtacggcagccagagctgctcaaattcgtcggcgacgtggctcacgcttggtagtagaccacgattaatcaccggtgtatggtccgacacgagctccaagtcagatatttcgctgaggggccaccccacaactgcacactcccccgctctcccgtcgagccctggtggtggaacaccagcgacagccgagcacccccaaccacctgtaccaaccactagttgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacagcggataaagtagcaaagagaaggaggttaggaatgacggtctgcgcaaaaaaacacgttcatctcactcgcgatgctgcggagcagttactggctgatattgatcgacgccttgatcagttattgcccgtggagggagaacgggatgttgtgggtgccgcgatgcgtgaaggtgcgctggcaccgggaaaacgtattcgccccatgttgctgttgctgaccgcccgcgatctgggttgcgctgtcagccatgacggattactggatttggcctgtgcggtggaaatggtccacgcggcttcgctgatccttgacgatatgccctgcatggacgatgcgaagctgcggcgcggacgccctaccattcattctcattacggagagcatgtggcaatactggcggcggttgccttgctgagtaaagcctttggcgtaattgccgatgcagatggcctcacgccgctggcaaaaaatcgggcggtttctgaactgtcaaacgccatcggcatgcaaggattggttcagggtcagttcaaggatctgtctgaaggggataagccgcgcagcgctgaagctattttgatgacgaatcactttaaaaccagcacgctgttttgtgcctccatgcagatggcctcgattgttgcgaatgcctccagcgaagcgcgtgattgcctgcatcgtttttcacttgatcttggtcaggcatttcaactgctggacgatttgaccgatggcatgaccgacaccggtaaggatagcaatcaggacgccggtaaatcgacgctggtcaatctgttaggcccgagggcggttgaagaacgtctgagacaacatcttcagcttgccagtgagcatctctctgcggcctgccaacacgggcacgccactcaacattttattcaggcctggtttgacaaaaaactcgctgccgtcagttaataatactagagctcaaggaggtactagatgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctaataatactagagctcaaggaggtactagatgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaagcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcaaactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatataaggtctggtgtcaaaaataatactagagccaggcatcaaataaaacgaaaggctcagtcgaaagactgggcctttcgttttatctgttgtttgtcggtgaacgctctctactagagccaggcatcaaataaaacgaaaggctcagtcgaaagactgggcctttcgttttatctgttgtttgtcggtgaacgctctctactagtagcggccgctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcagg

SEQ ID NO: 13 - DNA, 6488 bp, artificial sequence
  pJD095 plasmid
gatagtgagttatgcaaccaaatgacgtggctttgttgaataaatcgaacttttgctgagttgaaggatcagctcgatataaacgcagaaaggcccacccgaaggtgagccagtgtgactctagtagagagcgttcaccgacaaacaacagataaaacgaaaggcccagtctttcgactgagcctttcgttttatttgatgcctggttatcatgagaccggtcttattccccgaacagggaatccagccatccaacaacagtggtttcaatgccgtcggcgtcgaggccataatcggcgagcacttcattgcgggacgcgtgatccaggtacttctggggcacggcgatttgtcggcgaggggtatccacctcagaggcgttaagcgcatcagagagcaaggatcccacgccgccgtggatgacgccgtcttcgatggtgatcacgaggtcatgatcatcagacagcgcgaccaaggactgcgggatggggacaatccagcgggggtcaacaaccgtgacgttcacgccgtgctgtttaatcctggaagcaacgtcaagtgcaacagttgcgcgctcgcctaccgcaatgatgagaactgatggcgcatcgtcggttgattcaacgtcagtggcgtcttcatatgcgaggacatccacgccgtcttccaaggtgtcgatagcaacaattggagttggcaagtcgcccttggggaaacgcacaactgtggggccatcatcgatggaaatagcctcattgagcagctcacgcaaggaatcctcatcacgtggtgccgccacctgcacgcctggaacgatcgaggtcagcgccatatcccagacgccattgtggctcgctccatccgaacccgtgacacctgagcgatcaagcaccaaagtaacaggctggttgagcatgcccacatccatgagcagctgatcaaaagcgcggttcaagaacgtggagtaaatagccaccacagggtgttttccacccaatgcgaggcctgcggcagaagttaccgcgtgctgctcagcaatgccgacatcaaagaatcggttggggaaattggcttcgaacttggacagaccggtaggacctgccatcgcggcggtgatggcaacaacgttttcattctgcgcaccaatcttgaccagctcatcgctgaacacagaggtccaaccgggctttgatgcagatttaggagctcctgtgagcggatcgatgacgcccgtggagtgcatcaattcgtccaaatcctgctcagcaggcgcgtaaccacgacccttttcggtgaccatgtgcacgatgatggggccatcataatcatgagcgtatttcagcgcattgtcgacagctttttggttatgtccatcaaccggacccacgtatttcatgcccagttcagggaacatttcggtgggaatgacggtgctcttcacaccttctttaaatgcatggagcgcttcaaaagtacgctcccctacccaccccatggatttcagggacgtcttgcccttttccatgaagcgatcatagaaaggctgcatgcgaaggcccgcaaggttttccgcaaatccgccaatggttggagaataactccggccattgtcattgactacgacaacaactttgcggtctttaccagcagcaatattgttcagtgcttcccaacacatgccgccagttagagcgccatcaccaacgacagcaaccacactatgcgtggtatcgccatccaactgcttggctttagacaaaccatccgcataagacaaggccgccgaagcatgcgaagactcagtccaatcgtgctcactttcagcacggcaggtgtaaccagaaaggccatctttttgacgcaaagaatcaaaatctttagcgcgacccgtcaggatcttatgcacataggactggtgagaagtatcaaagatgatcgggtcttgaggcgaatcgaaaactcgatgaagaccgatggttaattccactacgcccaaatttggacctaagtggccaccagttgctgcgactttatcgaccaggaaagttcggatttctttggcaagagcgtccaaatcctcatcattaagggcctttaagtcagcaggtgttgaaatactgttcagaattcccatgcttttccttctctttctaaccaccggattcggcggacagcacatacagtccgccgggccggttccgaccgctgtacttctagatttccccctcaggaaaagcagagcaccgcgttcgcaacatcgatcctgcgaacgttcactctttcaagcctacgaacacagaagctgcgaccagttgtagtcaaccatgaactccgcgaacgcaacagtcgaaatccgaacaattcacacaaaaagttgatcttgtagcgaacaggcacgaaacagcggattggcgcgggggtcacgacaccccgggcgacagccccggaaaaggactcgttatcatcaaatgcgcaggtcgtggcggtgtggaagctactcgcccgtaccctacgacagcccggagacatcactcgaggtgaagacgaaagggcctcgtgatacgcctatttttataggttaatgtcatgataataatggtttcttagacgtcgatatctggcgaaaatggttaatcaagggcactctcgactgtcttctttatcatgcaactcgtaggacaggtgccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtgggccgcaaagcgctccggcttaactcaggcgttagtcccatcacccgctttatgcgcgcagccacttatttcagcgctccgccctgttccaaaggctttctagccttttgggcctccagcgcttgtgcagcaagcgccaccagctatccttcttgcagcgctgcgccgctttcgtggtccagcggtttttcttgggctgcgcccttcttcaaggccgggcgctcccttttggccttcgggtctaactctgcggtcaagcggacccgcattctgcgggccgcttaccttggccgttagacatcatggtaaatctgcgcagacagccctgtgcagctgaaacgcggttacgtatagcttgccatatgtctagccatacgtaaccgcaggtaaaaggcatatttttcgcgtgtcatggctagtaaataacaccggtgtcatttagagtcagggaaagacaatgaaaaacgaagaaagccaccgggcggcaacccgatgactttcgcttatcacccagcacacacctgggagaaatcacggtcatgagtttacagactcatgcgcagaatgcgcacactaaaacacctacccgcgtcgagcgcgaccgtggtggactggacaacaccccagcatctgccagtgaccgcgaccttttacgcgatcatctaggccgcgatgtactccacggttcagtcacacgagactttaaaaaggcctatcgacgcaacgctgacggcacgaactcgccgcgtatgtatcgcttcgagactgatgctttaggacggtgcgagtacgccatgctcaccaccaagcagtacgccgccgtcctggtcgtagacgttgaccaagtaggtaccgcaggcggtgaccccgcagacttaaacccgtacgtccgcgacgtggtgcgctcactgattactcatagcgtcgggccagcctgggtgggtattaacccaactaacggcaaagcccagttcatatggcttattgaccctgtctacgctgaccgtaacggtaaatctgcgcagatgaagcttcttgcagcaaccacgcgtgtgctgggtgagcttttagaccatgacccgcacttttcccaccgctttagccgcaacccgttctacacaggcaaagcccctaccgcttatcgttggtataggcagcacaaccgggtgatgcgccttggagacttgataaagcaggtaagggatatggcaggacacgaccagttcaaccccaccccacgccagcaattcagctctggccgcgaacttatcaacgcggtcaagacccgccgtgaagaagcccaagcattcaaagcactcgcccaggacgtagacgcggaaatcgccggtggtctcgaccagtatgacccggaacttatcgacggtgtgcgtgtgctctggattgtccaaggaaccgcagcacgcgacgaaacagcctttagacatgcgcttaagactggccaccgcttgcgccagcaaggccaacgcctgacagacgcagcaatcatcgacgcctatgagcacgcctacaacgtcgcacacacccacggcggtgcaggccgcgacaacgagatgccacccatgcgcgaccgccaaaccatggcaaggcgcgtgcgcgggtatgtcgcccaatccaagagcgagacctacagcggctctaacgcaccaggtaaagccaccagcagcgagcggaaagccttggccacgatgggacgcagaggcggacaaaaagccgcacaacgctggaaaacagaccccgagggcaaatatgcgcaagcacaaaggtcgaagcttgaaaagacgcaccgtaagaaaaaggctcaaggacgatctacgaagtcccgtattagccaaatggtgaacgatcagtatttccagacagggacagttcccacgtgggctgaaataggggcagaggtaggagtctctcgcgccacggttgctaggcatgtcgcggagctaaagaagagcggtgactatccggacgtttaaggggtctcataccgtaagcaatatacggttcccctgccgttaggcagttagataaaacctcacttgaagaaaaccttgaggggcagggcagcttatatgcttcaaagcatgacttcctctgttctcctagacctcgcaaccctccgccataacctcaccgaattgcttaagtttcacccctttgacggcttaaccgccgcataggaaatgccctccggactaattgtctggagggcatttttgctacctgctgtgcactttgtacataaggcaacaagattttggcaaaaggtggaagggcgaattgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacg

SEQ ID NO: 14 - DNA, 5165 bp, artificial sequence
  pJD096 plasmid
gatagtgagttatgcaaccaaatgacgtggctttgttgaataaatcgaacttttgctgagttgaaggatcagctcgatataaacgcagaaaggcccacccgaaggtgagccagtgtgactctagtagagagcgttcaccgacaaacaacagataaaacgaaaggcccagtctttcgactgagcctttcgttttatttgatgcctggttatcatgagaccggtcttactctgcgtcaaacgcttccaggatggcttggcgcagctcaggggcgctaagctgatccactagccatggagaaaacacaaatggtgtggcatcaacagcgtcgaaaagcttctgcggttccgcccactcgaactcctccacctcatcatccagtggctccacaaattcccccaccgctaaacgcacgaggtggaccgggcacaactcccactccacaatgccggacgcgtcgacagcacggtactggtaatcaggcagaatctcttgaatatccaagaaagaatctacctccagccccaactcatcgacacccctgcgacgaatcgcatccgcgtttgtctcatccggaccagggtgcccacacatagagttcgtccacacaccaggccatgtcttcttcgacaatgcacgacgcgtcaccaacagctccccacgcgggttcaaaatataggtggaaaacgcgaaatgcagaggcgtgtccttagtgtgcaccgtagctttcggcgcagtaccaataggattgccctcggaatcagctaaaacaaccagttcaacctcagtagtcatgcccctaagcttagacatgcttttccttctctttctaaccaccggattcggcggacagcacatacagtccgccgggccggttccgaccgctgtacttctagatttccccctcaggaaaagcagagcaccgcgttcgcaacatcgatcctgcgaacgttcactctttcaagcctacgaacacagaagctgcgaccagttgtagtcaaccatgaactccgcgaacgcaacagtcgaaatccgaacaattcacacaaaaagttgatcttgtagcgaacaggcacgaaacagcggattggcgcgggggtcacgacaccccgggcgacagccccggaaaaggactcgttatcatcaaatgcgcaggtcgtggcggtgtggaagctactcgcccgtaccctacgacagcccggagacatcactcgaggtgaagacgaaagggcctcgtgatacgcctatttttataggttaatgtcatgataataatggtttcttagacgtcgatatctggcgaaaatggttaatcaagggcactctcgactgtcttctttatcatgcaactcgtaggacaggtgccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtgggccgcaaagcgctccggcttaactcaggcgttagtcccatcacccgctttatgcgcgcagccacttatttcagcgctccgccctgttccaaaggctttctagccttttgggcctccagcgcttgtgcagcaagcgccaccagctatccttcttgcagcgctgcgccgctttcgtggtccagcggtttttcttgggctgcgcccttcttcaaggccgggcgctcccttttggccttcgggtctaactctgcggtcaagcggacccgcattctgcgggccgcttaccttggccgttagacatcatggtaaatctgcgcagacagccctgtgcagctgaaacgcggttacgtatagcttgccatatgtctagccatacgtaaccgcaggtaaaaggcatatttttcgcgtgtcatggctagtaaataacaccggtgtcatttagagtcagggaaagacaatgaaaaacgaagaaagccaccgggcggcaacccgatgactttcgcttatcacccagcacacacctgggagaaatcacggtcatgagtttacagactcatgcgcagaatgcgcacactaaaacacctacccgcgtcgagcgcgaccgtggtggactggacaacaccccagcatctgccagtgaccgcgaccttttacgcgatcatctaggccgcgatgtactccacggttcagtcacacgagactttaaaaaggcctatcgacgcaacgctgacggcacgaactcgccgcgtatgtatcgcttcgagactgatgctttaggacggtgcgagtacgccatgctcaccaccaagcagtacgccgccgtcctggtcgtagacgttgaccaagtaggtaccgcaggcggtgaccccgcagacttaaacccgtacgtccgcgacgtggtgcgctcactgattactcatagcgtcgggccagcctgggtgggtattaacccaactaacggcaaagcccagttcatatggcttattgaccctgtctacgctgaccgtaacggtaaatctgcgcagatgaagcttcttgcagcaaccacgcgtgtgctgggtgagcttttagaccatgacccgcacttttcccaccgctttagccgcaacccgttctacacaggcaaagcccctaccgcttatcgttggtataggcagcacaaccgggtgatgcgccttggagacttgataaagcaggtaagggatatggcaggacacgaccagttcaaccccaccccacgccagcaattcagctctggccgcgaacttatcaacgcggtcaagacccgccgtgaagaagcccaagcattcaaagcactcgcccaggacgtagacgcggaaatcgccggtggtctcgaccagtatgacccggaacttatcgacggtgtgcgtgtgctctggattgtccaaggaaccgcagcacgcgacgaaacagcctttagacatgcgcttaagactggccaccgcttgcgccagcaaggccaacgcctgacagacgcagcaatcatcgacgcctatgagcacgcctacaacgtcgcacacacccacggcggtgcaggccgcgacaacgagatgccacccatgcgcgaccgccaaaccatggcaaggcgcgtgcgcgggtatgtcgcccaatccaagagcgagacctacagcggctctaacgcaccaggtaaagccaccagcagcgagcggaaagccttggccacgatgggacgcagaggcggacaaaaagccgcacaacgctggaaaacagaccccgagggcaaatatgcgcaagcacaaaggtcgaagcttgaaaagacgcaccgtaagaaaaaggctcaaggacgatctacgaagtcccgtattagccaaatggtgaacgatcagtatttccagacagggacagttcccacgtgggctgaaataggggcagaggtaggagtctctcgcgccacggttgctaggcatgtcgcggagctaaagaagagcggtgactatccggacgtttaaggggtctcataccgtaagcaatatacggttcccctgccgttaggcagttagataaaacctcacttgaagaaaaccttgaggggcagggcagcttatatgcttcaaagcatgacttcctctgttctcctagacctcgcaaccctccgccataacctcaccgaattgcttaagtttcacccctttgacggcttaaccgccgcataggaaatgccctccggactaattgtctggagggcatttttgctacctgctgtgcactttgtacataaggcaacaagattttggcaaaaggtggaagggcgaattgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacg

SEQ ID NO: 15 - DNA, 7105 bp, artificial sequence
  pJD097 plasmid
gatagtgagttatgcaaccaaatgacgtggctttgttgaataaatcgaacttttgctgagttgaaggatcagctcgatataaacgcagaaaggcccacccgaaggtgagccagtgtgactctagtagagagcgttcaccgacaaacaacagataaaacgaaaggcccagtctttcgactgagcctttcgttttatttgatgcctggttatcatgagaccggtcttactctgcgtcaaacgcttccaggatggcttggcgcagctcaggggcgctaagctgatccactagccatggagaaaacacaaatggtgtggcatcaacagcgtcgaaaagcttctgcggttccgcccactcgaactcctccacctcatcatccagtggctccacaaattcccccaccgctaaacgcacgaggtggaccgggcacaactcccactccacaatgccggacgcgtcgacagcacggtactggtaatcaggcagaatctcttgaatatccaagaaagaatctacctccagccccaactcatcgacacccctgcgacgaatcgcatccgcgtttgtctcatccggaccagggtgcccacacatagagttcgtccacacaccaggccatgtcttcttcgacaatgcacgacgcgtcaccaacagctccccacgcgggttcaaaatataggtggaaaacgcgaaatgcagaggcgtgtccttagtgtgcaccgtagctttcggcgcagtaccaataggattgccctcggaatcagctaaaacaaccagttcaacctcagtagtcatgcccctaagcttagacatatacgtccctcccttctcaaatttagttcttattccccgaacagggaatccagccatccaacaacagtggtttcaatgccgtcggcgtcgaggccataatcggcgagcacttcattgcgggacgcgtgatccaggtacttctggggcacggcgatttgtcggcgaggggtatccacctcagaggcgttaagcgcatcagagagcaaggatcccacgccgccgtggatgacgccgtcttcgatggtgatcacgaggtcatgatcatcagacagcgcgaccaaggactgcgggatggggacaatccagcgggggtcaacaaccgtgacgttcacgccgtgctgtttaatcctggaagcaacgtcaagtgcaacagttgcgcgctcgcctaccgcaatgatgagaactgatggcgcatcgtcggttgattcaacgtcagtggcgtcttcatatgcgaggacatccacgccgtcttccaaggtgtcgatagcaacaattggagttggcaagtcgcccttggggaaacgcacaactgtggggccatcatcgatggaaatagcctcattgagcagctcacgcaaggaatcctcatcacgtggtgccgccacctgcacgcctggaacgatcgaggtcagcgccatatcccagacgccattgtggctcgctccatccgaacccgtgacacctgagcgatcaagcaccaaagtaacaggctggttgagcatgcccacatccatgagcagctgatcaaaagcgcggttcaagaacgtggagtaaatagccaccacagggtgttttccacccaatgcgaggcctgcggcagaagttaccgcgtgctgctcagcaatgccgacatcaaagaatcggttggggaaattggcttcgaacttggacagaccggtaggacctgccatcgcggcggtgatggcaacaacgttttcattctgcgcaccaatcttgaccagctcatcgctgaacacagaggtccaaccgggctttgatgcagatttaggagctcctgtgagcggatcgatgacgcccgtggagtgcatcaattcgtccaaatcctgctcagcaggcgcgtaaccacgacccttttcggtgaccatgtgcacgatgatggggccatcataatcatgagcgtatttcagcgcattgtcgacagctttttggttatgtccatcaaccggacccacgtatttcatgcccagttcagggaacatttcggtgggaatgacggtgctcttcacaccttctttaaatgcatggagcgcttcaaaagtacgctcccctacccaccccatggatttcagggacgtcttgcccttttccatgaagcgatcatagaaaggctgcatgcgaaggcccgcaaggttttccgcaaatccgccaatggttggagaataactccggccattgtcattgactacgacaacaactttgcggtctttaccagcagcaatattgttcagtgcttcccaacacatgccgccagttagagcgccatcaccaacgacagcaaccacactatgcgtggtatcgccatccaactgcttggctttagacaaaccatccgcataagacaaggccgccgaagcatgcgaagactcagtccaatcgtgctcactttcagcacggcaggtgtaaccagaaaggccatctttttgacgcaaagaatcaaaatctttagcgcgacccgtcaggatcttatgcacataggactggtgagaagtatcaaagatgatcgggtcttgaggcgaatcgaaaactcgatgaagaccgatggttaattccactacgcccaaatttggacctaagtggccaccagttgctgcgactttatcgaccaggaaagttcggatttctttggcaagagcgtccaaatcctcatcattaagggcctttaagtcagcaggtgttgaaatactgttcagaattcccatgcttttccttctctttctaaccaccggattcggcggacagcacatacagtccgccgggccggttccgaccgctgtacttctagatttccccctcaggaaaagcagagcaccgcgttcgcaacatcgatcctgcgaacgttcactctttcaagcctacgaacacagaagctgcgaccagttgtagtcaaccatgaactccgcgaacgcaacagtcgaaatccgaacaattcacacaaaaagttgatcttgtagcgaacaggcacgaaacagcggattggcgcgggggtcacgacaccccgggcgacagccccggaaaaggactcgttatcatcaaatgcgcaggtcgtggcggtgtggaagctactcgcccgtaccctacgacagcccggagacatcactcgaggtgaagacgaaagggcctcgtgatacgcctatttttataggttaatgtcatgataataatggtttcttagacgtcgatatctggcgaaaatggttaatcaagggcactctcgactgtcttctttatcatgcaactcgtaggacaggtgccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtgggccgcaaagcgctccggcttaactcaggcgttagtcccatcacccgctttatgcgcgcagccacttatttcagcgctccgccctgttccaaaggctttctagccttttgggcctccagcgcttgtgcagcaagcgccaccagctatccttcttgcagcgctgcgccgctttcgtggtccagcggtttttcttgggctgcgcccttcttcaaggccgggcgctcccttttggccttcgggtctaactctgcggtcaagcggacccgcattctgcgggccgcttaccttggccgttagacatcatggtaaatctgcgcagacagccctgtgcagctgaaacgcggttacgtatagcttgccatatgtctagccatacgtaaccgcaggtaaaaggcatatttttcgcgtgtcatggctagtaaataacaccggtgtcatttagagtcagggaaagacaatgaaaaacgaagaaagccaccgggcggcaacccgatgactttcgcttatcacccagcacacacctgggagaaatcacggtcatgagtttacagactcatgcgcagaatgcgcacactaaaacacctacccgcgtcgagcgcgaccgtggtggactggacaacaccccagcatctgccagtgaccgcgaccttttacgcgatcatctaggccgcgatgtactccacggttcagtcacacgagactttaaaaaggcctatcgacgcaacgctgacggcacgaactcgccgcgtatgtatcgcttcgagactgatgctttaggacggtgcgagtacgccatgctcaccaccaagcagtacgccgccgtcctggtcgtagacgttgaccaagtaggtaccgcaggcggtgaccccgcagacttaaacccgtacgtccgcgacgtggtgcgctcactgattactcatagcgtcgggccagcctgggtgggtattaacccaactaacggcaaagcccagttcatatggcttattgaccctgtctacgctgaccgtaacggtaaatctgcgcagatgaagcttcttgcagcaaccacgcgtgtgctgggtgagcttttagaccatgacccgcacttttcccaccgctttagccgcaacccgttctacacaggcaaagcccctaccgcttatcgttggtataggcagcacaaccgggtgatgcgccttggagacttgataaagcaggtaagggatatggcaggacacgaccagttcaaccccaccccacgccagcaattcagctctggccgcgaacttatcaacgcggtcaagacccgccgtgaagaagcccaagcattcaaagcactcgcccaggacgtagacgcggaaatcgccggtggtctcgaccagtatgacccggaacttatcgacggtgtgcgtgtgctctggattgtccaaggaaccgcagcacgcgacgaaacagcctttagacatgcgcttaagactggccaccgcttgcgccagcaaggccaacgcctgacagacgcagcaatcatcgacgcctatgagcacgcctacaacgtcgcacacacccacggcggtgcaggccgcgacaacgagatgccacccatgcgcgaccgccaaaccatggcaaggcgcgtgcgcgggtatgtcgcccaatccaagagcgagacctacagcggctctaacgcaccaggtaaagccaccagcagcgagcggaaagccttggccacgatgggacgcagaggcggacaaaaagccgcacaacgctggaaaacagaccccgagggcaaatatgcgcaagcacaaaggtcgaagcttgaaaagacgcaccgtaagaaaaaggctcaaggacgatctacgaagtcccgtattagccaaatggtgaacgatcagtatttccagacagggacagttcccacgtgggctgaaataggggcagaggtaggagtctctcgcgccacggttgctaggcatgtcgcggagctaaagaagagcggtgactatccggacgtttaaggggtctcataccgtaagcaatatacggttcccctgccgttaggcagttagataaaacctcacttgaagaaaaccttgaggggcagggcagcttatatgcttcaaagcatgacttcctctgttctcctagacctcgcaaccctccgccataacctcaccgaattgcttaagtttcacccctttgacggcttaaccgccgcataggaaatgccctccggactaattgtctggagggcatttttgctacctgctgtgcactttgtacataaggcaacaagattttggcaaaaggtggaagggcgaattgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacg